SEQUENCE LISTING (44 sequences)

SEQ ID NO: 1 - DNA, 28 bp, Artificial Sequence
  Made in the lab
cgcgaattcaatgctgattttcggaagg

SEQ ID NO: 2 - DNA, 24 bp, Artificial Sequence
  Made in the lab
cgcctgcagtcaggttgggtgctg

SEQ ID NO: 3 - DNA, 20 bp, Artificial Sequence
  Made in the lab
ggcttacccatacgatgttc

SEQ ID NO: 4 - DNA, 1449 bp, Homo sapiens
gtcccactgtcctggctccgtgcgtccagtgagattgggcctgggcgctggagctgctgtggctcccgccgcggcgctgctcatggaggccatgcagagcccagaactcacgccgggggaggccgagacagccggcggtactcatagatgaggcagcggcggcgcggggcggcggcagccgggctctccatgagcaggcggcggcggcgacgggctgcggcggcacggcagttttcggtccccagggaggatgaagacactgtttgaagagatcaaagcatcaattaaaaataactataaccaagatcgatcattttgtaggcctgttcttccttgggggggtgtttttactatcaaagctggccgcaaagcagtatcctgtacaccactctatgttgaaataagactgaaaaatacctgcaccatagatggattcttgatgttattatatgtcattcttaatgaaaatgaaaacttccctagggaactctctcttcattttggtagagagtttgtagactgttttctttacttaatggacacctacagttttacaactgtgaagctactttggatttgggacaagatggaaaaacagcaatacaaatctgaagtccataaagcttcattaataattgatttgtttgggaatgagcatgataattttacaaaaaatcttgaaaatctcatgtctaccattcaagagagttactgttccaactggcgatgcccaactcgagtgcaggaggatcagcagcgcacaattaatataaatcctccccaagaaattccacatggaaacttgataagactggctgtgaatgagttattctgttccaagattgaactgtgtgaagagcatgggtgtggtggcttaagagaattttcccaacgaattttctgccatggggcacccccttttgttgtcttaaatatgcaacattggaaatctgaagatctggcgtatgtaccctattacttggatttgtctgatcacaagtatttgttggaaggtgccacattatttaacaaagaggaacatcattattctgcagctttccagattggtggacattggatgcactatgatgggctcagaaatgtgaatttaattttgttaaataaacccccagagtttctcctcttgtcatcattggtttatattcgagcaacagagaaataaatatagattgatgctaaaagttgttttccctcctgcccatgctctcccagatgaagggcttttattttgtgtatacttggtatccaagaaaatagttcaactatactagtttcagaagtgtattttcagtgtttaaccccaggtaaatgttttatatagaggatctgtgcaaaaatgtttgtaatttttttatatttcctgagttatttttatatgagcatattttatgttggaataaaatatatcttgtgaaaaaaaaaaaaaaa

SEQ ID NO: 5 - protein, 310 aa, Homo sapiens
MKTLFEEIKASIKNNYNQDRSFCRPVLPWGGVFTIKAGRKAVSCTPLYVEIRLKNTCTIDGFLMLLYVILNENENFPRELSLHFGREFVDCFLYLMDTYSFTTVKLLWIWDKMEKQQYKSEVHKASLIIDLFGNEHDNFTKNLENLMSTIQESYCSNWRCPTRVQEDQQRTININPPQEIPHGNLIRLAVNELFCSKIELCEEHGCGGLREFSQRIFCHGAPPFVVLNMQHWKSEDLAYVPYYLDLSDHKYLLEGATLFNKEEHHYSAAFQIGGHWMHYDGLRNVNLILLNKPPEFLLLSSLVYIRATEK

SEQ ID NO: 6 - DNA, 2801 bp, Homo sapiens
agcaactccaatgaaatagacatcgtgcatcatgtagacactgaggccaatatagctacggaggtttgcctcactattctggacctgttatccctcttcacacagactcatcagagacaactccaacaatgtgactgtcaaaattcattgatgaaaagggtctttgatacctacatgctctttttccaagtcaatcagtcagccacagcgctgaagcatgtgtttgcctccttgagactgtttgtatgcaagtttccttcagcgttctttcaagggcctgctgacctctgtggatcattctgttacgaagtcctaaaatgctgtaaccacaggtcacggtcaactcagacagaagcctcagcccttctgtactttttcatgaggaagaattttgaatttaacaagcagaagtcaattgtccggtcccacttacaactcatcaaagctgtgagccagttaatagccgatgctgggattggaggctctcggtttcaacattcgcttgcaattaccaataatttcgccaatggagataagcaaatgaaaaacagcaatttcccagcagaggtgaaggacctgactaagcgtataaggactgttttgatggccacagctcagatgaaggagcacgagaaggaccccgagatgctggtggatctccagtacagcctggcaaactcctacgcaagcactcctgaactacgcaggacctggctggaaagtatggccaagattcatgccagaaacggagatttatctgaggctgccatgtgttacatccatattgctgctctcattgcagagtatctgaaaagaaagggcatgttctctatgggatggccagcttttttgagcattacaccaaacattaaggaagaaggagcgatgaaagaggattctggaatgcaagatacaccatacaatgagaatatcctggtggagcagctatacatgtgtgtggagtttctctggaagtctgagcgatatgaactcattgctgatgtcaacaagcccatcattgctgtctttgagaaacaacgagacttcaaaaaattgtcagatctctactacgacattcatcggtcatatctgaaagtggcagaggtggtgaattcggagaagcggctgtttggtcgctactatcgtgtggcattttatgggcaggctgtgggcttttttgaagaagaagaaggtaaagagtatatttataaagagcctaagctgacaggtctgtccgagatttcccaaagattactcaagctctatgcagataaatttggagcagacaatgtgaagataatccaggattccaacaaggtaaaccccaaggatttggaccccaaatatgcctacatccaggtgacctatgtgacgccgttctttgaggaaaaggaaatcgaagaccggaagacagatttcgaaatgcaccacaacatcaaccgctttgtcttcgagacacccttcacgctgtcgggcaagaagcacggtggggtggcggagcagtgcaagcggcggacgatcctgacaacgagtcacctgttcccctacgtgaagaagagaatacaagtaattagccaatcgagcacagaactgaatccaattgaagtggcaattgacgagatgtccaagaaggtttctgagcttaatcagctttgcacaatggaagaagtggacatgatcagactgcagctcaaactgcaaggaagtgtcagcgtgaaggttaatgctgggccaatggcctatgcacgagcttttcttgaagaaaccaatgcaaagaagtaccctgacaaccaagtaaagcttttgaaggagatcttcaggcaatttgcagatgcatgtgggcaggcccttgacgtgaatgagcgcctcatcaaagaggaccagctggagtaccaggaagaactgaggtcccactacaaggacatgctcagcgaactctccacagtcatgaatgagcagctctgtcgaggtccgtgtttatacagcttctgttcctctgtgtctagtatttccctcagtactgtaagcaaaagtgattacgggcagggacgacctgtcaaagcgcggagtggaccaaacctgcactcgagtaattagcaaagcaactccggccctacccacggtctccatctcatctagtgctgaagtctgagggctctgcagcatcagacccacctctaagagaactttctgaatttgcagctaatctcggggaagagaaagataggtttaatttatttgaagttttcatggtgttaatatttttgtttacctcgctagcttcagaattttgccaacctctgaatttgcacattttgtataatttttttttctttgagcagtgttgatcaagccaagttgaatatttgccatgaaattccagtgaatgtgtagctcaaatgcaaaccctaagtttgctgtcagttattgtatggtcagtaccccagtcctagtacacatattttaaaggttaaagtgaatgtttttgtaacatttaagcatatttcagatgtaaataaaagattgtaaaatatacggtttttaccaaatttaaaagatcctttttagttaatactatgacagtactaaaaatatatgaataacatttcagataccattatattaaaatatttgtgtatgtgtacaaaagcgttgataaatactaatctttaaagtttgtggagttcctttatttgtaatatatgtgctcttaaaagcaatgggatgtgaaattatgaaagtattttattgttcatagaaataaaaaacacagttactttgcaaaaaaaaaaaaaaaaaaa

SEQ ID NO: 7 - protein, 698 aa, Homo sapiens
SNSNEIDIVHHVDTEANIATEVCLTILDLLSLFTQTHQRQLQQCDCQNSLMKRVFDTYMLFFQVNQSATALKHVFASLRLFVCKFPSAFFQGPADLCGSFCYEVLKCCNHRSRSTQTEASALLYFFMRKNFEFNKQKSIVRSHLQLIKAVSQLIADAGIGGSRFQHSLAITNNFANGDKQMKNSNFPAEVKDLTKRIRTVLMATAQMKEHEKDPEMLVDLQYSLANSYASTPELRRTWLESMAKIHARNGDLSEAAMCYIHIAALIAEYLKRKGMFSMGWPAFLSITPNIKEEGAMKEDSGMQDTPYNENILVEQLYMCVEFLWKSERYELIADVNKPIIAVFEKQRDFKKLSDLYYDIHRSYLKVAEVVNSEKRLFGRYYRVAFYGQAVGFFEEEEGKEYIYKEPKLTGLSEISQRLLKLYADKFGADNVKIIQDSNKVNPKDLDPKYAYIQVTYVTPFFEEKEIEDRKTDFEMHHNINRFVFETPFTLSGKKHGGVAEQCKRRTILTTSHLFPYVKKRIQVISQSSTELNPIEVAIDEMSKKVSELNQLCTMEEVDMIRLQLKLQGSVSVKVNAGPMAYARAFLEETNAKKYPDNQVKLLKEIFRQFADACGQALDVNERLIKEDQLEYQEELRSHYKDMLSELSTVMNEQLCRGPCLYSFCSSVSSISLSTVSKSDYGQGRPVKARSGPNLHSSN

SEQ ID NO: 8 - DNA, 7290 bp, Homo sapiens
atggccggtgagcggacccgcaggttcacccggagcctgttgagacctgggcaggcggccgagctccggcacagcgccgcgtccgccgccgcggtggcagtcagcagccggcagcagcagcgggtgagtgcggcgcgtcctccgcgccttcccgcgcgccccaggcgcccggggggaacggagcccagcttccccaaaagctcggcgggcccaggtccccgcagctcacgggtcctctcctccgcactccctcccccgccgcctctgcgggctcccccggcgccacctcagcgaggcagcgccaccgacgctaaacagaccagtgcgctgagagagagggatgcagtccgtggtcctcgccctgtgctgggtgatttccgcgggaagtccctggggcagcagccagcatctgatgctaggatcctatccataccaacccagatgttcctgcgttgcagtaggtccacactgccggggagaggtggcaagagacaaatcaactctagtggaaacgggaagcgagtgggaacaacagcctcacagcaagaccggggctcagcagccgcgctcttggcctgcgtttggtggaccggaatccggaggagctgtccacacatttgtagacgacgccaagaggaaccatcccacgaaattttgcctctggaaagtgacgatgaaatgaatagacccagaaccctcatcataatgcaagaaaagcctaggcttctcgagcctttggattatgagactgtcattgaagaacttgaaaagacctaccggaatgatcctcttcaagatctcttgttcttccccagtgatgacttttcagcagccacagtttcctgggatatccgcacgttgtactcaacagtacctgaagatgcagagcacaaggcagaaaatttactggttaaggaggcttgtaaattttatagttcccagtggcatgtggtaaactacaaatatgaacaatattctggagacattcgacagctaccccgagcagaatacaaaccagagaagcttccttcacattcctttgagattgaccatgaagatgctgataaggatgaagataccacttcccactcgtcttccaaggggggtggaggagcgggaggaactggtgttttcaagtccggctggctctacaaggggaattttaacagcaccgtgaacaacaccgttactgttcggtcattcaaaaagcgctacttccagctgactcagttaccagataactcctacattatgaacttttacaaagatgagaaaatatccaaagaacccaaaggatgcatctttttggattcctgtacaggagtggtgcagaataacagactaagaaaatatgcctttgaattgaaaatgaatgatctgacctattttgtgctggcagctgaaacagagtcagatatggatgaatggatccacaccctcaaccgcattctgcaaatcagtcctgaggggcccctccaagggaggaggagcacagagctcactgatctgggtctggattcgctggataattctgtaacttgtgaatgcacgccagaggaaacagattcttcagagaacaacctacacgcagactttgcaaagtacctcacagaaacagaagatactgtaaaaacaactcgaaacatggagaggctaaatctgttctctctagatccagacatagataccttgaaacttcaaaaaaaagatctcttggaacctgagtctgtgatcaaaccatttgaagaaaaagctgccaagagaatcatgatcatctgtaaagccctcaactcaaatcttcagggatgtgttacggagaatgaaaatgatccgataacgaatattgagcctttttttgtgagtgtggcactttatgacctcagagacagcaggaagatttctgctgattttcatgtggatctaaaccatgctgctgtcagacagatgctcttgggggcttctgtggctttggaaaatggcaacatcgacaccatcactccaagacaatcagaagaacctcacatcaagggacttccagaggaatggctaaaatttccaaagcaggctgtattttctgtaagcaatccacattctgaaattgttttggtggccaaaatcgaaaaagtcttgatgggaaacattgcaagtggtgccgaaccttatattaagaacccagactccaacaagtatgcacaaaagatactaaaatccaacagacaattctgcagcaaattgggaaaataccgtatgccttttgcttgggcagtaagatcagtatttaaggacaaccagggaaatgtggacagagactcaagattttcaccattgtttagacaagaaagtagcaagatttcaactgaggacctagttaaactagtatcagattatagaagggccgacagaataagcaaaatgcagaccattcctggaagcctggatattgctgttgacaacgttcccttggagcatccaaattgtgtaacatcgtcctttatccctgtcaagcctttcaacatgatggctcaaacagaacccacagtggaggtggaagaatttgtttacgattcaacaaagtattgtcggccttacagagtatataaaaatcaaatttatatttaccccaaacacctcaagtatgatagccagaaatgcttcaacaaggcacggaatataactgtgtgcattgaattcaaaaattcagatgaagaaagtgccaagcccctgaagaacaccccagccggccctcgtatggtctacgcagaactgccgcatactggcccaatagctaatgtgggggacccgtgggatgctcacattatggaaggtgcacgtggccaagtgcctgaggaactggaaaaatggctctgggaatgtatttatggaaaacctggagggcccctcttcacctcagccgcctacacagcagttctgcaccactctcagaatccggatttctcagatgaggtgaaaattgagctaccaacacaactccatgagaaacaccatattttgttttctttttatcacgtcacctgtgacatcaatgcaaaagctaatgccaaaaagaaggaggctctggaaacgtcagttggatatgcttggcttcctctgatgaaacacgatcagatagcttctcaagagtacaacatcccaatagcaacaagtctgcctcctaattatttaagctttcaagattctgcaagtggaaagcatggtgggagtgacattaaatgggttgatggtggcaaaccacttttcaaagtgtcgacatttgttgtatcaacagtaaatactcaggatccacatgtgaatgcatttttccaagagtgccaaaaaagagagaaagatatgtctcagtcacctacctcaaatttcatccgctcttgtaagaacttattgaatgtggaaaagattcatgcaatcatgagttttctgcctataattttgaatcagctcttcaaagttctggtacagaatgaggaagatgaaataactacaactgtcaccagggttctgaccgacattgtggccaagtgccatgaggagcagctggatcattctgtccagtcatatattaagttcgtgttcaagaccagggcatgcaaggagaggactgtacatgaggaactggctaaaaatgtgactggtcttttgaaatcaaatgactcaacaacagtaaagcatgtcctaaagcattcctggttcttctttgcaattatcctaaaatcgatggcacagcacttgattgacacaaataaaatccagcttccccggcctcagagatttcctgaatcttaccaaaatgaattggacaatcttgtcatggtcctatccgaccatgtgatttggaaatacaaggatgcacttgaagaaacaagaagggcaaaccacagcgttgccagatttctcaagcgctgctttacatttatggaccgagggtatgtgtttaagatggtcaacaattacatcagcatgttctcctccggtgaccttaagaccttgtgccagtataaatttgattttcttcaagaagtatgtcaacatgaacactttatccctttgtgtctgcccataagatcagcaaacattccagatcctttgacaccttcagaatcgactcaagagttacatgcatcagatatgcctgaatattcagtcacaaatgaattttgtcgcaaacacttcttaatcggaattctgctccgagaagttggctttgccctgcaggaagaccaagatgtcagacacttagctttagctgtcctaaaaaatctaatggctaagcattcatttgatgatcgatacagagagccaagaaagcaggcccagatagcaagtttatacatgcccctgtacggcatgctcctggacaatatgccaaggatttatctgaaggacctgtatccttttactgtcaatacatctaatcaggggtctagagatgatctaagcaccaatggaggatttcaaagccagacagctatcaaacatgcaaactctgtggatacatcattttctaaagatgttttaaattccatagcagcattttcatcaatagctatttctacagtaaaccatgctgactccagagcatctttagcaagtcttgactccaatccaagtaccaatgagaagagcagtgagaagacggacaactgtgaaaagatcccaagacccttgtctttgattggctcaactcttcgatttgacaagttagatcaagcagaaaccaggagtctcctgatgtgttttcttcacattatgaaaacgatttcgtacgagactctgattgcctactggcagagagctcccagcccagaggtgtccgacttcttcagcatcttggacgtttgtcttcaaaatttcagatacctaggaaaacgcaacataataagaaaaattgctgctgcatttaaatttgtgcagtccacccagaacaatggaactctcaaaggatccaatccttcctgccagacatcaggtctcttgtcacaatggatgcactccacttccagtcatgaaggccataagcagcacagatcacaaactttacctataattcgaggcaaaaatgcactttctaaccccaaactcttacagatgttagacaataccatgaccagcaactccaatgaaatagacatcgtgcatcatgtagacactgaggccaatatagctacggaggtttgcctcactattctggacctgttatccctcttcacacagactcatcagagacaactccaacaatgtgactgtcaaaattcattgatgaaaagggtctttgatacctacatgctctttttccaagtcaatcagtcagccacagcgctgaagcatgtgtttgcctccttgagactgtttgtatgcaagtttccttcagcgttctttcaagggcctgctgacctctgtggatcattctgttacgaagtcctaaaatgctgtaaccacaggtcacggtcaactcagacagaagcctcagcccttctgtactttttcatgaggaagaattttgaatttaacaagcagaagtcaattgtccggtcccacttacaactcatcaaagctgtgagccagttaatagccgatgctgggattggaggctctcggtttcaacattcgcttgcaattaccaataatttcgccaatggagataagcaaatgaaaaacagcaatttcccagcagaggtgaaggacctgactaagcgtataaggactgttttgatggccacagctcagatgaaggagcacgagaaggaccccgagatgctggtggatctccagtacagcctggcaaactcctacgcaagcactcctgaactacgcaggacctggctggaaagtatggccaagattcatgccagaaacggagatttatctgaggctgccatgtgttacatccatattgctgctctcattgcagagtatctgaaaagaaagggttactggaaagtggaaaagatttgcacagcatccctgctctcggaggatacccacccctgtgatagcaactcattactaacaactcccagtggaggaagcatgttctctatgggatggccagcttttttgagcattacaccaaacattaaggaagaaggagcgatgaaagaggattctggaatgcaagatacaccatacaatgagaatatcctggtggagcagctatacatgtgtgtggagtttctctggaagtctgagcgatatgaactcattgctgatgtcaacaagcccatcattgctgtctttgagaaacaacgagacttcaaaaaattgtcagatctctactacgacattcatcggtcatatctgaaagtggcagaggtggtgaattcggagaagcggctgtttggtcgctactatcgtgtggcattttatgggcagggcttttttgaagaagaagaaggtaaagagtatatttataaagagcctaagctgacaggtctgtccgagatttcccaaagattactcaagctctatgcagataaatttggagcagacaatgtgaagataatccaggattccaacaaggtaaaccccaaggatttggaccccaaatatgcctacatccaggtgacctatgtgacgccgttctttgaggaaaaggaaatcgaagaccggaagacagatttcgaaatgcaccacaacatcaaccgctttgtcttcgagacacccttcacgctgtcgggcaagaagcacggtggggtggcggagcagtgcaagcggcggacgatcctgacaacgagtcacctgttcccctacgtgaagaagagaatacaagtaattagccaatcgagcacagaactgaatccaattgaagtggcaattgacgagatgtccaagaaggtttctgagcttaatcagctttgcacaatggaagaagtggacatgatcagactgcagctcaaactgcaaggaagtgtcagcgtgaaggttaatgctgggccaatggcctatgcacgagcttttcttgaagaaaccaatgcaaagaagtaccctgacaaccaagtaaagcttttgaaggagatcttcaggcaatttgcagatgcatgtgggcaggcccttgacgtgaatgagcgcctcatcaaagaggaccagctggagtaccaggaagaactgaggtcccactacaaggacatgctcagcgaactctccacagtcatgaatgagcagattacgggcagggacgacctgtcaaagcgcggagtggaccaaacctgcactcgagtaattagcaaagcaactccggccctacccacggtctccatctcatctagtgctgaagtctga

SEQ ID NO: 9 - protein, 2429 aa, Homo sapiens
MAGERTRRFTRSLLRPGQAAELRHSAASAAAVAVSSRQQQRVSAARPPRLPARPRRPGGTEPSFPKSSAGPGPRSSRVLSSALPPPPPLRAPPAPPQRGSATDAKQTSALRERDAVRGPRPVLGDFRGKSLGQQPASDARILSIPTQMFLRCSRSTLPGRGGKRQINSSGNGKRVGTTASQQDRGSAAALLACVWWTGIRRSCPHICRRRQEEPSHEILPLESDDEMNRPRTLIIMQEKPRLLEPLDYETVIEELEKTYRNDPLQDLLFFPSDDFSAATVSWDIRTLYSTVPEDAEHKAENLLVKEACKFYSSQWHVVNYKYEQYSGDIRQLPRAEYKPEKLPSHSFEIDHEDADKDEDTTSHSSSKGGGGAGGTGVFKSGWLYKGNFNSTVNNTVTVRSFKKRYFQLTQLPDNSYIMNFYKDEKISKEPKGCIFLDSCTGVVQNNRLRKYAFELKMNDLTYFVLAAETESDMDEWIHTLNRILQISPEGPLQGRRSTELTDLGLDSLDNSVTCECTPEETDSSENNLHADFAKYLTETEDTVKTTRNMERLNLFSLDPDIDTLKLQKKDLLEPESVIKPFEEKAAKRIMIICKALNSNLQGCVTENENDPITNIEPFFVSVALYDLRDSRKISADFHVDLNHAAVRQMLLGASVALENGNIDTITPRQSEEPHIKGLPEEWLKFPKQAVFSVSNPHSEIVLVAKIEKVLMGNIASGAEPYIKNPDSNKYAQKILKSNRQFCSKLGKYRMPFAWAVRSVFKDNQGNVDRDSRFSPLFRQESSKISTEDLVKLVSDYRRADRISKMQTIPGSLDIAVDNVPLEHPNCVTSSFIPVKPFNMMAQTEPTVEVEEFVYDSTKYCRPYRVYKNQIYIYPKHLKYDSQKCFNKARNITVCIEFKNSDEESAKPLKNTPAGPRMVYAELPHTGPIANVGDPWDAHIMEGARGQVPEELEKWLWECIYGKPGGPLFTSAAYTAVLHHSQNPDFSDEVKIELPTQLHEKHHILFSFYHVTCDINAKANAKKKEALETSVGYAWLPLMKHDQIASQEYNIPIATSLPPNYLSFQDSASGKHGGSDIKWVDGGKPLFKVSTFVVSTVNTQDPHVNAFFQECQKREKDMSQSPTSNFIRSCKNLLNVEKIHAIMSFLPIILNQLFKVLVQNEEDEITTTVTRVLTDIVAKCHEEQLDHSVQSYIKFVFKTRACKERTVHEELAKNVTGLLKSNDSTTVKHVLKHSWFFFAIILKSMAQHLIDTNKIQLPRPQRFPESYQNELDNLVMVLSDHVIWKYKDALEETRRANHSVARFLKRCFTFMDRGYVFKMVNNYISMFSSGDLKTLCQYKFDFLQEVCQHEHFIPLCLPIRSANIPDPLTPSESTQELHASDMPEYSVTNEFCRKHFLIGILLREVGFALQEDQDVRHLALAVLKNLMAKHSFDDRYREPRKQAQIASLYMPLYGMLLDNMPRIYLKDLYPFTVNTSNQGSRDDLSTNGGFQSQTAIKHANSVDTSFSKDVLNSIAAFSSIAISTVNHADSRASLASLDSNPSTNEKSSEKTDNCEKIPRPLSLIGSTLRFDKLDQAETRSLLMCFLHIMKTISYETLIAYWQRAPSPEVSDFFSILDVCLQNFRYLGKRNIIRKIAAAFKFVQSTQNNGTLKGSNPSCQTSGLLSQWMHSTSSHEGHKQHRSQTLPIIRGKNALSNPKLLQMLDNTMTSNSNEIDIVHHVDTEANIATEVCLTILDLLSLFTQTHQRQLQQCDCQNSLMKRVFDTYMLFFQVNQSATALKHVFASLRLFVCKFPSAFFQGPADLCGSFCYEVLKCCNHRSRSTQTEASALLYFFMRKNFEFNKQKSIVRSHLQLIKAVSQLIADAGIGGSRFQHSLAITNNFANGDKQMKNSNFPAEVKDLTKRIRTVLMATAQMKEHEKDPEMLVDLQYSLANSYASTPELRRTWLESMAKIHARNGDLSEAAMCYIHIAALIAEYLKRKGYWKVEKICTASLLSEDTHPCDSNSLLTTPSGGSMFSMGWPAFLSITPNIKEEGAMKEDSGMQDTPYNENILVEQLYMCVEFLWKSERYELIADVNKPIIAVFEKQRDFKKLSDLYYDIHRSYLKVAEVVNSEKRLFGRYYRVAFYGQGFFEEEEGKEYIYKEPKLTGLSEISQRLLKLYADKFGADNVKIIQDSNKVNPKDLDPKYAYIQVTYVTPFFEEKEIEDRKTDFEMHHNINRFVFETPFTLSGKKHGGVAEQCKRRTILTTSHLFPYVKKRIQVISQSSTELNPIEVAIDEMSKKVSELNQLCTMEEVDMIRLQLKLQGSVSVKVNAGPMAYARAFLEETNAKKYPDNQVKLLKEIFRQFADACGQALDVNERLIKEDQLEYQEELRSHYKDMLSELSTVMNEQITGRDDLSKRGVDQTCTRVISKATPALPTVSISSSAEV

SEQ ID NO: 10 - DNA, 1680 bp, Homo sapiens
caggccgcgagcccggagcgcgccggctggaccgaggcgctgcgggccgccgtggccgagctgcgcgccggcgccgtggtggccgtccccaccgatacgctgtacggcctggcctgcgcggcgagctgctcggcggctctgcgcgctgtgtaccgcctcaagggtcgcagcgaggccaagcctctggccgtatgcctcggccgcgtggccgacgtctacagatactgccgtgtgagagtacctgaggggctcctgaaagacctactgccaggaccagtgaccctggtgatggaacgctcggaggagctcaacaaggacctaaacccttttacgcctcttgtaggcattcggattcctgatcatgcttttatgcaagacttggctcagatgtttgagggtccgcttgctctcactagtgccaacctcagctcccaggccagttctctgaatgtcgaggagttccaggatctctggcctcagttgtccttggttattgatgggggacaaattggggatggccagagccccgagtgtcgccttggctcaactgtggttgatttgtctgtgcccggaaagtttggcatcattcgtccaggctgtgccctggaaagtactacagccatcctccaacagaagtacggactgctcccctcacatgcgtcctacctgtgaaactctgggaagcaggaaggcccaagacctggtgctggatactatgtgtctgtccactgacgactgtcaaggcctcatttgcagaggccaccggagctagggcactagcctgacttttaaggcagtgtgtctttctgagcactgtagaccaagcccttggagctgctggtttagccttgcacctggggaaaggatgtatttatttgtattttcatatatcagccaaaagctgaatggaaaagttaagaacattcctaggtggccttattctaataagtttcttctgtctgttttgtttttcaattgaaaagtaattaaataacagatttagaatctagtgagagcctcctctctggtgggtggtggcatttaaggttcaaaccagccagaagtgctggtgctgtttaaaaagtctcaggtggctgcgtgtggtggctcatgcctgtaatcccaacattctgggaggcccaggcgggagaactgcttgagcccaggagttcagaatcagcctgggcaacatagcaatactccgtctcataaaaattaataaataaaaagtctcaggtgaccaaaggctcctgaagctagaaccaggtttggataaagattgaagagccacaggccactcttccctctgagccattgggcctagtggtgtcatgtattgtaattgctcgcagggagagcagtctttttggtgtaatagtgggatgtctgcttagttggcaggggttcagtccaaatggaagaatattgggaaataaacctccactatcctttatagccagggacttttttcctatttattcataaaataaattatagttaattatacccataacacctttatttaaatccagtgttctccgcagccttttgtctatttatatgtgtaccaagtgttaaacataattattattgggcatttgaactttgtttttctttaaagaaatgctgctattaaacatatttgtaaatgraaaaaaaaaaaaaaaaaaaaaaaaaaaa

SEQ ID NO: 11 - protein, 223 aa, Homo sapiens
QAASPERAGWTEALRAAVAELRAGAVVAVPTDTLYGLACAASCSAALRAVYRLKGRSEAKPLAVCLGRVADVYRYCRVRVPEGLLKDLLPGPVTLVMERSEELNKDLNPFTPLVGIRIPDHAFMQDLAQMFEGPLALTSANLSSQASSLNVEEFQDLWPQLSLVIDGGQIGDGQSPECRLGSTVVDLSVPGKFGIIRPGCALESTTAILQQKYGLLPSHASYL

SEQ ID NO: 12 - DNA, 1850 bp, Homo sapiens
  misc_feature (1179)..(1179): n is a, c, g, or t
ccggcgcgtcggtgcgggggatgagggccgcggtggctgccagcgtggggttgagcgaggggcctgctggctcccggagcggtcgcctcttccgcccgccgagtcccgctccggcggcccccggcgcccggctgttgcggctcccggggagcggggccgtgcaggccgcgagcccggagcgcgccggctggaccgaggcgctgcgggccgccgtggccgagctgcgcgccggcgccgtggtggccgtccccaccgatacgctgtacggcctggcctgcgcggcgagctgctcggcggctctgcgcgctgtgtaccgcctcaagggtcgcagcgaggccaagcctctggccgtatgcctcggccgcgtggccgacgtctacagatactgccgtgtgagagtacctgaggggctcctgaaagacctactgccaggaccagtgaccctggtgatggaacgctcggaggagctcaacaaggacctaaacccttttacgcctcttgtaggcattcggattcctgatcatgcttttatgcaagacttggctcagatgtttgagggtccgcttgctctcactagtgccaacctcagctcccaggccagttctctgaatgtcgaggagttccaggatctctggcctcagttgtccttggttattgatgggggacaaattggggatggccagagccccgagtgtcgccttggctcaactgtggttgatttgtctgtgcccggaaagtttggcatcattcgtccaggctgtgccctggaaagtactacagccatcctccaacagaagtacggactgctcccctcacatgcgtcctacctgtgaaactctgggaagcaggaaggcccaagacctggtgctggatactatgtgtctgtccactgacgactgtcaaggcctcatttgcagaggccaccggagctagggcactagcctgacttttaaggcagtgtgtctttctgagcactgtagaccaagcccttggagctgctggtttagccttgcacctggggaaaggatgtatttatttgtattttcatatatcagccaaaagctgaatggaaaagttaagaacattcctaggtggccttattctaataagtttcttctgtctgttttgtttttcaattgaaaagtaattaaataacagatttagaatctagtgagagcntcctctctgggggtggtggcatttaaggttcaacccanccnagaagtgctgcgctgtttaaaaagtctcaggtggctgcgtgtggtggctcatgcctgtaatcccaacattctgggaggcccaggcgggagaactgcttgagcccaggagttcagaatcagcctgggcaacatagcaatactccgtctcataaaaattaataaataaaaagtctcaggtgaccaaaggctcctgaagctagaaccaggtttggataaagattgaagagccacaggccactcttccctctgagccattgggcctagtggtgtcatgtattgtaattgctcgcagggagagcagtctttttggtgtaatagtgggatgtctgcttagttggcaggggttcagtccaaatggaagaatattgggaaataaacctcctctatcctttatagccagggacttttttcttatttattcataaaataaattatagttaattatacccataacacctttatttaaatccagtgttctccgcagccttttgtctatttatatgtgtaccaagtgttaaacataattattattgggcatttgaactttgtttttctttaaagaaatgctgctattaaacatatttgtaaatggtttaaaaaaaaaaaaaaaaaaaaaaaaaaaaaaaaaaa

SEQ ID NO: 13 - protein, 270 aa, Homo sapiens
MRAAVAASVGLSEGPAGSRSGRLFRPPSPAPAAPGARLLRLPGSGAVQAASPERAGWTEALRAAVAELRAGAVVAVPTDTLYGLACAASCSAALRAVYRLKGRSEAKPLAVCLGRVADVYRYCRVRVPEGLLKDLLPGPVTLVMERSEELNKDLNPFTPLVGIRIPDHAFMQDLAQMFEGPLALTSANLSSQASSLNVEEFQDLWPQLSLVIDGGQIGDGQSPECRLGSTVVDLSVPGKFGIIRPGCALESTTAILQQKYGLLPSHASYL

SEQ ID NO: 14 - protein, 100 aa, Homo sapiens
MATFISVQLKKTSEVDLAKPLVKFIQQTYPSGGEEQAQYCRAAEELSKLRRAAVGRPLDKHEGALETLLRYYDQICSIEPKFPFSENQICLTFTWKDAFD

SEQ ID NO: 15 - DNA, 3182 bp, Homo sapiens
gccagtccgccagcccagtacctctctctcctcggccctcgtaagctgtccgcggtctgtttggcccgaacggcggcggaggcgctgatcatggcgacattcatctcggtgcagctgaaaaagacctcagaggtggacctggccaagccgctggtgaagttcatccagcagacttacccaagcggcggggaagagcaggcccagtactgccgcgcggcggaggagctcagcaagctgcgccgcgccgcagtcggtcgtccgctggacaagcacgagggcgcgctcgagacgctcctgagatattatgatcagatttgttctattgaacccaaattcccattttctgaaaatcagatctgcttgacatttacctggaaggatgctttcgataaaggttcactttttggaggctctgtaaaactggctcttgcaagcttaggatatgaaaagagctgtgtgttgttcaattgtgcagccttagctagccaaattgcagcagaacagaacctggataatgatgaaggattgaaaatcgctgctaaacattaccagtttgctagtggtgcctttttacatattaaagagacggttttatctgccttaagtcgagagccgaccgtggacatatctccagatactgttgggaccctcagtcttattatgctggcacaggctcaagaagtattttttttaaaagccacaagagataaaatgaaagatgccatcatagctaaattggctaatcaggctgcagattattttggtgatgctttcaaacagtgtcaatacaaagatactctccccaaggaggtgttccctgtcttggctgcaaagcactgtatcatgcaggccaatgctgagtaccatcagtctatcctggcaaaacagcagaagaaatttggagaagaaattgcaaggttacagcatgcagcagaactgattaaaacagtggcatctcgctatgatgaatatgttaatgtgaaggatttttctgacaaaatcaatcgtgcccttactgcagcaaagaaggataatgacttcatttatcatgatcgagttccagaccttaaagatctagatcctattggcaaagccacacttgtgaaatctaccccggtcaatgtacccatcagtcagaaatttactgatctgtttgagaagatggttcccgtgtcagtacagcagtctttggctgcctataatcagaggaaagccgatttggttaacagatcaattgctcagatgagagaagccaccactttggcaaatggggtgctagcttcccttaatcttccagcagcaattgaagatgtgtctggagacactgtacctcagtctatattgactaaatccagatctgtgattgaacagggaggcatccagactgttgatcagttgattaaagaactgcctgaattactgcaacgaaatagagaaatcctagatgagtcattaaggttgttggatgaagaagaagcaaccgataatgatttaagagcaaaatttaaggaacgttggcaaaggacaccatccaatgaactgtataagcctttaagagcagagggaaccaacttcagaacagttttagataaagctgtgcaggcagatggacaagtgaaagaatgttaccagtctcatcgtgacaccatcgtgcttttatgtaagccagagcctgagctgaatgctgccatcccttctgctaatccagcaaagaccatgcagggcagtgaggttgtaaatgtcttaaaatccttattgtcaaatcttgatgaagtaaagaaggaaagagagggtctggagaatgacttgaaatctgtgaattttgacatgacaagcaagtttttgacagccctggctcaagatggtgtgataaatgaagaagctctttctgttactgaactagatcgagtctatggaggtcttacaactaaagtccaagaatctctaaagaaacaggagggacttcttaaaaatattcaggtctcacatcaggaattttcgaaaatgaaacaatctaataatgaagctaacttaagagaagaagttttgaagaatttagctactgcatatgacaactttgttgaacttgtagctaatttgaaggaaggcacaaagttttacaatgagttgactgaaatcctggtcaggttccagaacaaatgcagtgatatagtttttgcacggaagacagaaagagatgaactcttaaaggacttgcaacaaagcattgccagagaacctagtgctccttcaattcctacacctgcgtatcagtccttaccagcaggaggacatgcaccaactcctccaactccagcgccaagaaccatgccgcctactaagccccagcccccagccaggcctccaccacctgtgcttccagcaaatcgagctccttctgctactgctccatctccagtgggggctgggactgctgcgccagctccatcacaaacgcctggctcagctcctcctccacaggcgcagggaccaccctatcccacctatccaggatatcctgggtattgccaaatgcccatgcccatgggctataatccttatgcgtatggccagtataatatgccatatccaccagtgtatcaccagagtcctggacaggctccgtacccgggaccccagcagccttcataccccttccctcagcccccacagcagtcttactatccacagcagtaatatgtctgctcagcagctcagctgattcagatcagagggaaagaaataccaaccctgcaataagtgtactaaactctacgctctggttaatgtaatgtactctcctggactgaatgcagtgtataatttctgtctacagctagaagctgtgccccagttccacatttgattacacatgtgagatttgctgctgttgcagtataaacactaggtataataggatttgaaattgcattacagttcataaaaattgaaaatgagaaattaaacctgcaagtgaaacatttgaaacgattatactttctacataagacatggttgggacatcagatacttacaaagatggtttaagtatggatactagagaaaattaagttttctttctctttggtttattgatttggtttaatttccattatgctattttgcataatcaaggcactgtaaatcttataattttaaaataaattacttaagaacaagtg

SEQ ID NO: 16 - protein, 868 aa, Homo sapiens [sequence omitted]

SEQ ID NO: 17 - DNA, 2093 bp, Homo sapiens
gtcccaggcgctcgcgagggggtagctcttctagtagtgctcggcgtcagacatggcggaggcgatggatttgggcaaagaccccaacgggcccacccattcctcgactctgttcgtgagggacgacggcagctccatgtccttctacgtgcggcccagcccggccaagcgtcggctgtcgacgctcatcctgcacggcggcggcaccgtgtgccgagtgcaggagcccggggccgtgctgctggcccagcccggggaggcgctggccgaggcctcgggtgatttcatctccacgcagtacatcctggactgcgtggagcgcaacgagaggctggagctggaggcctatcggctgggccccgcctcggcggcggacaccggctcggaagcaaagcccggggccctggccgagggcgccgcggagccggagccgcagcggcacgccgggcggatcgccttcacggatgcggacgacgtagccatccttacctacgtgaaggaaaatgcccgctcgcccagctccgtcaccggtaacgccttgtggaaagcgatggagaagagctcgctcacgcagcactcgtggcagtccctgaaggaccgctacctcaagcacctgcggggccaggagcataagtacctgctgggggacgcgccggtgagcccctcctcccagaagctcaagcggaaggcggaggaggacccggaggccgcggatagcggggaaccacagaataagagaactccagatttgcctgaagaagagtatgtgaaggaagaaatccaggagaatgaagaagcagtcaaaaagatgcttgtggaagccacccgggagtttgaggaggttgtggtggatgagagccctcctgattttgaaatacatataactatgtgtgatgatgatccacccacacctgaggaagactcagaaacacagcctgatgaggaggaagaagaagaagaagaaaaagtttctcaaccagaggtgggagctgccattaagatcattcggcagttaatggagaagtttaacttggatctatcaacagttacacaggccttcctaaaaaatagtggtgagctggaggctacttccgccttcttagcgtctggtcagagagctgatggatatcccatttggtcccgacaagatgacatagatttgcaaaaagatgatgaggataccagagaggcattggtcaaaaaatttggtgctcagaatgtagctcggaggattgaatttcgaaagaaataattggcaagataatgagaaaagaaaaaagtcatggtaggtgaggtggttaaaaaaaattgtgaccaatgaactttagagagttcttgcattggaactggcacttattttctgaccatcgctgctgttgctctgtgagtcctagatttttgtagccaagcagagttgtagagggggataaaaagaaaagaaattggatgtatttacagctgtccttgaacaagtatcaatgtgtttatgaaaggaagatctaaatcagacaggagttggtctacatagtagtaatccattgttggaatggaacccttgctatagtagtgacaaagtgaaaggaaatttaggaggcataggccatttcaggcagcataagtaatctcctgtcctttggcagaagctcctttagattgggatagattccaaataaagaatctagaaataggagaagatttaattatgaggccttgaacacggattatccccaaacccttgtcatttcccccagtgagctctgatttctagactgctttgaaaatgctgtattcattttgctaacttagtatttgggtaccctgctctttggctgttctttttttggagcccttctcagtcaagtctgccggatgtctttctttacctacccctcagttttccttaaaacgcgcacacaactctagagagtgttaagaataatgttacttggttaatgtgttatttattgagtattgtttgtgctaagcattgtgttagatttaaaaaattagtggattgactccactttgttgtgttgttttcattgttgaaaataaatataactttgtattcgaaaaaaaaaaaaaaaaaaaag

SEQ ID NO: 18 - protein, 399 aa, Homo sapiens
MAEAMDLGKDPNGPTHSSTLFVRDDGSSMSFYVRPSPAKRRLSTLILHGGGTVCRVQEPGAVLLAQPGEALAEASGDFISTQYILDCVERNERLELEAYRLGPASAADTGSEAKPGALAEGAAEPEPQRHAGRIAFTDADDVAILTYVKENARSPSSVTGNALWKAMEKSSLTQHSWQSLKDRYLKHLRGQEHKYLLGDAPVSPSSQKLKRKAEEDPEAADSGEPQNKRTPDLPEEEYVKEEIQENEEAVKKMLVEATREFEEVVVDESPPDFEIHITMCDDDPPTPEEDSETQPDEEEEEEEEKVSQPEVGAAIKIIRQLMEKFNLDLSTVTQAFLKNSGELEATSAFLASGQRADGYPIWSRQDDIDLQKDDEDTREALVKKFGAQNVARRIEFRKK

SEQ ID NO: 19 - DNA, 2304 bp, Homo sapiens
gaattcgtccaaactgaggatcacaagtctccacattctgagtaggaggatgagggtctgagttaggatttgggtcctgcagggcttgctaaggaatcccctgatggcctaggattccacgcagagcacatctggtgtgagagagctcgctgcaagggtgaaggctccgccctatcagatagacaaccaggccaccaagaggcccagccctccaaaccctggatttgcaacatcctcaaagaacagcaacgggccttgagcagaattgagaaggaaatacccccacctgccctcagccgttaagtgggctttgctattcacaagggcctctgggtgtcctggcagagaggggagatggcacaggcaccaggtgctagggtgccagggcctcccgagaaggaacaggtgcaaagcaggcaattagcccagaaggtatccgtggggcaggcagcctagatctgatgggggaagccaccaggattacatcatctgctgtaacaactgctctgaaaagaagatatttttcaacctgaacttgcagtagctagtggagaggcaggaaaaaggaaatgaaacagagacagagggaagcctgagccaaaatagaccttcccgagagaggaggaagcccggagagagacgcacggtcccctccccgcccctaggccgccgccccctctctgccctcggcggcgagcagggcgccgcgacccggggccggaaaggtgccaggggctccgggcggccgggcgggcgcacaccatccccgcgggcggcgcggagccggcgacagcgcgcgagagggaccgggcggtggcggcggcgggaccgggatggaagggagcgcggtgactgtccttgagcgcggaggggcgagctcgccggcggaggccgagcaagcggaggcaggagcggcggcgacggcggcggcggcggcggcgcccgagcacccgagggggtccgagccccggcagccggccagccccgcgccacaaagggagcgcccccgccgcccggcaccccgcctccctccccaatgtcctcggccatcgaaaggaagagcctggacccttcagaggaaccagtggatgaggtgctgcagatccccccatccctgctgacatgcggcggctgccagcagaacatcggggaccgctacttcctgaaggccatcgaccagtactggcacgaggactgcctgagctgcgacctctgtggctgccggctgggtgaggtggggcggcgcctctactacaaactgggccggaagctctgccggagagactatctcaggctttttgggcaagacggtctctgcgcatcctgtgacaagcggattcgtgcctatgagatgacaatgcgggtgaaagacaaagtgtatcacctggaatgtttcaagtgcgccgcctgtcagaagcatttctgtgtaggtgacagatacctcctcatcaactctgacatagtgtgcgaacaggacatctacgagtggactaagatcaatgggatgatataggcccgagtccccgggcatctttggggaggtgttcactgaagacgccgtctccatggcatcttcgtcttcactcttaggcactttgggggtttgagggtggggtaagggatttcttaggggatggtagacctttattgggtatcaagacatagcatccaagtggcataattcaggggctgacacttcaaggtgacagaaggaccagcccttgagggagaacttatggccacagcccatccatagtaactgacatgattagcagaagaaaggaacatttaggggcaagcaggcgctgtgctatcatgatggaatttcatatctacagatagagagttgttgtgtacagacttgttgtgactttgacgcttgcgaactagagatgtgcaattgatttcttttcttcctggctttttaactcccctgtttcaatcactgtcctccacacaagggaaggacagaaaggagagtggccattctttttttcttggcccccttcccaaggccttaagctttggacccaagggaaaactgcatggagacgcatttcggttgagaatggaaaccacaacttttaaccaaacaattatttaaagcaatgctgatgaatcactgtttttagacaccttcattttgaggggaggagttccacagattgtttctatacaaatataaatcttaaaaagttgttcaactattttattatcctagattatatcaaagtatttgtcgtgtgtagaaaaaaaaaacagctctgcaggcttaataaaaatgacagactgaaaaaaaaaaaa

SEQ ID NO: 20 - protein, 158 aa, Homo sapiens
MSSAIERKSLDPSEEPVDEVLQIPPSLLTCGGCQQNIGDRYFLKAIDQYWHEDCLSCDLCGCRLGEVGRRLYYKLGRKLCRRDYLRLFGQDGLCASCDKRIRAYEMTMRVKDKVYHLECFKCAACQKHFCVGDRYLLINSDIVCEQDIYEWTKINGMI

SEQ ID NO: 21 - DNA, 1075 bp, Homo sapiens
ctgcaaggcggcggcaggagaggttgtggtgctagtttctctaagccatccagtgccatcctcgtcgctgcagcgacaccgctctcgccgccgccatgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacttccctggcctggtctgctgatggccagactctgtttgctggctacacggacaacctggtgcgagtgtggcaggtgaccattggcacacgctagaagtttatggcagagctttacaaata

SEQ ID NO: 22 - protein, 317 aa, Homo sapiens [sequence omitted]

SEQ ID NO: 23 - DNA, 1486 bp, Homo sapiens
caaatcttccaaagaatcctctaagaaacccaaagaaaataaaccactgaaagaagagaaaatagttcctaagatggccttcaaggaacctaaacccatgtcaaaagagccaaaaccagatagtaacttactcaccatcaccagtggacaagataagaaggctcctagtaaaaggccgcccatttcagattctgaagaactctcagccaaaaaaaggaaaaagagtagctcagaggctttatttaaaagtttttctagcgcaccaccactgatactcacttgttctgctgacaaaaaacagataaaagataaatctcatgtcaagatgggaaaggtcaaaattgaaagtgagacatcagagaagaagaaatcaacgttaccgccatttgatgatattgtggatcccaatgattcagatgtggaggagaatatatcctctaaatctgattctgaacaacccagtcctgccagctccagctccagctccagctccagcttcacaccatcccagaccaggcaacaaggtcctttgaggtctataatgaaagatctgcattctgatgacaatgaggaggaatcagatgaagtggaggataacgacaatgactctgaaatggagaggcctgtaaatagaggaggcagccgaagtcgcagagttagcttaagtgatggcagcgatagtgaaagcagttctgcttcttcacccctacatcacgaacctccaccacccttactaaaaaccaacaacaaccagattcttgaagtgaaaagtccaataaagcaaagcaaatcagataagcaaataaagaatggtgaatgtgacaaggcatacctagatgaactggtagagcttcacagaaggttaatgacattgagagaaagacacattctgcagcagatcgtgaaccttatagaagaaactggacactttcatatcacaaacacaacatttgattttgatctttgctcgctggacaaaaccacagtccgtaaactacagagttacctggaaacatctggaacatcctgaggatataacaactggatgcatcaagaactattgtgttttttttttttggttttttttttttttggttgtgattttttgttcttgttgtttatatgaaaacactcaaaatgatgcaaccaaaagggaaaaaataaaaatcaaacaaccttcagctttatttttctttaaagccagtcatcatctcttgataaaggagaggttaaagcaaaccagcctcagcggaccactcttctctccaaggaaatccccgggaagagttagcctggatagccttgaaaacaaacaaatcaaacacaacacaagaaaactcaaagaatgtgtatggtatcatgtatctctctgtggtggttcattccacaggacgaatgcatattcaacacactgccttattacataactgatctatttattatcgcatacagatattctaagtcgttgagggaatgacaccatcag

SEQ ID NO: 24 - protein, 342 aa, Homo sapiens
KSSKESSKKPKENKPLKEEKIVPKMAFKEPKPMSKEPKPDSNLLTITSGQDKKAPSKRPPISDSEELSAKKRKKSSSEALFKSFSSAPPLILTCSADKKQIKDKSHVKMGKVKIESETSEKKKSTLPPFDDIVDPNDSDVEENISSKSDSEQPSPASSSSSSSSSFTPSQTRQQGPLRSIMKDLHSDDNEEESDEVEDNDNDSEMERPVNRGGSRSRRVSLSDGSDSESSSASSPLHHEPPPPLLKTNNNQILEVKSPIKQSKSDKQIKNGECDKAYLDELVELHRRLMTLRERHILQQIVNLIEETGHFHITNTTFDFDLCSLDKTTVRKLQSYLETSGTS

SEQ ID NO: 25 - DNA, 3376 bp, Homo sapiens
tttggggctgagtttaataagcgagcgagcgagcaagcgagcgcggggggaaaaaggcagagaatgtccgccatctaccctccgctcctgggcgcgctctcattcatagcagcctcttcatgaattacagctgagggggggcggaggaggggggggtaccacacaacaccccagcaaacctccgggcccccaggcatggctagctcgtgttccgtgcaggtgaagctggagctggggcaccgcgcccaggtgaggaaaaaacccaccgtggagggcttcacccacgactggatggtgttcgtacgcggtccggagcacagtaacatacagcactttgtggagaaagtcgtcttccacttgcacgaaagctttcctaggccaaaaagagtgtgcaaagatccaccttacaaagtagaagaatctgggtatgctggtttcattttgccaattgaagtttattttaaaaacaaggaagaacctaggaaagtccgctttgattatgacttattcctgcatcttgaaggccatccaccagtgaatcacctccgctgtgaaaagctaactttcaacaaccccacagaggactttaggagaaagttgctgaaggcaggaggggaccctaataggagtattcataccagcagcagcagcagcagcagcagtagcagcagcagcagcagcagcagcagcagcagtagcagcagcagcagcagcagcagcagcagcagtagcagcagcagtagcagcagcagcagcagcagtagtaccagtttttcaaagcctcacaaattaatgaaggagcacaaggaaaaaccttctaaagactccagagaacataaaagtgccttcaaagaaccttccagggatcacaacaaatcttccaaagaatcctctaagaaacccaaagaaaataaaccactgaaagaagagaaaatagttcctaagatggccttcaaggaacctaaacccatgtcaaaagagccaaaaccagatagtaacttactcaccatcaccagtggacaagataagaaggctcctagtaaaaggccgcccatttcagattctgaagaactctcagccaaaaaaaggaaaaagagtagctcagaggctttatttaaaagtttttctagcgcaccaccactgatactcacttgttctgctgacaaaaaacagataaaagataaatctcatgtcaagatgggaaaggtcaaaattgaaagtgagacatcagagaagaagaaatcaacgttaccgccatttgatgatattgtggatcccaatgattcagatgtggaggagaatatatcctctaaatctgattctgaacaacccagtcctgccagctccagctccagctccagctccagcttcacaccatcccagaccaggcaacaaggtcctttgaggtctataatgaaagatctgcattctgatgacaatgaggaggaatcagatgaagtggaggataacgacaatgactctgaaatggagaggcctgtaaatagaggaggcagccgaagtcgcagagttagcttaagtgatggcagcgatagtgaaagcagttctgcttcttcacccctacatcacgaacctccaccacccttactaaaaaccaacaacaaccagattcttgaagtgaaaagtccaataaagcaaagcaaatcagataagcaaataaagaatggtgaatgtgacaaggcatacctagatgaactggtagagcttcacagaaggttaatgacattgagagaaagacacattctgcagcagatcgtgaaccttatagaagaaactggacactttcatatcacaaacacaacatttgattttgatctttgctcgctggacaaaaccacagtccgtaaactacagagttacctggaaacatctggaacatcctgaggatataacaactggatgcatcaagaactattgtgttttttttttttggttttttttttttttggttgtgattttttgttcttgttgtttatatgaaaacactcaaaatgatgcaaccaaaagggaaaaaataaaaatcaaacaaccttcagctttatttttctttaaagccagtcatcatctcttgataaaggagaggttaaagcaaaccagcctcagcggaccactcttctctccaaggaaatccccgggaagagttagcctggatagccttgaaaacaaacaaatcaaacacaacacaagaaaactcaaagaatgtgtatggtatcatgtatctctctgtggtggttcattccacaggacgaatgcatattcaacacactgccttattacataactgatctatttattatcgcatacagatattctaagtcgttgagggaatgacaccatcagacattataagtacttggtcccgtggatgctctttcaatgcagcacccttgccatcccaagcccagtgaccttactcgtataccgtgccactttccaccaactttttccaagtcctttaactcgttgcagtctgtattttccaccttttgtttttccagttccaggacacagattatcaactggggggaccaaatagccaccttgattttcttctttgtggtctttttcctgaaagttggggcccagtccttggctgtatccatgtaatgatcttggaccatggtagaaaatgcaccaaataggatcatatgaattgctgtctagccttagtcaataaacttgtaggacttttaaacaaaagtgtacctgtaaatgtcctgaatccagcattgttgagctgtcatcaacattcttgtgtctgttttactgttacaatattaggtgaatatggaagtaaaggcattccacaggatcatcatttaaaaaaaaagaattctggtcctgttttctaaaaaaaaaaaactgttgtagaaattcttaatttggatctatttattagtcagagtttcagctttcttcagctgccagtgtgttactcatctttatcctaaaaatctggaatcagagatttttgtttgttcacatatgattctcttagacacttttatatttgaaaaaattaaaatctttctttggggaaaaattcttggttattctgccataacagattatgtattaacttgtagattcagtggttcaatacctgtttagttgcttgctaatatttccagaaggatttcttgtattggtgaaagacggttggggatggggggatttttttgttcttgttgtacccttgttttgaaactagaaatctgtcctgtggcatgcaaaagaaagcaaattatttttaaaagaaaaaaaccaaagtacttttggtgtcattattccatcttctccataagtggagaaatgaaaagtaagaacagctcatcttcaaagtttttactagaaattc

SEQ ID NO: 26 - protein, 568 aa, Homo sapiens
MASSCSVQVKLELGHRAQVRKKPTVEGFTHDWMVFVRGPEHSNIQHFVEKVVFHLHESFPRPKRVCKDPPYKVEESGYAGFILPIEVYFKNKEEPRKVRFDYDLFLHLEGHPPVNHLRCEKLTFNNPTEDFRRKLLKAGGDPNRSIHTSSSSSSSSSSSSSSSSSSSSSSSSSSSSSSSSSSSSSSSSSSTSFSKPHKLMKEHKEKPSKDSREHKSAFKEPSRDHNKSSKESSKKPKENKPLKEEKIVPKMAFKEPKPMSKEPKPDSNLLTITSGQDKKAPSKRPPISDSEELSAKKRKKSSSEALFKSFSSAPPLILTCSADKKQIKDKSHVKMGKVKIESETSEKKKSTLPPFDDIVDPNDSDVEENISSKSDSEQPSPASSSSSSSSSFTPSQTRQQGPLRSIMKDLHSDDNEEESDEVEDNDNDSEMERPVNRGGSRSRRVSLSDGSDSESSSASSPLHHEPPPPLLKTNNNQILEVKSPIKQSKSDKQIKNGECDKAYLDELVELHRRLMTLRERHILQQIVNLIEETGHFHITNTTFDFDLCSLDKTTVRKLQSYLETSGTS

SEQ ID NO: 27 - DNA, 2088 bp, Homo sapiens
gaattcggcacgagcgcgcggcgaatctcaacgctgcgccgtctgcgggcgcttccgggccaccagtttctctgctttccaccctggcgccccccagccctggctccccagctgcgctgccccgggcgtccacgccctgcgggcttagcgggttcagtgggctcaatctgcgcagcgccacctccatgttgaccaagcctctacaggggcctcccgcgccccccgggacccccacgccgccgccaggaggcaaggatcgggaagcgttcgaggccgagtatcgactcggccccctcctgggtaaggggggctttggcaccgtcttcgcaggacaccgcctcacagatcgactccaggtggccatcaaagtgattccccggaatcgtgtgctgggctggtcccccttgtcagactcagtcacatgcccactcgaagtcgcactgctatggaaagtgggtgcaggtggtgggcaccctggcgtgatccgcctgcttgactggtttgagacacaggaaggcttcatgctggtcctcgagcggcctttgcccgcccaggatctctttgactatatcacagagaagggcccactgggtgaaggcccaagccgctgcttctttggccaagtagtggcagccatccagcactgccattcccgtggagttgtccatcgtgacatcaaggatgagaacatcctgatagacctacgccgtggctgtgccaaactcattgattttggttctggtgccctgcttcatgatgaaccctacactgactttgatgggacaagggtgtacagccccccagagtggatctctcgacaccagtaccatgcactcccggccactgtctggtcactgggcatcctcctctatgacatggtgtgtggggacattccctttgagagggaccaggagattctggaagctgagctccacttcccagcccatgtctccccagactgctgtgccctaatccgccggtgcctggcccccaaaccttcttcccgaccctcactggaagagatcctgctggacccctggatgcaaacaccagccgaggatgttacccctcaacccctccaaaggaggccctgcccctttggcctggtccttgctaccctaagcctggcctggcctggcctggcccccaatggtcagaagagccatcccatggccatgtcacagggatagatggacatttgttgacttggttttacaggtcattaccagtcattaaagtccagtattactaaggtaagggattgaggatcaggggttagaagacataaaccaagtttgcccagttcccttcccaatcctacaaaggagccttcctcccagaacctgtggtccctgattttggagggggaacttcttgcttctcattttgctaaggaagtttattttggtgaagttgttcccattttgagccccgggactcttattttgatgatgtgtcaccccacattggcacctcctactaccaccacacaaacttagttcatatgcttttacttgggcaagggtgctttccttccaataccccagtagcttttattttagtaaagggaccctttcccctagcctagggtcccatattgggtcaagctgcttacctgcctcagcccaggattttttattttgggggaggtaatgccctgttgttaccccaaggcttctttttttttttttttttttttgggtgaggggaccctactttgttatcccaagtgctcttattctggtgagaagaaccttaattccataatttgggaaggaatggaagatggacaccaccggacaccaccagacaataggatgggatggatggttttttgggggatgggctaggggaaataaggcttgctgtttgttttcctggggcgctccctccaattttgcagatttttgcaacctcctcctgagccgggattgtccaattactaaaatgtaaataatcacgtattgtggggaggggagttccaagtgtgccctccttttttttcctgcctggattatttaaaaagccatgtgtggaaacccactatttaataaaagtaatagaatcagaaaaaaaaaaaaaaaaaaa

SEQ ID NO: 28 - protein, 334 aa, Homo sapiens
MLTKPLQGPPAPPGTPTPPPGGKDREAFEAEYRLGPLLGKGGFGTVFAGHRLTDRLQVAIKVIPRNRVLGWSPLSDSVTCPLEVALLWKVGAGGGHPGVIRLLDWFETQEGFMLVLERPLPAQDLFDYITEKGPLGEGPSRCFFGQVVAAIQHCHSRGVVHRDIKDENILIDLRRGCAKLIDFGSGALLHDEPYTDFDGTRVYSPPEWISRHQYHALPATVWSLGILLYDMVCGDIPFERDQEILEAELHFPAHVSPDCCALIRRCLAPKPSSRPSLEEILLDPWMQTPAEDVTPQPLQRRPCPFGLVLATLSLAWPGLAPNGQKSHPMAMSQG

SEQ ID NO: 29 - DNA, 2672 bp, Homo sapiens
gagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagaccagtactttctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgaggcgagccaccatttcaaacccaataacaggagacttggagacggtacattacagaatttctaaaagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttccatagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattc

SEQ ID NO: 30 - protein, 534 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRPVLSNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLRRATISNPITGDLETVHYRISKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 31 - DNA, 8843 bp, Homo sapiens
agccggcgagcggcagcagctccgggctcgagagcccgcgcccatgccagtgcccatgcggggccgccgccagtgacgccggagaggtgttccccccacactggggctcccactactgcgaggagtgacccacgaaggccacagagatggccggggcttcggtgaaggtggcggtgcgggtccgccccttcaattcccgggaaatgagccgtgactccaagtgcatcattcagatgtctggaagcaccaccaccattgttaaccccaaacagcccaaggagacgcccaaaagcttcagctttgactactcctactggtcgcacacctcacctgaggacatcaactacgcgtcgcagaagcaggtgtaccgggacatcggcgaggagatgctgcagcatgcctttgagggatacaacgtgtgcatcttcgcctatgggcagacgggtgccggcaagtcctacaccatgatgggcaagcaggagaaggaccagcagggcatcatcccacagctctgcgaggacctcttctctcggatcaacgacacgaccaacgacaacatgtcctactccgtggaggtcagctacatggagatttactgtgagcgcgtccgtgacctcctgaaccccaagaacaagggcaaccttcgcgtgagggagcacccactgctggggccctacgtggaggacctctccaagctggctgtcacctcctacaatgacatccaggacctcatggactcagggaacaaggccaggaccgtggcggccaccaacatgaatgagaccagcagtcgctcccacgccgtcttcaacatcatcttcacccagaagcgccatgacgcagagaccaatatcaccacggagaaggtgagcaaaatcagcctggtggacctggctgggagcgagcgggctgactccacgggagccaagggcacgcgcctcaaggagggggccaacatcaacaagtcgctgaccaccctgggcaaggtcatctccgccctggctgaaatggactccggacccaacaagaacaagaaaaagaagaagacagatttcattccgtaccgagattccgtgttgacctggctcctccgggaaaacctgggcggtaactcaaggacagctatggtggcagccttgagtcctgcagacatcaactacgatgagacccttagcacgctgaggtatgctgaccgggccaagcagatccgctgcaatgctgtcatcaatgaggaccccaacaacaagctgatccgcgagctgaaggatgaggtgacccggctgcgggaccttctgtacgcccagggtcttggcgacatcactgacatgaccaatgccctggtgggtatgagcccctcatcctcgctctcagccctgtccagccgcgcggcctccgtgtccagcctccacgagcgcatcttgtttgccccgggcagcgaggaggccattgaaagactgaaggaaacagagaagatcatagctgagctcaatgagacctgggaggagaagctgcggcggacagaagccatccggatggagagggaagccctgctggccgagatgggtgtggccatgagggaggatggcggcaccttgggcgtattctctcccaaaaagacaccacacctcgtcaacctgaacgaggacccgctgatgtctgagtgcctgctctactacatcaaggatgggatcaccagagtgggcagggaggatggcgagaggcggcaggacattgttctgagtgggcacttcatcaaggaggagcactgcgtcttccggagcgactccaggggaggcagcgaagctgtggtgaccttggagccctgtgagggggcagacacctacgtcaatggcaagaaagtcacagagcccagcatcctgcgttcaggaaaccgcatcatcatgggtaagagccatgtgttccggttcaaccaccccgagcaggcccggcaggagcgtgagcgcacgccttgtgcggagacgccagctgagcctgtggactgggccttcgcccagcgtgagctgctggagaagcagggcatcgacatgaagcaggagatggagcagaggctccaggaactggaggaccagtaccgccgcgagcgggaggaggccacctacctgctggagcagcagcggctggactatgagagcaagctggaggctctgcagaagcagatggactccaggtactacccggaggtgaacgaggaggaggaggagcccgaggatgaagtccagtggacagagcgggagtgtgagctggcgctctgggccttccggaagtggaagtggtaccagttcacgtctctgcgggacctgctgtggggcaacgccatcttcctcaaggaggccaatgccatcagcgtggagctgaaaaagaaggtacaattccagtttgtcctcctgacggacacactctactcccctctgccacccgacctgctgcccccagaggccgccaaagaccgagagacgcggcccttcccccgcaccattgtggccgtggaggtccaggaccagaagaacggggccacccactactggacgctggagaagctcaggcagcgtctggacctgatgcgggagatgtacgaccgcgctgcagaggtgccctccagtgtcatcgaggactgtgacaacgtggtgaccggcggagaccccttctatgaccgcttcccctggttccggctggtgggcagggccttcgtgtacctgagcaacctgctgtaccccgttcccctggtacaccgtgtggcaatcgtcagcgagaagggcgaggtgaagggcttcctccgcgtggccgtccaggccatctcagccgatgaagaggcccctgattatggctctggcgtccgccagtcgggaactgctaaaatctcctttgatgaccagcattttgaaaagttccagtccgagtcttgccccgtggtggggatgtcccgctcgggaacctcccaggaagagcttcgcatcgtggagggccagggccagggtgcagacgtggggccctcagccgatgaagtcaacaacaacacctgttcagcagtgcccccagaaggcctcctcctagacagctctgagaaagccgccctggatgggcccctggatgctgccctggaccacctccgcctgggcaacaccttcaccttccgtgtgacagtcctgcaggcgtccagcatctctgccgaatatgccgacatcttctgccagttcaacttcatccaccgccacgacgaggccttctccacagagcccctgaagaacacaggcagaggccccccacttggcttctaccacgtccagaacatcgcagtggaggtgaccaagtccttcattgagtacatcaagagccagcccattgttttcgaggtctttggccactaccagcagcacccgttcccgcccctctgcaaggacgtgctcagccccctgaggccctcgcgccgccacttccctcgggtcatgccactgtccaagccagtgcccgccaccaagctcagcacactgacgcggccctgtccgggaccctgccactgcaagtacgacctgctggtctacttcgagatctgtgagctggaggccaacggcgattacatcccggccgtggtggaccaccgtgggggcatgccatgcatggggaccttcctcctccaccagggcatccagcgacggattacggtgacactactgcatgagacaggcagccatatccgctggaaggaagtgcgcgagctggtcgtgggccgcatccgaaacactccagagaccgacgagtccctgatcgaccccaacatcttgtctctcaacatcctctcttccggatacatccacccagcccaagatgaccggaccttttaccaatttgaggctgcgtgggacagctccatgcacaactctctcctgctgaaccgggtcaccccttatcgagagaaaatctacatgacactctccgcttatatcgagatggagaactgcacccagccggctgttgtcaccaaggacttctgcatggtcttctattcccgtgatgccaagctgccagcctcgcgctccatccgcaacctctttggcagtgggagccttcgggcctcagagagtaaccgtgtgactggtgtgtacgagctcagcctgtgccacgtggctgacgcgggcagcccagggatgcagcgccggcgccgacgagtcctggacacatctgtggcctatgtccggggcgaggagaacctggcaggctggaggccccggagtgacagtctcattctggaccaccagtgggagctggagaagctgagcctcctgcaggaggtggagaagactaggcactacctgctcctgcgggagaagctggagaccgcccagcggcctgtcccggaggcactgtccccggccttcagcgaggactctgagtcccatggctcctccagcgcctcctccccgctctcggctgagggccgcccatcacccctggaggctcccaacgagaggcagcgggagctggccgtcaagtgcttgcgcctgctcacgcacacattcaacagagagtacacacacagccacgtctgcgtcagtgccagcgagagcaagctctccgagatgtctgtcaccctgctccgggacccgtcgatgtcccctctaggggtggccactctcaccccctcctccacttgcccctctctggttgaagggcggtacggtgccactgacctgaggaccccgcagccctgctcccggccagccagcccagagcccgagctgctgccagaggccgactccaagaagctcccttcccctgcccgggcaacagagacagacaaggagccccagcgcctgctggtccctgacatccaggagatccgagtcagcccgatcgtttccaagaaggggtacctgcacttcctggagccgcacacgtcaggctgggccaggcgcttcgtggtggtgcggcgcccctatgcctacatgtacaacagcgacaaggacaccgtggagcggttcgtgctcaacctggccactgcccaggtggagtacagtgaggaccagcaggctatgctcaagacacccaacacattcgcggtgtgcacggaacaccgcggcatcctgctgcaggccgccagcgacaaggacatgcatgactggctgtacgccttcaaccccctcctggccgggaccatacggtccaagctctccagaaggaggtctgcccagatgcgggtctgaacctgagccctcccgtgacagccggcaggcccagcccatcccctccctcatcctcgtctgtcctgtcacctgccgcccagcccctctcctgccagacagcccacgaccgggtcgaccccccaggggacgcccatgccaggcccggggacctgtgccacacgaccagctgtgctcccagcagaggctgtgcgtgtcagttcttcttgcagaatgtgctctggtggaacaagttgggagaggctgggggggccaagggcacaggttacgggggttcttgctgccgttctaatatttttttaagcatagacagacttataattaatatacgttagttagtgacattgaaacagtcaactcggaaattaactataagacttgttctatttataagtatttatttctaatgcctccacatagccctgtaatattcagatggaacccccaaccacctccaccctgtttgttcccacatgtgtctcccaagcctgctagggacaggcagggcagggacagccaccttggaaggccgcagtgaggagctgtctggaccagtggggcaccttggggctagcacacgggtgtatcgcctgggccccaggcttctccatggccacatgggtcctgggtgtatgtgtgggagagtgggggggtgtctttggtgcctgaagtctgcgcggcatggagggtggtgtgagttcctctggtgggagggagaacgcacatctcttctgggcggccacctgaggagtgactccaagaagagttccggcagctttccccaggaaagggtgaggggtgacactcggctctggctctgagatgaggcagacggcacccaggctgtgatctgtcctgggcggggaccaggagggagcggggtcgggatcacctgccagtgtgcagactctgggactgcgtgctgtctccggaccatcagggtagggtggtgggttgagaccaggaagtcagggaagatcggaattcagggcgacggtctaggtgtcgagggctgtggcgcagcctcttcagctgcggcgagaaatggagtgagtcaaggtagcttctgggaagaaatgctgccattagcaggtttcttgcaaagactttcctctctttgttcccagggcagagagtttctgtgagtcccactgagaaaatcccatggggtgggggtatcctggtcggtcggcaatggagggtggctggcttggtggttattgtcttcaaggagctcttcgctgctgcatctgcggtgtccctttgttcttgtcccatttcaccccctctgcagacaccaatgtccgagggccacccaggacaggacgggggtcagccccaagctgagagtctggtcataggagtcatgtccagaggcctagggaggttttagggccctccccacccacacccacaggtcgatttggtctctttttagctcaaggaaagacagtagccaagcaacagagcccctctcccgccgtggcccgtgggagcagttacatcgggtctggtgctccagacctagggcccagcactttcatcagatcctgcctcctggagtgggggaaacgcagcaccccactggttctgaggcccctaccctcccaggctgtcccacgtgatgctgacatgagcctcagagaccccaatcccatgcctgggggagagacagcggctcaggagtgggggagcacgggccttctacaccacatgggaaggctctggcatgaggttttcctttgggaaggttgtttgggcccctgaagttccatctccgagagtggtgtgcagggcaggccagggcccatgctggctgcagtctctgtggctgcctgcctgggccagcctgtttgggagctgggactgtgggctcgccttttcgtacctgggctcaggtgcggtgtggccaccgccaccctcatcccctgcctgggaggctctcccaggggctgatgggggggttctgtgagggagaatcagggctcgggaagccacgcctgggaaggcaggacacaggacacagcagctttctttggaaatctcccaggtgaggattcacatcccaaaataaaattcagaagccaggtggcctgtgctccccatgggtgacctctggaggcagtggaccaagatgcagcaaggagaggatgcagaacagcttcttgcagaagcacctgctccggcatccagcgctgcctggaggcaggaaggagaggcagggcaggacacgctggtctgagatgagggggagccccacgggccccaggcaggctagaggaggcacaggccctgccacggccaactcaggtcagccagcctgaggctgtggcctccaaagggtctgggcgcaccccccaggtcgcaggtgtctgaggccagccaacctgcagagcactcgcggcgtgggtgggctgagtggaggtgcctgggagctgcctaaattcagaagcctccactgccatggagactgcctggctgtgtcctctcagccaggtgccgtgttggccttggctaggacccagagccacactgcagcctccgccgactccctccttccgggcctgctctagtgaggagtgcctaagccaggacccagaaactcagagttgatggtgagaggaggccgcctgagtcaggacacctggcttcttgaaaagttcgctctgccagaaaccacctagggaccacgttagctgccttccttgagctccccaggagtcggtttccacatctgtgaggtggagggctggtgtggaggtgctcatggggtgcggtgcttgggagacagccaggcccagggtggctgctgcttcctgctaagtgggggaggtgagacagatctggaaagccgtctccctcagatggtttcatttaatgctttatactgccgagtctgggggcttgttttggtttgggggcagccatcctccaccagaaagggggagctccttccgctgcggctccagatagatggggatgccgggctccaggccgaccagcacttgggatctgatgggacacggccagtgcctaggggtgccaagtccaaggcctcccactgggagtcatcgctgagaagatgccaatgtttcatccaccggctgcacaggcacaaactcccccacccaggacggctgtgatgaggtggccctccctgtcaaccctggtccctggagtccccagcacctggggccctggtggggctgatgtcacaggtgtttactgtgctgctgcactggtcctatgccagcctcacccatgtggggaccacggaaggcacactcccttacccccggtgccgggccgtgcggtcccccagacggacagcagctgtggcgacctgcgtttctccctgggcctgtgcttcctgtagttagtccgtcccctggttcccctgtggctcagaggccgcgtccctggcttgtacatatgtgattgctgtgggcacaccccagaccccatgtcatagctgccgtcccgacgtcacgacgcctgtcccgatgtcttacacccgagtgttagccctaggctcctgtactgtgcgtgcacttgaggctctgtccaattaagaaataaatgtggctcttactcaacacaaaaaaaaaaaaaaaaaaaaaaaaaaaa

SEQ ID NO: 32 - protein, 1690 aa, Homo sapiens
MAGASVKVAVRVRPFNSREMSRDSKCIIQMSGSTTTIVNPKQPKETPKSFSFDYSYWSHTSPEDINYASQKQVYRDIGEEMLQHAFEGYNVCIFAYGQTGAGKSYTMMGKQEKDQQGIIPQLCEDLFSRINDTTNDNMSYSVEVSYMEIYCERVRDLLNPKNKGNLRVREHPLLGPYVEDLSKLAVTSYNDIQDLMDSGNKARTVAATNMNETSSRSHAVFNIIFTQKRHDAETNITTEKVSKISLVDLAGSERADSTGAKGTRLKEGANINKSLTTLGKVISALAEMDSGPNKNKKKKKTDFIPYRDSVLTWLLRENLGGNSRTAMVAALSPADINYDETLSTLRYADRAKQIRCNAVINEDPNNKLIRELKDEVTRLRDLLYAQGLGDITDMTNALVGMSPSSSLSALSSRAASVSSLHERILFAPGSEEAIERLKETEKIIAELNETWEEKLRRTEAIRMEREALLAEMGVAMREDGGTLGVFSPKKTPHLVNLNEDPLMSECLLYYIKDGITRVGREDGERRQDIVLSGHFIKEEHCVFRSDSRGGSEAVVTLEPCEGADTYVNGKKVTEPSILRSGNRIIMGKSHVFRFNHPEQARQERERTPCAETPAEPVDWAFAQRELLEKQGIDMKQEMEQRLQELEDQYRREREEATYLLEQQRLDYESKLEALQKQMDSRYYPEVNEEEEEPEDEVQWTERECELALWAFRKWKWYQFTSLRDLLWGNAIFLKEANAISVELKKKVQFQFVLLTDTLYSPLPPDLLPPEAAKDRETRPFPRTIVAVEVQDQKNGATHYWTLEKLRQRLDLMREMYDRAAEVPSSVIEDCDNVVTGGDPFYDRFPWFRLVGRAFVYLSNLLYPVPLVHRVAIVSEKGEVKGFLRVAVQAISADEEAPDYGSGVRQSGTAKISFDDQHFEKFQSESCPVVGMSRSGTSQEELRIVEGQGQGADVGPSADEVNNNTCSAVPPEGLLLDSSEKAALDGPLDAALDHLRLGNTFTFRVTVLQASSISAEYADIFCQFNFIHRHDEAFSTEPLKNTGRGPPLGFYHVQNIAVEVTKSFIEYIKSQPIVFEVFGHYQQHPFPPLCKDVLSPLRPSRRHFPRVMPLSKPVPATKLSTLTRPCPGPCHCKYDLLVYFEICELEANGDYIPAVVDHRGGMPCMGTFLLHQGIQRRITVTLLHETGSHIRWKEVRELVVGRIRNTPETDESLIDPNILSLNILSSGYIHPAQDDRTFYQFEAAWDSSMHNSLLLNRVTPYREKIYMTLSAYIEMENCTQPAVVTKDFCMVFYSRDAKLPASRSIRNLFGSGSLRASESNRVTGVYELSLCHVADAGSPGMQRRRRRVLDTSVAYVRGEENLAGWRPRSDSLILDHQWELEKLSLLQEVEKTRHYLLLREKLETAQRPVPEALSPAFSEDSESHGSSSASSPLSAEGRPSPLEAPNERQRELAVKCLRLLTHTFNREYTHSHVCVSASESKLSEMSVTLLRDPSMSPLGVATLTPSSTCPSLVEGRYGATDLRTPQPCSRPASPEPELLPEADSKKLPSPARATETDKEPQRLLVPDIQEIRVSPIVSKKGYLHFLEPHTSGWARRFVVVRRPYAYMYNSDKDTVERFVLNLATAQVEYSEDQQAMLKTPNTFAVCTEHRGILLQAASDKDMHDWLYAFNPLLAGTIRSKLSRRRSAQMRV

SEQ ID NO: 33 - protein, 446 aa, Homo sapiens
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 34 - DNA, 2776 bp, Homo sapiens
ggctcgctgcctcgcattgccacaggctcctgagaggtcgcgggcagtgcctgcggggaggcgcggggccctgctctgtagggctgaaggccgcccgaggttcgccaaggctctgggctctcgaaaggaagccaagaaaagaagctgcccaggtgaccagtcctgggagtgctctctcccaaggaagctccgagcgcccaggagcccttagccggggtctagtgccctttgaacaatctccagctcttcaaggaagtgggctgccgccgcctctcttgggacctggcctgggatcctttccccaaacgcaccccggcgatttttgcgcaccgggagccgaacccctgctgcgcgcagctggctgggctcaggcgcgcttcctcaacgtttcggagccgctgcccccagcgaagtccacattccaagctccaggggctttgagagagacgaccccaaggcaaggcgtttggagagctgctgaggagccaggggcttggaggagcgagaagacatgtattttcagctgagtctcagaaggggagaatctcctgtcaccaccagaaaagcaacagccccgaaatgtgattgcaactgactagcagagcagaggcccaggagtcactggattgatgatttagaatatgctaaaaagccagtgctttatttggggaattcaggggctttctggtgcccaagacagtgacctgcagatgaggactctgaacacctctgccatggacgggactgggctggtggtggagagggacttctctgttcgtatcctcactgcctgtttcctatcgctgctcatcctgtccacgctcctggggaacacgctggtctgtgctgccgttatcaggttccgacacctgcggtccaaggtgaccaacttctttgtcatctccttggctgtgtcagatctcttggtggcagtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaataagcttttacatccctgtggccatcatgattgtcacctacaccaggatctacaggattgctcagaaacaaatacggcgcattgcggccttggagagggcagcagtccacgccaagaattgccagaccaccacaggtaatggaaagcctgtcgaatgttctcaaccggaaagttcttttaagatgtccttcaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaacctgaactcgcagatgaatcctgccacacatgctcatcccaaaagctagaggagattgctctggggtttgctattaagaaactaaggtacggtgagactctgaggtgtcaggagagccctctgctgctttccaacacacaattaactccgtttccaaatacattccagtgtattttctgtgttgttcatagtcaatcaaacagggacactacaaacatggggagccataagggacatgtctttggcttcagaattgtttttagaaatttattcttatcttaggatttaccaaatagggcaaagaatcaacagtgaacagcttcacttaaaatcaaatttttctgggaagaaaatgagatgggttgagtttgctgtatacaaacaggtgctaacactgttcccagcaaagttttcagattgtaaaggtaggtgcatgccttcataaattatttctaaaacattaattgaggcttacagtaggagtgagaaatttttttccagaattgagagatgttttgttgatattggttctatttatttattgtatatatggatatttttaatttatgatataataaatatatatttatcatatttaataggataaattaatgagttttatccaagaccttacaaccacatttctggccatttaactagcactttataagccaatgaagcaaacacacagactctgtgagattctaaatgttcatgtgtaacttctaga

SEQ ID NO: 35 - DNA, 2643 bp, Homo sapiens
ggcagccgtccggggccgccactctcctcggccggtccctggctcccggaggcggccgcgcgtggatgcggcgggagctggaagcctcaagcagccggcgccgtctctgccccggggcgccctatggcttgaagagcctggccacccagtggctccaccgccctgatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacccggacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctgactctgctgcctgcccgcacagcagcctgcttcccacctccctgcccaggccggccagcctcacccttgcgaaccgtgagcaggaaggcctgggtggatcggcctcctcttcaccccggcaggccctgcagtgttcgcttggctccatgctcctcactgcccgcacaccctcactctgccagggcagtgctagtgagctgggcatggtaccagccctggggctgggccccccagctcaggggcagctcatagagtcccccctcccacctccagtccccctatccttggcaccaaagatgcagccgccttccttgaccttcctctggggctctagggttgctggagcctgagtcagggcccagaggctgagttttctctttgtggggcttggcgtggagcaggcggtggggagagatggacagttcacaccctgcaaggcccacaggaggcaagcaagctctcttgccgaggagccaggcaacttcagtcctgggagacccatgtaaataccagactgcaggttggaccccagagattcccaagccaaaaaccttagctccctcccgcaccccgatgtggacctctactttccaggctagtccggacccacctcaccccgttacagctccccaagtggtttccacatgctctgagaagaggagccctcatcttgaagggcccaggagggtctatggggagaggaactccttggcctagcccaccctgctgccttctgacggccctgcaatgtatcccttctcacagcacatgctggccagcctggggcctggcagggaggtcaggccctggaactctatctgggcctgggctaggggacatcagaggttctttgagggactgcctctgccacactctgacgcaaaaccactttccttttctattccttctggcctttcctctctcctgtttcccttcccttccactgcctctgccttagaggagcccacggctaagaggctgctgaaaaccatctggcctggcctggccctgccctgaggaaggaggggaagctgcagcttgggagagcccctggggcctagactctgtaacatcactatccatgcaccaaactaataaaactttgacgagtcaccttccaggacccctgggtaaaaaaaaaaaaaaa

SEQ ID NO: 36 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 37 - protein, 399 aa, Homo sapiens [sequence omitted]

SEQ ID NO: 38 - DNA, 1727 bp, Homo sapiens
  misc_feature (525)..(525): n is a, c, g, or t
atggagctcaaaggaaggaattaaaattttatctgttttgttcattgctctatctccaactctcacaacagtgcctactatatagaaaatgctcaataaatatttgctgatgcaataaataaaaaaaatgtaactaagcaaccaagccccaaagagtctgattttattaatattgttttctgtctcctcacaggaagccccttggcatcacgcacctcctctgggctatggcatctctgagtcagctgagtagccacctgaactacacctgtggggcagagaactccacaggtgccagccaggcccgcccacatgcctactatgccctctcctactgcgcgctcatcctggccatcgtcttcggcaatggcctgctgtgcatggctgtgctgaaggagcgggccctgcagactaccaccaactacttagtagtgagcctggctgtggcagacttgctggtggccaccttggtgatgccctgggtggtatacctggaggtgagtagacttcaggtgcatgttgacnctaacatccttgtctctgatacccaggtgacaggtggagtctggaatttcagcatttgctgtgatgtttttgtcaccctggatgtcatgatgtgtacagccagcatccttaatctctgtgccatcagcatagacaggtagggctgcaattcccttnagcctatccctctccccttgctttttcaaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggtaacagtgatggcttcatttcntttaccttcccccttctcatccacaggggaccccactgtctgctccatctccaaccctgattttgtcatctactcttcagtggtgtccttctacctgccctttggagtgactgtccttgtctatgccagaatctatgtggtgctgaaacaaaggagacggaaaaggatcctcactcgacagaacagtcagtgcaacagtgtcaggcctggcttcccccaacaaaccctctctcctgacccggcacatctggagctgaagcgttactacagcatctgccaggacactgccttgggtggaccaggcttccaagaaagaggaggagagttgaaaagagaggagaagactcggaattccctgagtcccaccatagcgcccaagctcagcttagaagttcgaaaactcagcaatggcagattatcgacatctttgaagctggggcccctgcaacctcggggagtgccacttcgggagaagaaggcaacccaaatggtggccattgtgcttggtaagtttgggttggcttgagctgtgttncccagtttctctcttatttggcaacttaggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgctgagggagcagaagagggaacactctttgtacccatttctagcgccaggctgttggcccactcagaaagacagagcagtgtctccggcatgc

SEQ ID NO: 39 - protein, 419 aa, Homo sapiens
MGNRSTADADGLLAGRGPAAGASAGASAGLAGQGAAALVGGVLLIGAVLAGNSLVCVSVATERALQTPTNSFIVSLAAADLLLALLVLPLFVYSEVQGGAWLLSPRLCDALMAMDVMLCTASIFNLCAISVDRFVAVAVPLRYNRQGGSRRQLLLIGATWLLSAAVAAPVLCGLNDVRGRDPAVCRLEDRDYVVYSSVCSFFLPCPLMLLLYWATFRGLQRWEVARRAKLHGRAPRRPSGPGPPSPTPPAPRLPQDPCGPDCAPPAPGLPRGPCGPDCAPAAPSLPQDPCGPDCAPPAPGLPPDPCGSNCAPPDAVRAAALPPQTPPQTRRRRRAKITGRERKAMRVLPVVVGAFLLCWTPFFVVHITQALCPACSVPPRLVSAVTWLGYVNSALNPVIYTVFNAEFRNVFRKALRACC

SEQ ID NO: 40 - DNA, 1360 bp, Homo sapiens
atggggaaccgcagcaccgcggacgcggacgggctgctggctgggcgcgggccggccgcgggggcatctgcgggggcatctgcggggctggctgggcagggcgcggcggcgctggtggggggcgtgctgctcatcggcgcggtgctcgcggggaactcgctcgtgtgcgtgagcgtggccaccgagcgcgccctgcagacgcccaccaactccttcatcgtgagcctggcggccgccgacctcctcctcgctctcctggtgctgccgctcttcgtctactccgaggtccagggtggcgcgtggctgctgagcccccgcctgtgcgacgccctcatggccatggacgtcatgctgtgcaccgcctccatcttcaacctgtgcgccatcagcgtggacaggttcgtggccgtggccgtgccgctgcgctacaaccggcagggtgggagccgccggcagctgctgctcatcggcgccacgtggctgctgtccgcggcggtggcggcgcccgtactgtgcggcctcaacgacgtgcgcggccgcgaccccgccgtgtgccgcctggaggaccgcgactacgtggtctactcgtccgtgtgctccttcttcctaccctgcccgctcatgctgctgctctactgggccacgttccgcggcctgcagcgctgggaggtggcacgtcgcgccaagctgcacggccgcgcgccccgccgacccagcggccctggcccgccttcccccacgccacccgcgccccgcctcccccaggacccctgcggccccgactgtgcgccccccgcgcccggccttccccggggtccctgcggccccgactgtgcgcccgccgcgcccagcctcccccaggacccctgtggccccgactgtgcgccccccgcgcccggcctccccccggacccctgcggctccaactgtgctccccccgacgccgtcagagccgccgcgctcccaccccagactccaccgcagacccgcaggaggcggcgtgccaagatcaccggccgggagcgcaaggccatgagggtcctgccggtggtggtcggggccttcctgctgtgctggacgcccttcttcgtggtgcacatcacgcaggcgctgtgtcctgcctgctccgtgcccccgcggctggtcagcgccgtcacctggctgggctacgtcaacagcgccctcaaccccgtcatctacactgtcttcaacgccgagttccgcaacgtcttccgcaaggccctgcgtgcctgctgctgagccgggcacccccggacgccccccggcctgatggccaggcctcagggaccaaggagatggggagggcgcttttgtacgttaattaaacaaattccttccc

SEQ ID NO: 41 - protein, 477 aa, Homo sapiens
MLPPGSNGTAYPGQFALYQQLAQGNAVGGSAGAPPLGPSQVVTACLLTLLIIWTLLGNVLVCAAIVRSRHLRANMTNVFIVSLAVSDLFVALLVMPWKAVAEVAGYWPFGAFCDVWVAFDIMCSTASILNLCVISVDRYWAISRPFRYKRKMTQRMALVMVGLAWTLSILISFIPVQLNWHRDQAASWGGLDLPNNLANWTPWEEDFWEPDVNAENCDSSLNRTYAISSSLISFYIPVAIMIVTYTRIYRIAQVQIRRISSLERAAEHAQSCRSSAACAPDTSLRASIKKETKVLKTLSVIMGVFVCCWLPFFILNCMVPFCSGHPEGPPAGFPCVSETTFDVFVWFGWANSSLNPVIYAFNADFQKVFAQLLGCSHFCSRTPVETVNISNELISYNQDIVFHKEIAAAYIHMMPNAVTPGNREVDNDEEEGPFDRMFQIYQTSPDGDPVAESVWELDCEGEISLDKITPFTPNGFH

SEQ ID NO: 42 - DNA, 2053 bp, Homo sapiens
cagggctgaagttgggaccgcgcacagaccgcccctgcagtccagcccgaaatgctgccgccaggcagcaacggcaccgcgtacccggggcagttcgctctataccagcagctggcgcaggggaacgccgtggggggctcggcgggggcaccgccactggggccctcacaggtggtcaccgcctgcctgctgaccctactcatcatctggaccctgctgggcaacgtgctggtgtgcgcagccatcgtgcggagccgccacctgcgcgccaacatgaccaacgtcttcatcgtgtctctggccgtgtcagaccttttcgtggcgctgctggtcatgccctggaaggcagtcgccgaggtggccggttactggccctttggagcgttctgcgacgtctgggtggccttcgacatcatgtgctccactgcctccatcctgaacctgtgcgtcatcagcgtggaccgctactgggccatctccaggcccttccgctacaagcgcaagatgactcagcgcatggccttggtcatggtcggcctggcatggaccttgtccatcctcatctccttcattccggtccagctcaactggcacagggaccaggcggcctcttggggcgggctggacctgccaaacaacctggccaactggacgccctgggaggaggacttttgggagcccgacgtgaatgcagagaactgtgactccagcctgaatcgaacctacgccatctcttcctcgctcatcagcttctacatccccgttgccatcatgatcgtgacctacacgcgcatctaccgcatcgcccaggtgcagatccgcaggatttcctccctggagagggccgcagagcacgcgcagagctgccggagcagcgcagcctgcgcgcccgacaccagcctgcgcgcttccatcaagaaggagaccaaggttctcaagaccctgtcggtgatcatgggggtcttcgtgtgttgctggctgcccttcttcatccttaactgcatggtccctttctgcagtggacaccctgaaggccctccggccggcttcccctgcgtcagtgagaccaccttcgacgtcttcgtctggttcggctgggctaactcctcactcaaccccgtcatctatgccttcaacgccgactttcagaaggtgtttgcccagctgctggggtgcagccacttctgctcccgcacgccggtggagacggtgaacatcagcaatgagctcatctcctacaaccaagacatcgtcttccacaaggaaatcgcagctgcctacatccacatgatgcccaacgccgttacccccggcaaccgggaggtggacaacgacgaggaggagggtcctttcgatcgcatgttccagatctatcagacgtccccagatggtgaccctgttgctgagtctgtctgggagctggactgcgagggggagatttctttagacaaaataacacctttcaccccgaatggattccattaaactgcattaagaaaccccctcatggatctgcataaccgcacagacactgacaagcacgcacacacacgcaaatacatgcctttccagtgctgctccctttatcatgtgtttctgtgtagtagctcgtgtgcttagaaacctcaccccattgattggtagttcgaagaattggcagaatcagttgcaataaactcagtcaaatgtacccagcctaccagagatggaccaacgatcctatgagagaagagagtatggtgctgggtccttaaaaaaaaaaatgatacttggtccttaaaaaatatgctctcccctccctttttaaacaaatggcttgttcagtcacttgtttgtgtttgaattgatttttaaacagcaggttgtgtgtgtgtgcagtgatgtggtgggagcacagctttcctgggtctggattcccgtggctttgtgcttatgtcatttcttctctctgtgctggtgggggcctctttaccatagcttaagaagtatccctgatttattctggtgtctaataaacacagattatttgtaaaaaaaaaaaaaaaaaaaaaaa

SEQ ID NO: 43 - protein, 121 aa, Homo sapiens
RIYVVLKQRRRKRILTRQNSQCNSVRPGFPQQTLSPDPAHLELKRYYSICQDTALGGPGFQERGGELKREEKTRNSLSPTIAPKLSLEVRKLSNGRLSTSLKLGPLQPRGVPLREKKATQM

SEQ ID NO: 44 - protein, 113 aa, Homo sapiens
NADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

(19) United States
(12) Patent Application Publication
(10) Pub. No.: US 2009/0209045 A1
(43) Pub. Date: Aug. 20, 2009

(54) SCREENING ASSAY

(75) Inventor: Jamal Nasir, Sheffield (GB)

Correspondence Address: SPECKMAN LAW GROUP PLLC, 1201 THIRD AVENUE, SUITE 330, SEATTLE, WA, 98101, US

(73) Assignee: GENOPHRENIX LIMITED, Sheffield (GB)

(21) Appl. No.: 11/577,521

(22) PCT Filed: Oct. 20, 2005
(86) PCT No.: PCT/GB05/04071

§ 371 Date: Jun. 23, 2008

(30) Foreign Application Priority Data
Oct. 21, 2004 (GB) 0423348.2

(51) Int. Cl. G01N 33/566; C07K 16/18; C12N 5/10; A01K 67/027

(52) U.S. Cl. 436/501; 530/387.3; 530/387.9; 435/325; 800/13

(57) ABSTRACT

The present invention relates to agents that modulate the interaction of a dopamine receptor interacting polypeptide (DRIP) as represented by FIGS. 1 to 11 and to screening methods for the identification of such agents.

The invention relates to a screening assay for the identification of agents which modulate the interaction of polypeptides that bind to one or more dopamine receptors.

Dopamine signalling provides a crucial mechanism for transmitting information between neurons. Release of the neurotransmitter dopamine, across the synaptic cleft and uptake by post-synaptic dopamine receptors invokes a signalling cascade, involving the activation of heterotrimeric G-proteins and second messenger pathways (Neves et al., 2002). Failure to regulate this process is associated with neuropsychiatric disorders, including Parkinson's Disease, Attention Deficit Hyperactivity Disorder (ADHD), depression (bipolar disorder) and schizophrenia (Greengard, 2001) and also addiction (e.g drug addiction such as alcohol, nicotine or cocaine addiction). Other “dopamine-mediated disorders” may include neurodegenerative disorders (e.g Alzheimer's disease, Tourette Syndrome, Parkinson's disease, Huntington's disease, Amyotrophic Lateral Sclerosis, senile chorea, Sydenham's chorea, autism, distonia, tremor, autism, head and spinal cord trauma, acute and chromic pain, epilepsy and seizures, dementia, cerebral ischemia and neuronal cell death) and disorders linked to apoptosis, particularly neuronal apoptosis.

Dopamine signalling is poorly understood at the molecular level, though the structure and function of dopamine receptors have been intensively studied (Sibley and Monsana, 1992; Missale et al. 1998). Dopamine receptors are G-protein coupled seven transmembrane receptors, which transduce extracellular signals into the cell. These receptors are classified into two groups, on the basis of their genetic and pharmacological properties. The D1-like receptors (D1, D5) lack introns and activate adenylyl cyclase causing an increase in intracellular levels of cyclic AMP (cAMP). D2-like receptors (D2, D3, D4) contain introns and inhibit adenylyl cyclase. cAMP activates protein kinases which in turn phosphohrylate target proteins, including the dopamine and cAMP regulated 32 kD phosphoprotein, DARPP-32 (Greengard, 2001). DARPP-32 is necessary to mediate the effects of dopamine, including the regulation of ion pumps, ion channels and transcription factors (for example CREB (cAMW responsive element binding protein)).

Dopamine receptors possess two important functional domains, an intracellular third loop and a C-terminal cytoplasmic domain. D1 and D5 receptors possess a short third loop with a long C-terminal. D2, D3 and D4 receptors possess a long cytoplasmic third loop and a short C-terminal region. The intracellular domains of G-protein coupled receptors form a signal transduction complex by interacting with various accessory proteins which play a critical role in signalling (Wu et al., 1998). This is a dynamic process that can involve the recruitment of cytosolic signalling proteins to the receptor, or the receptor itself can internalise in order to interact with other proteins (Lamey et al., 2002; Kabbani et al., 2004).

The ‘dopamine hypothesis’ is supported by over 30 years of molecular, clinical and pharmacological data, culminating in a Nobel Prize in 2000 to Carlsson and Greengard, in recognition of their contribution to the fields of dopamine signalling and schizophrenia (Carlsson, 2001; Greengard, 2001). For example, receptor binding studies on post-mortem brain tissue and brain imaging techniques such as Positron Emission Tomography (PET), show an increase in D4 and a decrease in D1 receptors, in regions of the brain known to be involved in schizophrenia, including the prefrontal cortex, cingulate gyri, thalamus, hippocampus and putamen (Seeman et al., 1993; Silbersweig et al., 1995; Okubo et al., 1997). However, antipsychotic drugs act by blocking dopamine receptors, primarily D2 receptors (Abi-Dargham et al., 2000; Seeman and Kapur, 2000). By contrast, drugs such as amphetamine and cocaine, which increase dopamine levels, induce psychosis (Roberts et al., 1993). These findings are further supported by studies of knock-out mice (Baik et al., 1995; Giros et al., 1996). Yet, despite the well documented role of dopamine in schizophrenia, linkage studies have failed to provide conclusive evidence for the role of dopamine receptors in schizophrenia (Coon et al., 1993).

Current drugs for treating schizophrenia, which act by blocking dopamine receptors, are either ineffective or associated with major side-effects. Zyprexa was the 5th best selling drug in the USA in 2003 with sales of $3.2 billion. However, Zyprexa leads to massive weight gain within the short-term, resulting in weight gains of up to several kg per month within the first few months. There are also long-term side effects of drugs used to treat schizophrenia. For example, antipsychotics described as typical neuroleptics, can also cause major extrapyramidal side-effects such as tremor, Parkinsonism, and tardive dyskinesia (involuntary movements, often of the tongue and face but also of the fingers, hands, legs, and trunk) after prolonged use.

There are required new targets for the development of drugs that obviate or mitigate the aforementioned disadvantages.

The present inventors have identified polypeptides that interact with dopamine receptors thereby making the identification of agents that bind to these polypeptides, either directly or indirectly, possible.

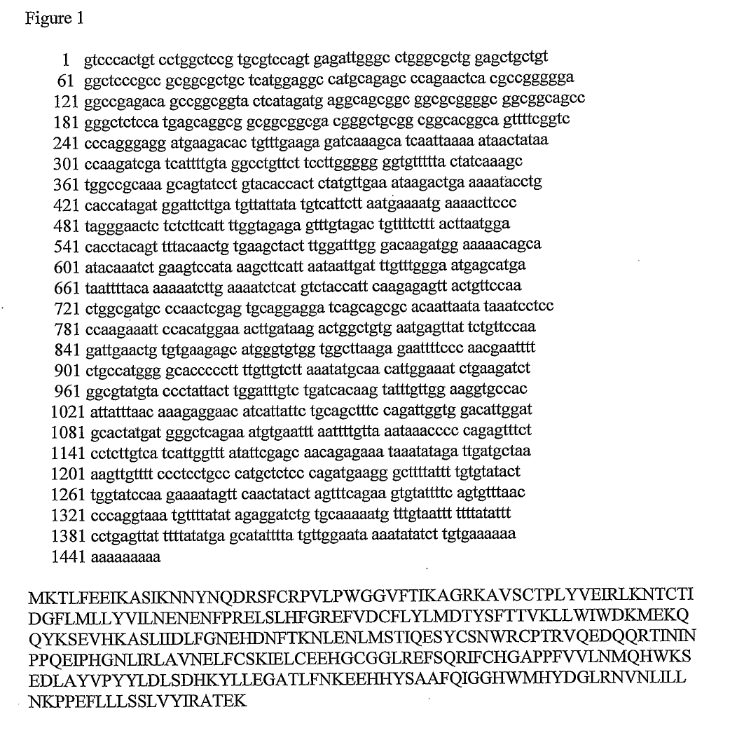

According to a first aspect of the invention there is provided a screening method for the identification of agents that modulate the interaction of a polypeptide with one or more dopamine receptors wherein said polypeptide is selected from the group consisting of:
i) a polypeptide, or fragment or variant thereof, encoded by a nucleic acid molecule consisting of a nucleic acid sequence as represented by a sequence shown in FIG. 1, 2, 3, 4, 5, 6, 7, 8, 9 10 or 11, or a sequence complementary thereto, or a fragment thereof;
ii) a polypeptide encoded by a nucleic acid molecule which hybridises to a nucleic acid molecule as defined in (i) above and which has dopamine receptor binding activity;
iii) a polypeptide comprising a nucleic acid which is degenerate as a result of the genetic code to the nucleic acid sequence defined in (i) and (ii);
   comprising the steps of
i) forming a preparation comprising said polypeptide and a dopamine receptor; and
ii) adding at least one candidate agent to be tested;
iii) determining the effect, or not, or said agent on the interation of said polypeptide with said dopamine receptor.

In a preferred method of the invention said polypeptide is represented by an amino acid sequence as shown in FIG. 1, 2, 3, 4, 5, 6, 7, 8, 9 10 or 11, or a variant polypeptide wherein said variant polypeptide sequence has been altered by addition, substitution or deletion of at least one amino acid residue. Such polypeptides, as well as the polypeptides defined in the first aspect of the invention, will be referred to herein as “dopamine receptor interacting polypeptides (DRIPs)”

“Variant(s)” of polypeptides as used herein include polypeptides that differ in amino acid sequence from a reference polypeptide. Generally, differences are limited so that the sequences of the reference and the variant are closely similar and, in many regions, identical. A variant polypeptide may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan.

The present invention further relates to variants of the above described nucleic acid sequences/molecules. In this respect, a variant may be a naturally occurring variant such as a naturally occurring allelic variant, for example, one or more single nucleotide polymorphisms (SNPs) within coding or non-coding regions of the above described nucleic acid sequences. SNPs may also occur within nucleic acid sequences neighbouring the above described nucleic acid sequences or within another nucleic acid sequence that affects the properties/functions of DRIPs.

Alternatively, the variant may be a variant that is known to occur non-naturally. Such non-naturally occurring variants of the polynucleotide may be made by mutagenesis techniques, including those applied to polynucleotides, cells or organisms.

The nucleic acid molecule of the first aspect of the invention may anneal under stringent hybridisation conditions to the nucleic acid sequence shown in FIG. 1, 2, 3, 4, 5, 6, 7, 8, 9, 10 or 11 or to its complementary strand.

Stringent hybridisation/washing conditions are well known in the art. For example, nucleic acid hybrids that are stable after washing in 0.1×SSC, 0.1% SDS at 60° C. It is well known in the art that optimal hybridisation conditions can be calculated if the sequences of the nucleic acid is known. For example, hybridisation conditions can be determined by the GC content of the nucleic acid subject to hybridisation. Please see Sambrook et al (1989) Molecular Cloning; A Laboratory Approach. A common formula for calculating the stringency conditions required to achieve hybridisation between nucleic acid molecules of a specified homology is:

Tₘ=81.5° C.+16.6 Log [Na⁺]0.41[% G+C]−0.63 (% formamide).

The nucleic acid molecule of the first aspect of the invention may comprise the sequence set out in FIG. 1, 2, 3, 4, 5, 6, 7, 8, 9 10 or 11 or a sequence which is at least 50%, 55%, 60%, 65%, 70%, 75%, 80%, 85%, 90%, 95%, for example 98%, or 99%, identical to the nucleic acid sequence set out in FIG. 1, 2, 3, 4, 5, 6, 7, 8, 9 10 or 11 respectively at the nucleic acid residue level.

“Identity”, as known in the art, is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity can be readily calculated (Computational Molecular Biology, Lesk, A. M. ed., Oxford University Press, New York, 1988; Biocomputing: Informatics and Genome Projects, Smith, D. W., ed., Academic Press, New York, 1993; Computer Analysis of Sequence Data, Part I, Griffin, A. M., AND Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or two polypeptide sequences, the term is well-known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIMA J. Applied Math., 48: 1073 (1988). Methods commonly employed to determine identity between sequences include, but are not limited to those disclosed in Carillo, H., and Lipman, D., SIAM J. Applied Math., 48: 1073 (1988). Preferred methods to determine identity are designed to give the largest match between the sequences tested. Methods to determine identity are codified in computer programs. Preferred computer program methods to determine identity between two sequences include, but are not limited to, GCG program package (Devereux, J., et al., Nucleid Acids Research 12(1): 387 (1984)), BLASTP, BLASTN, and FASTA (Atschul, S. F. et al., J. Molec. Biol. 215: 403 (1990)).

The complementary sequences of the nucleic acid sequences of the first aspect of the invention may be useful as probes or primers, or in the regulation of the gene(s) encoding the DRIPs. They may be useful in the identification and/or treatment of individuals having diseases relating to a defective or deficient DRIP, for example in the identification of polymorphisms in DRIPs. Such polymorphisms can be used as a research tool, for example in analysing DRIP function, in screening subjects for genetic risk factors or in predicting drug responses in a subject. These sequences are preferably isolated.

In a further preferred method of the invention the nucleic acid sequence comprises nucleotides 644 to 1191 of the sequence shown in FIG. 1.

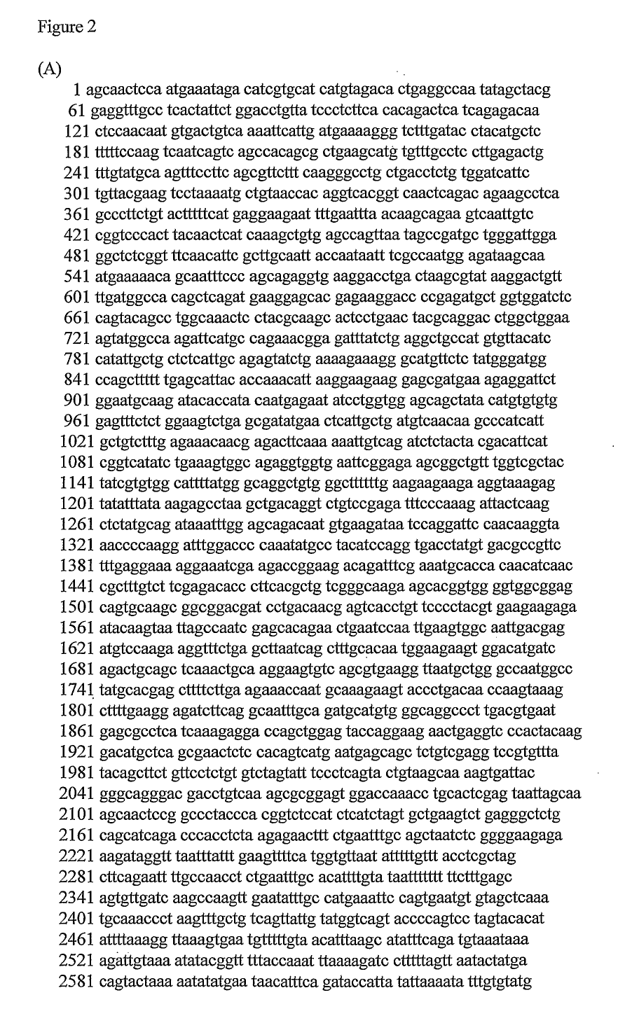

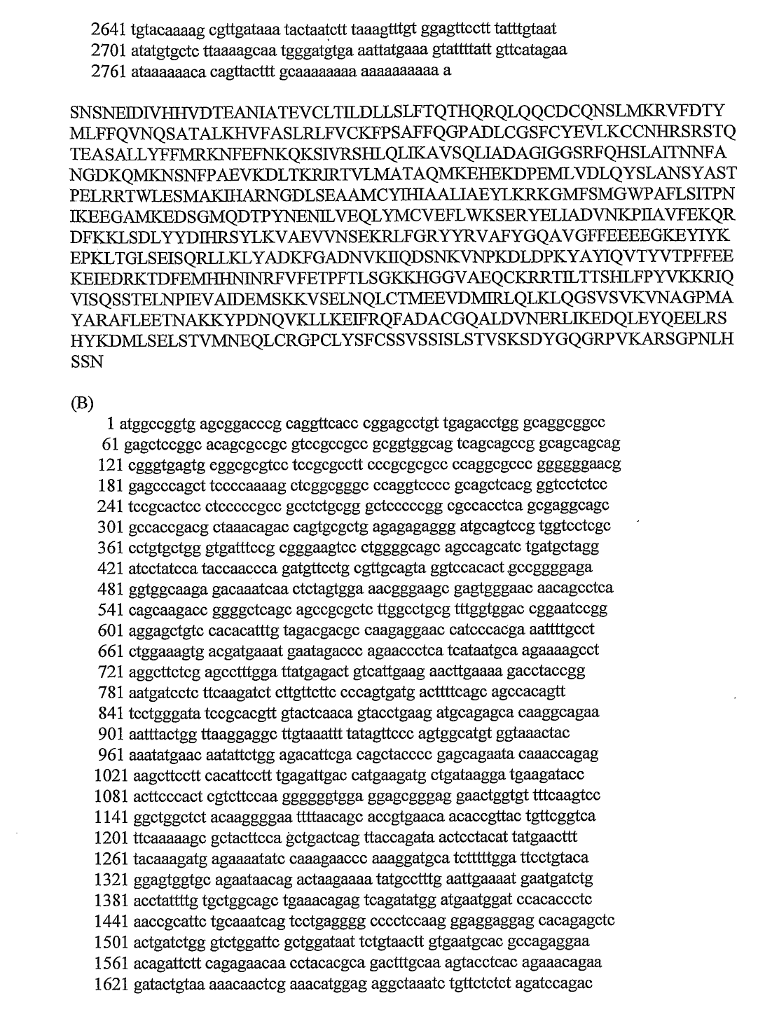

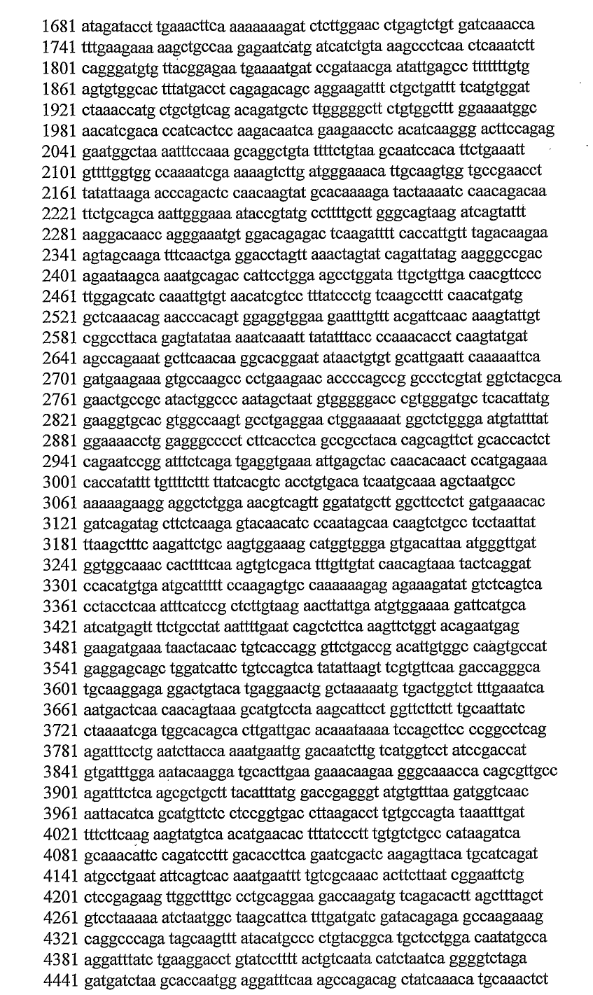

In a further preferred method of the invention the nucleic acid sequence comprises nucleotides 1 to 601 of the sequence shown in FIG. 2.

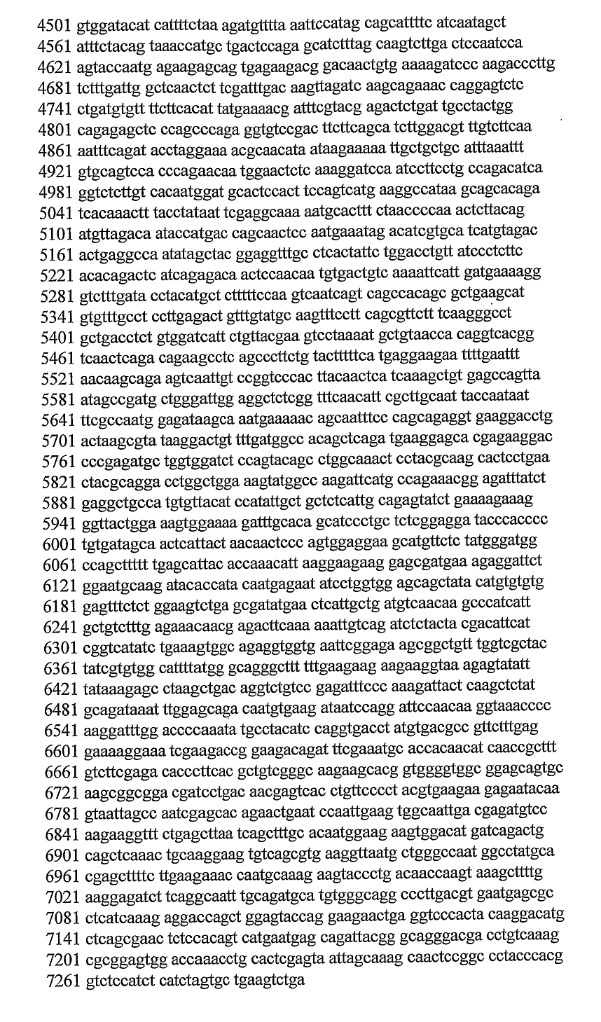

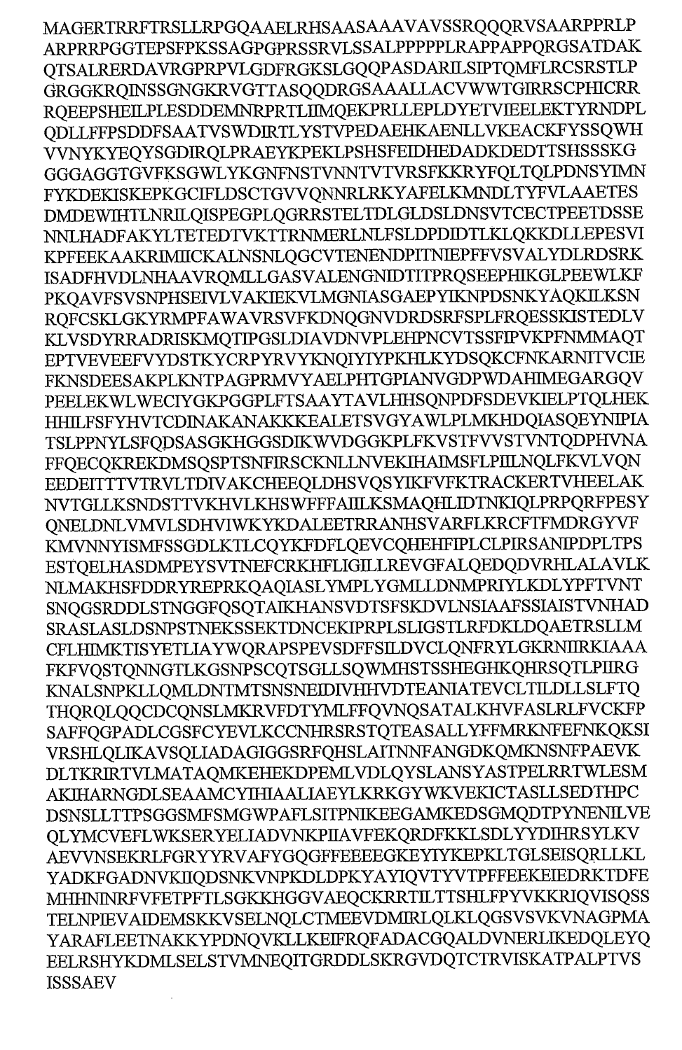

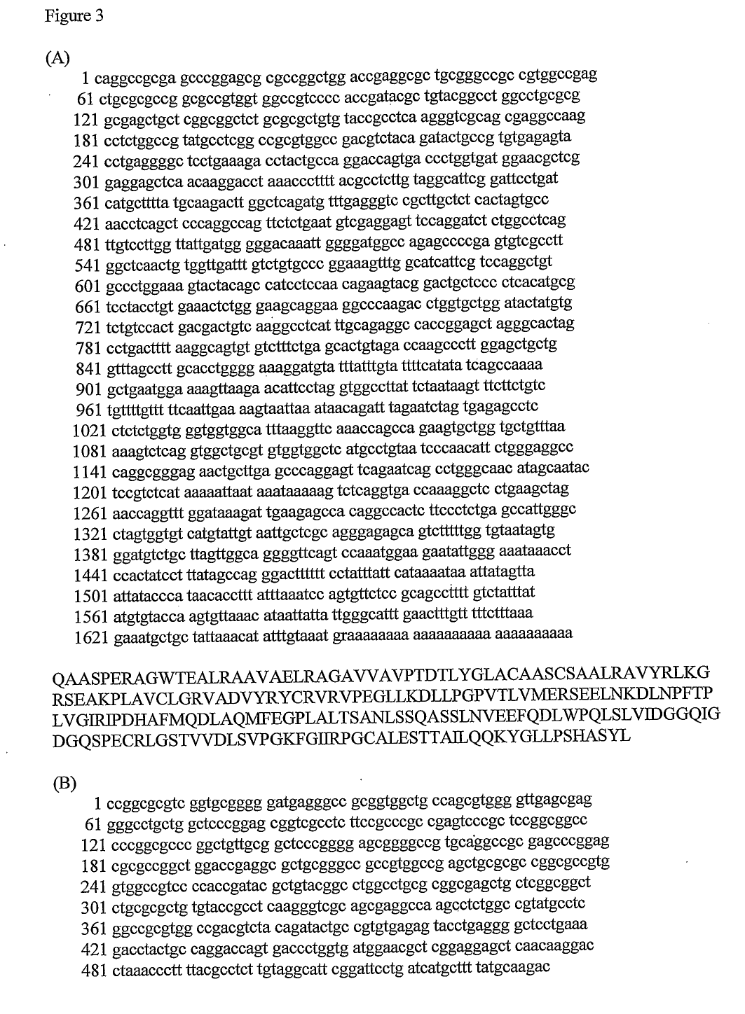

In a further preferred method of the invention the nucleic acid sequence comprises nucleotides 1 to 521 of the sequence shown in FIG. 3.

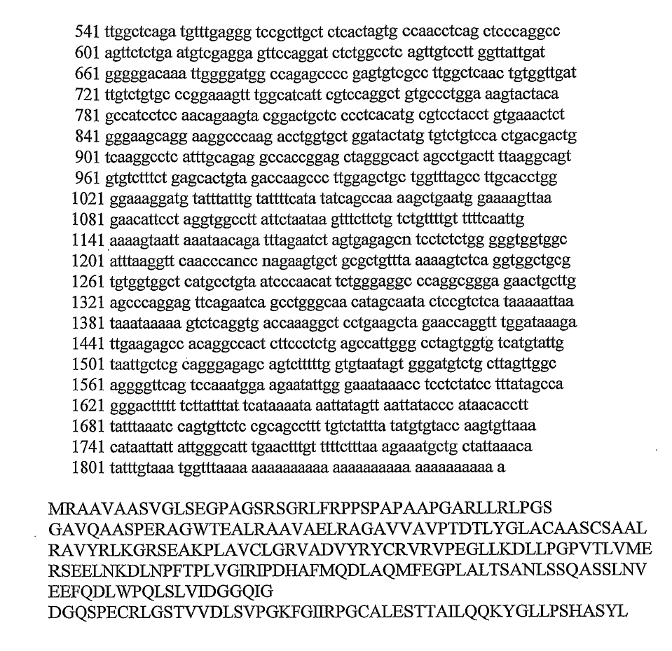

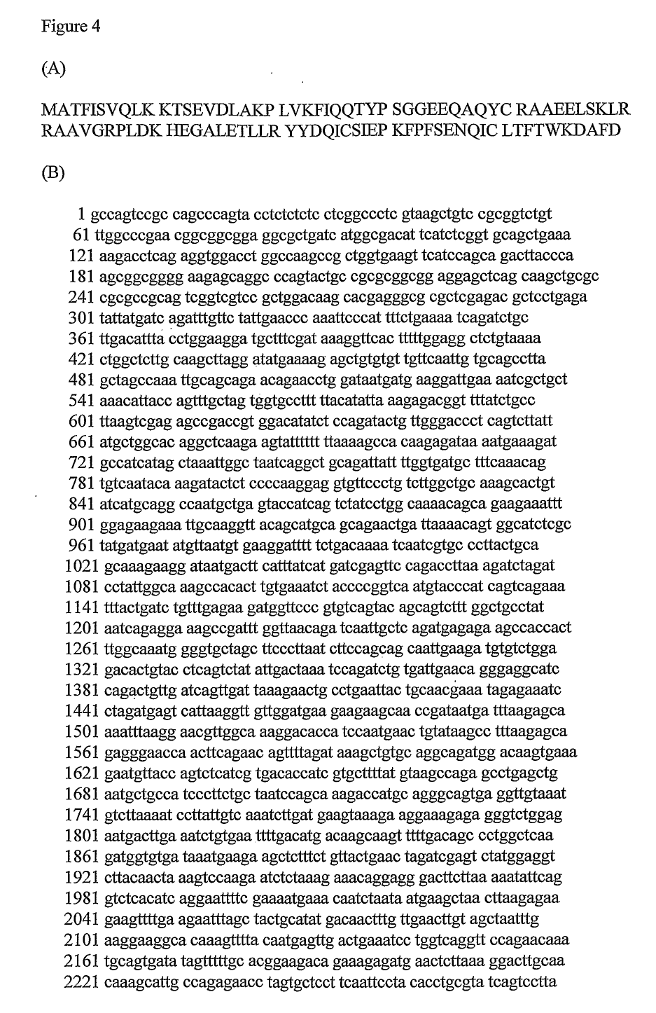

In a further preferred method of the invention the nucleic acid sequence comprises nucleotides 415 to 910 of the sequence shown in FIG. 4.

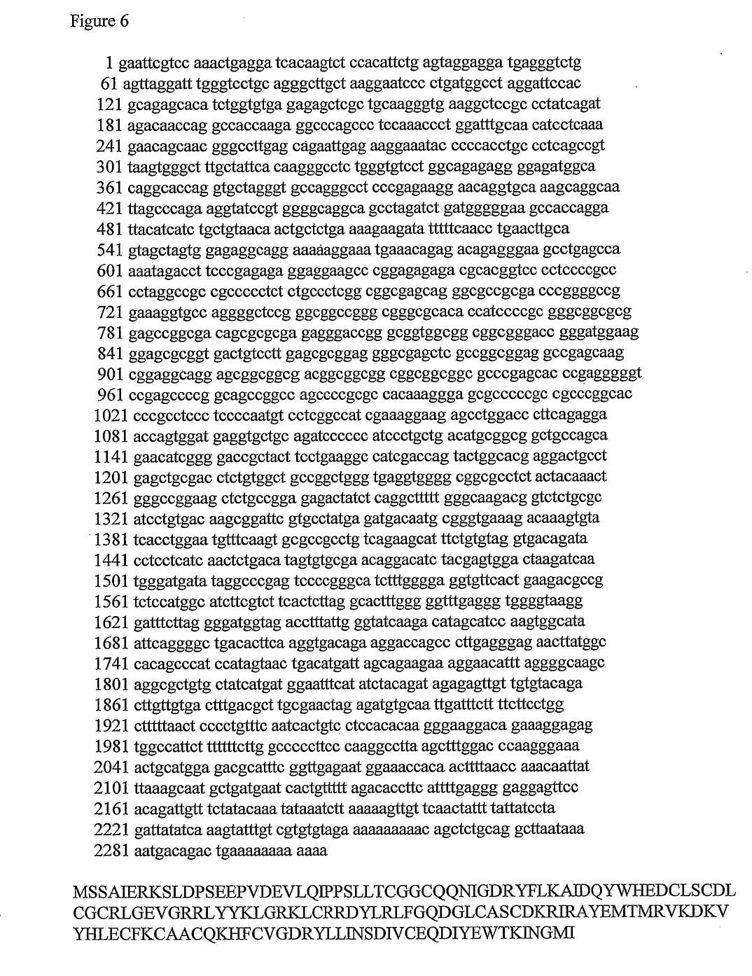

In a further preferred method of the invention the nucleic acid sequence comprises nucleotides 1058 to 1661 of the sequence shown in FIG. 6.

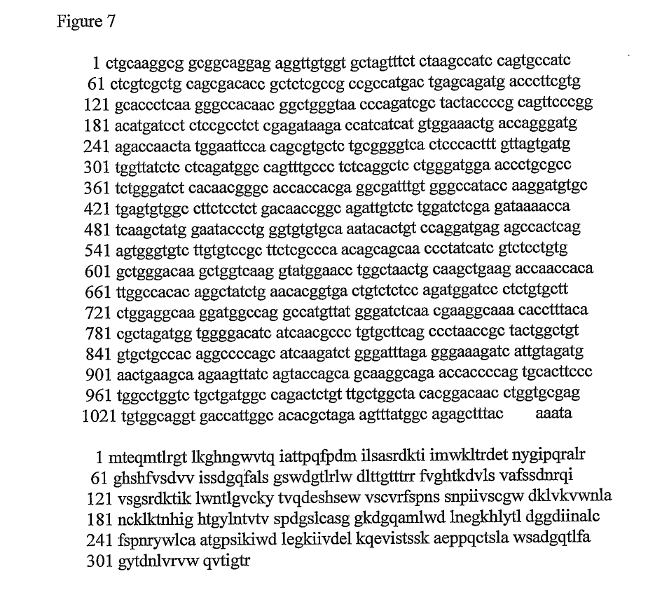

In a further preferred method of the invention the nucleic acid sequence comprises nucleotides 621 to 1069 of the sequence shown in FIG. 7.

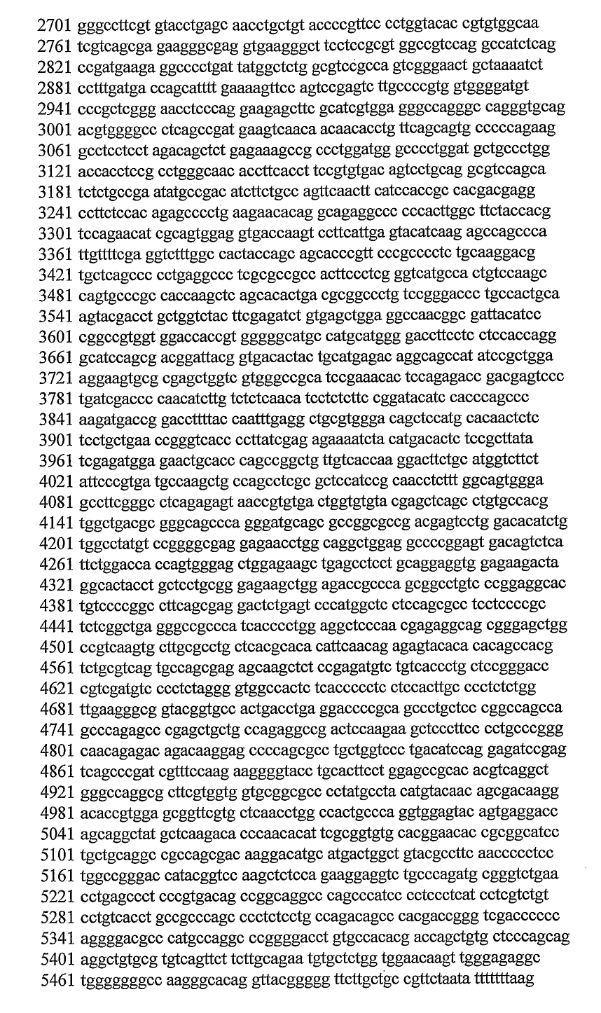

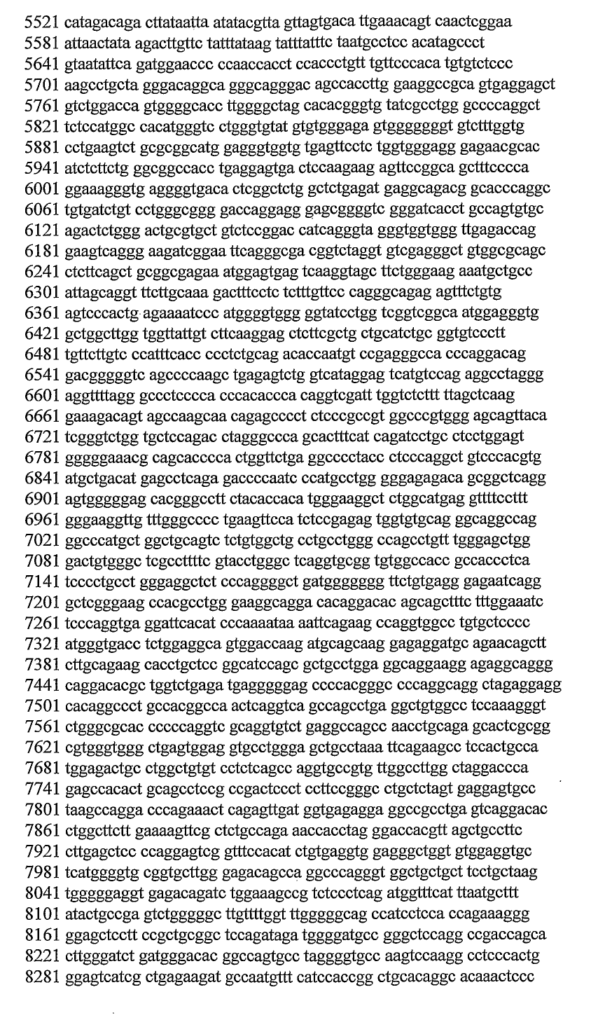

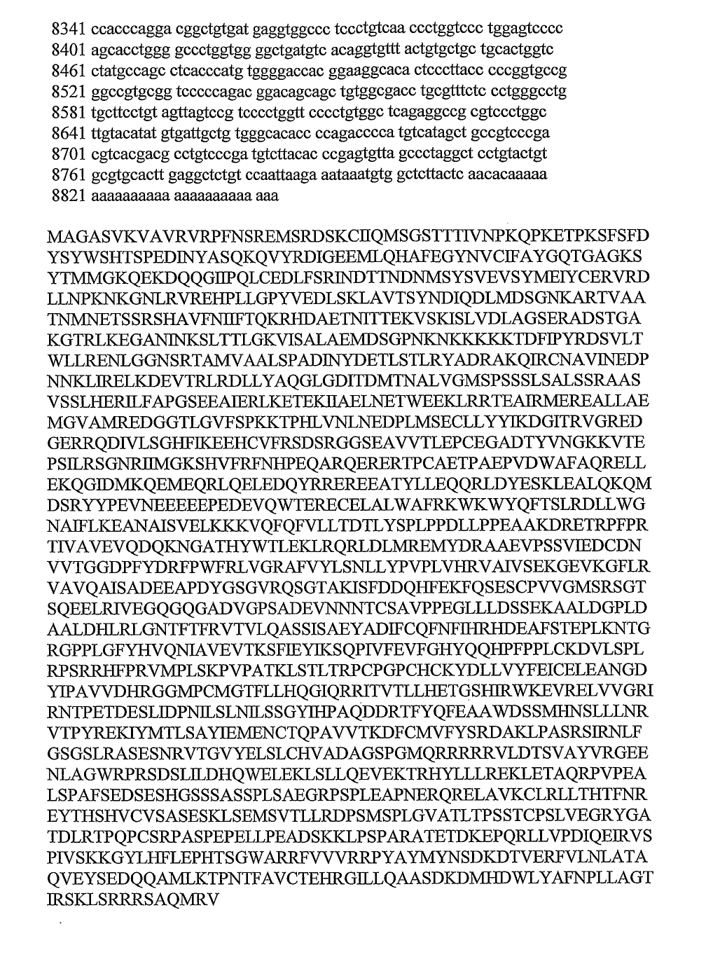

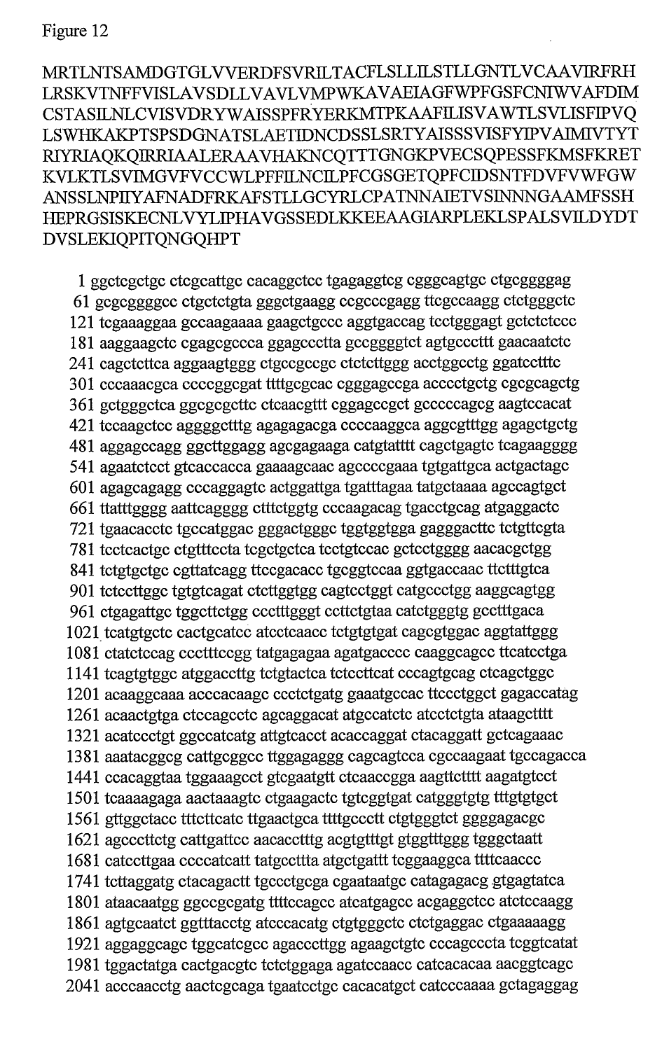

Dopamine receptors include D1-like receptors (D1, D5) and D2-like receptors (D2, D3, D4). In a preferred method of the invention the dopamine receptor is represented by a polypeptide having the amino acid sequence shown in FIG. 12, 13, 14, 15 or 16.

As used herein, the term “polypeptide” means, in general terms, a plurality of amino acid residues joined together by peptide bonds. It is used interchangeably and means the same as peptide, protein, oligopeptide, or oligomer. The term “polypeptide” is also intended to include fragments, variants including alternate splice variants or isoforms, analogues and derivatives of a polypeptide wherein the fragment, variant, analogue or derivative retains essentially the same biological activity or function as a reference protein.

As used herein “fragment” may include any contiguous 10 residue sequence, or greater, such as a 15, 20, 30, 50 or 100 residue sequence. Preferably, fragments of nucleotide or polypeptide sequences share one or more functional characteristics with DRIP or its gene.

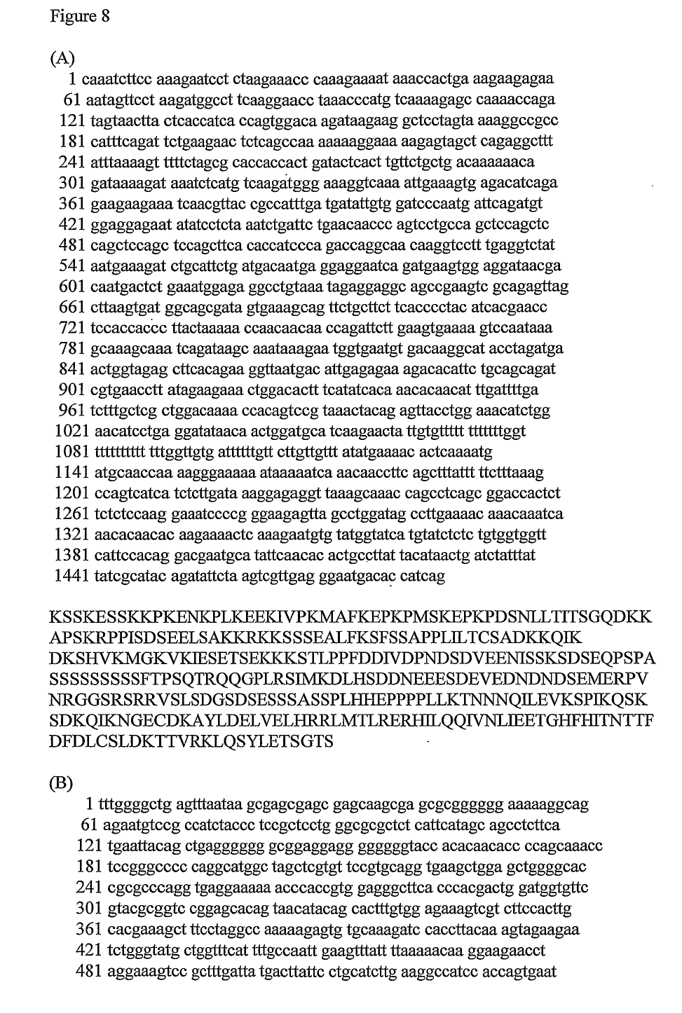

The fragments may be used in a variety of diagnostic, prognostic or therapeutic methods or may be useful as research tools for example in the screening. Fragments of the nucleic acid sequences of the first aspect of the invention, or their complements, may be used as primer sequences. For example a fragment comprising nucleotides 640 to 765 of the sequence shown in FIG. 8, which corresponds to a repeated motif encoding a polyserine tract, may be useful in diagnosis or prognosis e.g of a dopamine-mediated disorder as described herein.

In a preferred method of the invention the DRIP is expressed by a cell. In a further preferred method of the invention the dopamine receptor is expressed by a cell.

In a preferred method of the invention said cell is a cell transfected with at least one nucleic acid molecule(s) which encodes a DRIP and/or dopamine receptor. The nucleic acid molecule(s) may be provided in the form of a vector to enable the in vitro or in vivo expression of the nucleic acid molecule. Preferably, the expression of said nucleic acid molecules is regulatable. Appropriate regulatory elements, in particular promoters, will be used depending on the host cell into which the expression vector is inserted.

Preferred cells include E. coli, yeast, filamentous fungi, insect cells, mammalian cells, preferably immortalised such HEK293, CHO, HeLa, Myeloma, Jurkat or cos cell lines, monkey cell lines and derivatives thereof.

In a preferred method of the invention said cell is a brain cell, for example, a neuronal cell.

In a yet further aspect of the invention said cell is part of a transgenic animal wherein the genome of said animal has been modified to include nucleic acid molecules which encode said polypeptide and/or said dopamine receptor.

In a further aspect of the invention there is provided an agent that modulates the interaction of a polypeptide with one or more dopamine receptors wherein said polypeptide is selected from the group consisting of:
i) a polypeptide, or fragment or variant thereof, encoded by a nucleic acid molecule consisting of a nucleic acid sequence as represented by a sequence shown in FIG. 1, 2, 3, 4, 5, 6, 7, 8, 9 10 or 11, or a sequence complementary thereto, or a fragment thereof;
ii) a polypeptide encoded by a nucleic acid molecule which hybridises to a nucleic acid molecule as defined in (i) above and which has dopamine receptor binding activity;
iii) a polypeptide comprising a nucleic acid which is degenerate as a result of the genetic code to the nucleic acid sequence defined in (i) and (ii), wherein the agent is for use as a medicament.

Generally, the agent binds a dopamine receptor but is not the natural ligand of a dopamine receptor. The agent may be an agonist or antagonist of the interaction between a DRIP and one or more dopamine receptors such as shown any of FIGS. 12 to 16.

In a preferred method of the invention said agent is an antibody or an active binding fragment of an antibody, for example a monoclonal antibody. The antibody fragment may be a single chain antibody variable region fragment.

Antibodies or immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant. The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region. The variable region contains complementarity determining regions or CDR's which form an antigen binding pocket. The binding pockets comprise H and L variable regions which contribute to antigen recognition. It is possible to create single variable regions, so called single chain antibody variable region fragments (scFv's). If a hybidoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's.

Alternatively said fragments are “domain antibody fragments”. Domain antibodies are the smallest part of an antibody (approximately 13 kDa). Examples of this technology is disclosed in U.S. Pat. No. 6,248,516, U.S. Pat. No. 6,291,158 and U.S. Pat. No. 6,127,197.

In a preferred embodiment of the invention said antibody fragment is a single chain antibody variable region fragment.

In a further preferred embodiment of the invention said antibody, or binding fragment thereof, is a chimeric antibody. In an alternative preferred embodiment of the invention said antibody, or binding fragment thereof, is a humanised antibody.

A chimeric antibody is produced by recombinant methods to contain the variable region of an antibody with an invariant or constant region of a human antibody. A humanised antibody is produced by recombinant methods to combine the CDR's of an antibody with both the constant regions and the framework regions from the variable regions of a human antibody.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a further preferred method of the invention said agent is a peptide such as a modified peptide.

In a further preferred method of the invention the peptide has an amino acid sequence encoded by nucleotides 1710 to 2051 of the sequence shown in FIG. 12.

In a further preferred method of the invention the peptide has an amino acid sequence encoded by nucleotides 1356 to 1523 of the sequence shown in FIG. 12.

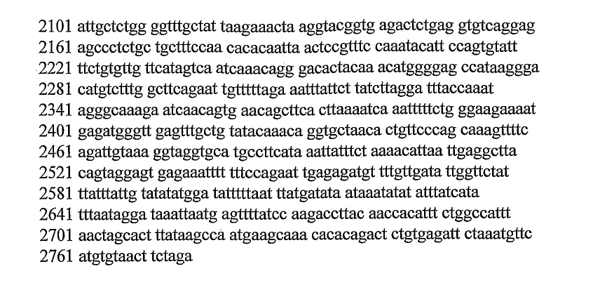

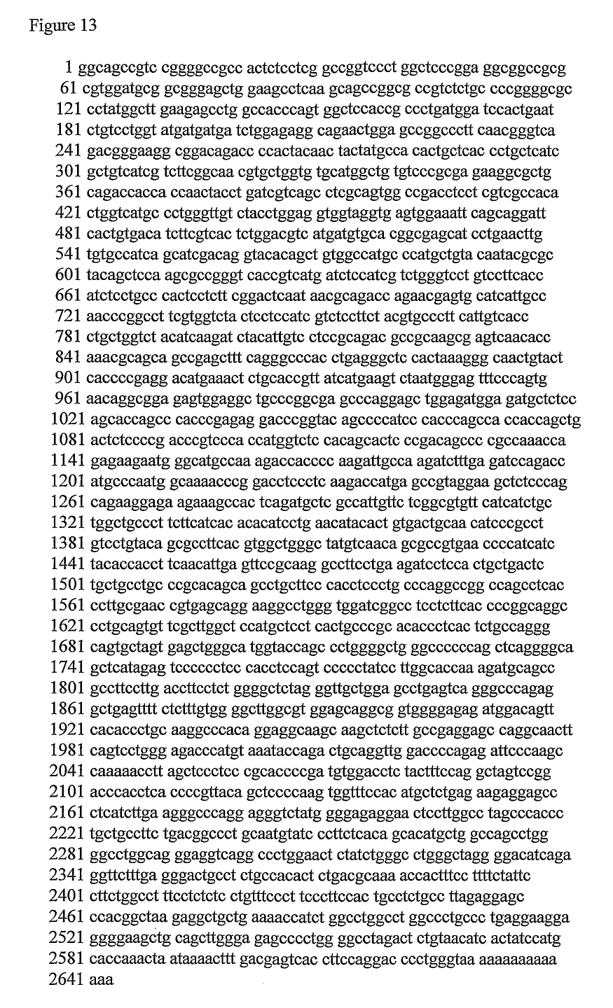

In a further preferred method of the invention the peptide has an amino acid sequence encoded by nucleotides 796 to 1281 of the sequence shown in FIG. 13.

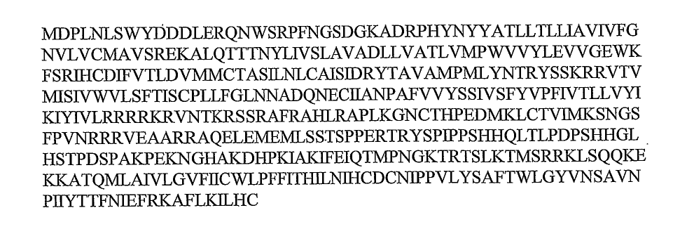

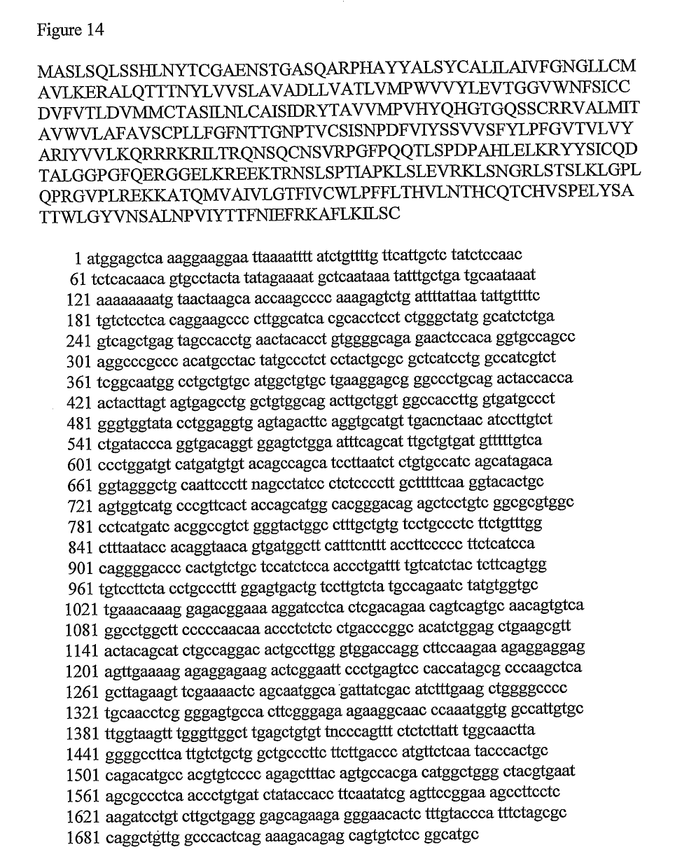

In a further preferred method of the invention the peptide has an amino acid sequence encoded by nucleotides 1005 to 1364 of the sequence shown in FIG. 14.

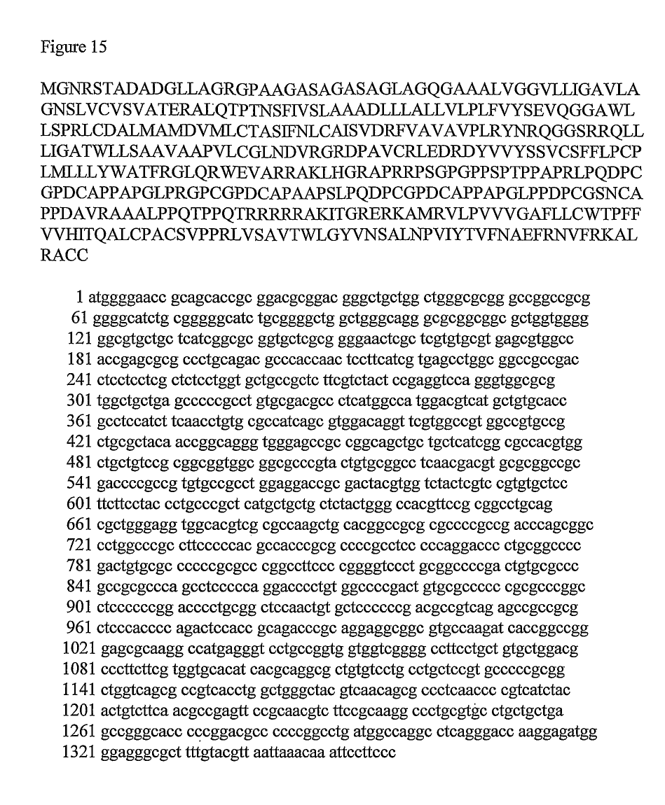

In a further preferred method of the invention the peptide has an amino acid sequence encoded by nucleotides 646 to 1023 of the sequence shown in FIG. 15.

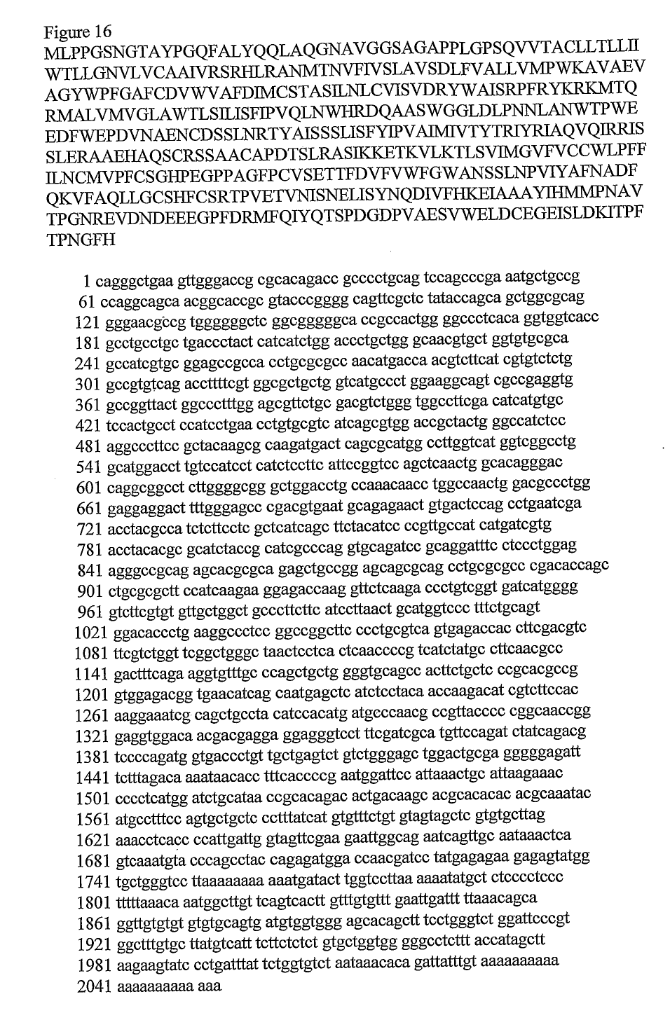

In a further preferred method of the invention the peptide has an amino acid sequence encoded by nucleotides 772 to 948 of the sequence shown in FIG. 16.

It will be apparent to one skilled in the art that modifications to the amino acid sequence of peptides which modulate the interaction of a DRIP with one or more dopamine receptors could enhance the binding and/or stability of the peptide with respect to its target sequence. In addition, modification of the peptide may also increase the in vivo stability of the peptide thereby reducing the effective amount of peptide necessary to inhibit an interaction. This would advantageously reduce undesirable side effects which may result in vivo. Modifications include, by example and not by way of limitation, acetylation and amidation.

In a preferred method of the invention said peptide is acetylated. Preferably said acetylation is to the amino terminus of said peptide.

In a further preferred method of the invention said peptide is amidated. Preferably said amidation is to the carboxyl-terminus of said peptide.

In a further preferred method of the invention said peptide is modified by both acetylation and amidation.

Alternatively, or preferably, said modification includes the use of modified amino acids in the production of recombinant or synthetic forms of peptides. It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂ C₃ or C₄ alkyl R group optionally substituted by 1, 2 or 3 substituents selected from halo (e.g. F, Br, I), hydroxy or C₁-C₄ alkoxy.

Alternatively, peptides could be modified by, for example, cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

In a preferred method of the invention peptides according to the invention are modified by cyclisation.

In a still further alternative embodiment of the invention said agent is an aptamer.

Nucleic acids have both linear sequence structure and a three dimensional structure which in part is determined by the linear sequence and also the environment in which these molecules are located. Conventional therapeutic molecules are small molecules, for example, peptides, polypeptides, or antibodies, which bind target molecules to produce an agonistic or antagonistic effect. It has become apparent that nucleic acid molecules also have potential with respect to providing agents with the requisite binding properties which may have therapeutic utility. These nucleic acid molecules are typically referred to as aptamers. Aptamers are small, usually stabilised, nucleic acid molecules which comprise a binding domain for a target molecule, in the present invention a polypeptide comprising a scavenger receptor cysteine rich domain. A screening method to identify aptamers is described in U.S. Pat. No. 5,270,163 which is incorporated by reference. Aptamers are typically oligonucleotides which may be single stranded oligodeoxynucleotides, oligoribonucleotides, or modified oligodeoxynucleotide or oligoribonucleotides.

The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include by example and not by way of limitation; alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N-6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N-6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine.

In a further embodiment, the agent is an interfering RNA (RNAi) molecule. Preferably said RNAi molecule is derived from the nucleic acid molecule according to the first aspect of the invention. More preferably said RNAi molecule according has a length of between 10 nucleotide bases (nb) −1000 nb. Even more preferably said RNAi molecule has a length of 10 nb; 20 nb; 30 nb; 40 nb; 50 nb; 60 nb; 70 nb; 80 nb; 90 nb; or 100 bp. Even more preferably still said RNAi molecule is 21 nb in length.

The aptamers of the invention may be synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—. The binding of aptamers to a target polypeptide is readily tested by assays hereindisclosed.

The method of the invention enables the identification of agents which modulate (for example inhibit, reduce, block or promote) the interaction between a polypeptide according to the invention and a dopamine receptor. An agent may be as described herein or the agent may be a small molecule. For example, such small molecules include, but are not limited to, peptides, peptidomimetics (e.g., peptoids), amino acids, amino acid analogs, polynucleotides, polynucleotide analogs, nucleotides, nucleotide analogs having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The invention further provides a screening assay for agents which may affect the interactions of a DRIP with downstream factors with which it interacts. For example, the interactions of DRIPs with other proteins involved in dopamine signalling, for example DARPP-32 and CREB as described herein, may be regulated by such agents.

The invention further provides the use of a nucleic acid molecule according to the first aspect of the invention in research. For example, a nucleic acid molecule may be used as a starting point in studies whereby one or more changes are made relative to a given nucleic acid molecule. This can be done to determine which parts thereof, for example the DRIP binding domain, are important in the interaction of DRIP with a dopamine receptor or other binding protein.

According to a further aspect of the invention there is provided a cell transfected with at least one nucleic acid molecule wherein the genome of said cell is modified to include at least one copy of a nucleic acid molecule encoding a polypeptide selected from the group consisting of
i) a polypeptide, or fragment or variant thereof, encoded by a nucleic acid molecule consisting of a nucleic acid sequence as represented by FIG. 1, 2, 3, 4, 5, 6, 7, 8, 9 10 or 11 or a sequence complementary thereto, or a fragment thereof;
ii) a polypeptide encoded by a nucleic acid molecule which hybridises to a nucleic acid molecule as defined in (i) above and which has dopamine receptor binding activity;
iii) a polypeptide comprising a nucleic acid which is degenerate as a result of the genetic code to the nucleic acid sequence defined in (i) and (ii)
wherein said cell is adapted for the regulated expression of said nucleic acid molecule.

Expression of the nucleic acid molecule may be up-regulated or down-regulated. Thus, the cell/cell lines may over-express one or more DRIPs or may be deficient in one or more DRIPs as a result of DRIP knock out.

In a preferred aspect of the invention said cell is further transfected with at least one nucleic acid molecule wherein the genome of said cell is modified to include at least one copy of a nucleic acid molecule encoding one or more dopamine receptors as described herein.

In a preferred embodiment of the invention said cell further comprises a nucleic acid molecule which includes a reporter gene to monitor the activity of said polypeptide(s).

The cells of the invention may be useful in a screening method according to the present invention. Agents may be identified by the screening method using a number of techniques known to the skilled person, for example, GST pull-down assays, coimmunoprecipitation and sub-cellular localisation and co-localisation by immunofluorescence techniques as described in the Examples herein. Surface plasmon resonance technology (BIACORE) may be used to detect molecular interactions on the surface of a chip giving reaction kinetics and quantitative data on binding affinities.

Preferred cells include E. coli, yeast, filamentous fungi, insect cells, mammalian cells, preferably immortalised such BE 93, CHO, HeLa, Myeloma, Jurkat or cos cell lines, monkey cell lines and derivatives thereof.

In a preferred method of the invention said cell is a brain cell, for example, a neuronal cell.

According to a yet further aspect of the invention there is provided a transgenic non-human animal comprising at least one cell according to the invention. Transgenic animals are useful for the analysis of DRIPs and their phenotypic effect. Expression of a nucleic acid sequence as defined in the first aspect of the invention in a transgenic non-human animal is usually achieved by operably linking the polynucleotide to a promoter and/or enhancer sequence and introducing this into an embryonic stem cell of a host animal. The invention includes transgenic non-human animals comprising cells in which one or more nucleic acid sequences as represented by FIG. 1, 2, 3, 4, 5, 6, 7, 8, 9 10 or 11 are over-expressed. Over-expression of the nucleic acid sequence can be achieved by microinjection of the sequence, plus regulatory elements, into the pronucleus of the oocyte (Hogan et al., A Laboratory Manual, Cold Spring harbour and Capecchi Science (1989) 244: 1288-1292). The transgene construct should then undergo homologous recombination with the endogenous gene of the host. Those embryonic stem cells comprising the desired polynucleotide sequence may be selected, usually by monitoring expression of a marker gene, and used to generate a non-human transgenic animal. The invention also encompasses a transgenic animal comprising a cell in which one or more nucleic acid sequences as represented by FIG. 1, 2, 3, 4, 5, 6, 7, 8, 9 10 or 11 is knocked out, for example, by introducing the sequence, plus regulatory elements, containing a selectable marker into embryonic stem (ES) cells by transfection. In such “knock out” animals, expression of the nucleic acid sequence according to the invention is prevented.

In a further preferred embodiment of the invention said transgenic animal is a rodent e.g a rat, mouse or hamster. Alternatively, the transgenic animal may be C. elegans, zebrafish, drosophila or xenopus.

A yet further aspect of the invention provides the use of a dopamine receptor in the identification of agents which modulate the interaction of said receptor with a polypeptide selected from the group consisting of:
i) a polypeptide, or fragment or variant thereof, encoded by a nucleic acid molecule consisting of a nucleic acid sequence as represented by FIG. 1, 2, 3, 4, 5, 6, 7, 8, 9 10 or 11, or a sequence complementary thereto, or a fragment thereof;
ii) a polypeptide encoded by a nucleic acid molecule which hybridises to a nucleic acid molecule as defined in (i) above and which has dopamine receptor binding activity;
iii) a polypeptide comprising a nucleic acid which is degenerate as a result of the genetic code to the nucleic acid sequence defined in (i) and (ii).

In a yet further aspect of the invention there is provided the use of a polypeptide in the identification of agents which modulate the interaction of said polypeptide with a polypeptide involved in dopamine signalling wherein the polypeptide is selected from the group consisting of:
i) a polypeptide, or fragment or variant thereof, encoded by a nucleic acid molecule consisting of a nucleic acid sequence as represented by FIG. 1, 2, 3, 4, 5, 6, 7, 8, 9 10 or 11, or a sequence complementary thereto, or a fragment thereof;
ii) a polypeptide encoded by a nucleic acid molecule which hybridises to a nucleic acid molecule as defined in (i) above and which has dopamine receptor binding activity;
iii) a polypeptide comprising a nucleic acid which is degenerate as a result of the genetic code to the nucleic acid sequence defined in (i) and (ii).

The polypeptide involved in dopamine signalling may include, for example, DARPP-32 and CREB.

A yet further aspect of the invention provides the use of a polypeptide in the identification of agents which modulate the interaction of said polypeptide with a dopamine receptor wherein the polypeptide is selected from the group consisting of:
i) a polypeptide, or fragment or variant thereof, encoded by a nucleic acid molecule consisting of a nucleic acid sequence as represented by FIG. 1, 2, 3, 4, 5, 6, 7, 8, 9 10 or 11, or a sequence complementary thereto, or a fragment thereof;
ii) a polypeptide encoded by a nucleic acid molecule which hybridises to a nucleic acid molecule as defined in (i) above and which has dopamine receptor binding activity;
iii) a polypeptide comprising a nucleic acid which is degenerate as a result of the genetic code to the nucleic acid sequence defined in (i) and (ii).

DRIPs can be used for structure-based design of DRIP inhibitors or of molecules which modulate dopamine receptor function such as though the modulation of DRIP binding to a dopamine receptor. Such “structure based design” is also known as “rational drug design”. The DRIPs can be three-dimensionally analysed by, for example, X-ray crystallography, nuclear magnetic resonance or homology modelling, all of which are well-known methods. The use of DRIP structural information in molecular modelling software systems is also encompassed by the invention. Such computer-assisted modelling and drug design may utilise information such as chemical conformational analysis, electrostatic potential of the molecules, protein folding etc. One particular method of the invention may comprise analysing the three-dimensional structure of DRIPs for likely binding sites of targets, synthesising a new molecule that incorporates a predictive reactive site, and assaying the new molecule as described above.

In a further aspect of the invention there is provided a method of preventing or treating a dopamine-mediated, or dopamine related, disorder in a subject wherein the method comprises modulating the interation of a DRIP with one or more dopamine receptors in the subject.

A dopamine-mediated disorder may include neuropsychiatric disorders, including Parkinson's Disease, Attention Deficit Hyperactivity Disorder (ADHD), depression (bipolar disorder) and schizophrenia and addiction (e.g drug addiction such as alcohol, nicotine or cocaine addiction). Other “dopamine-mediated disorders” may include neurodegenerative disorders (e.g Alzheimer's disease, Tourette Syndrome, Parkinson's disease, Huntington's disease, Amyotrophic Lateral Sclerosis, senile chorea, Sydenham's chorea, autism, head and spinal cord trauma, acute and chromic pain, epilepsy and seizures, dementia, distonia, tremor, autism, cerebral ischemia and neuronal cell death) and disorders linked to apoptosis (particularly neuronal apoptosis). Dopamine related disorders may include cardiovascular, pulmonary or renal conditions.

By modulating is meant inhibiting or promoting the interation of a DRIP with one or more dopamine receptors. DRIP interaction with a dopamine receptor may be modulated in a number of ways. For example, expression of the gene may be inhibited through use of antisense sequences ie, the complementary sequences according to the first aspect of the invention. Such sequences when introduced into a subject by gene therapy will hybridise to the DRIP gene or RNA, and inhibit its translation or transcription. This method may be particularly useful where it is desirable to modulate the function of certain splice variants of DRIP whilst not affecting others.

The introduction of a nucleic acid sequence according to the first aspect of the invention may use gene therapy methods including those known in the art. In general, a nucleic acid sequence will be introduced into the target cells of a subject, usually in the form of a vector and preferably in the form of a pharmaceutically acceptable carrier. Any suitable delivery vehicle may be used, including viral vectors, such as retroviral vector systems which can package a recombinant genome. The retrovirus could then be used to infect and deliver the polynucleotide to the target cells. Other delivery techniques are also widely available, including the use of adenoviral vectors, adeno-associated vectors, lentiviral vectors, pseudotyped retroviral vectors and pox or vaccinia virus vectors. Liposomes may also be used, including commercially available liposome preparations such as Lipofectin®, Lipofectamine®, (GIBCO-BRL, Inc. Gaitherburg, Md.), Superfect® (Qiagen Inc, Hilden, Germany) and Transfectam® (Promega Biotec Inc, Madison Wis.).

Also provided is an agent, such as those defined above, for use as a medicament, for example, in the prevention or treatment of a dopamine-mediated, or dopamine related, disorder in a subject as defined above. The agents include antisense sequences and any other agents which are capable of modulating the interactions of DRIPs. Such agents may also be useful in the treatment of disorders characterised by deficient or defective DRIP.

The invention will now be described, by way of example only, with reference to the following figures in which:

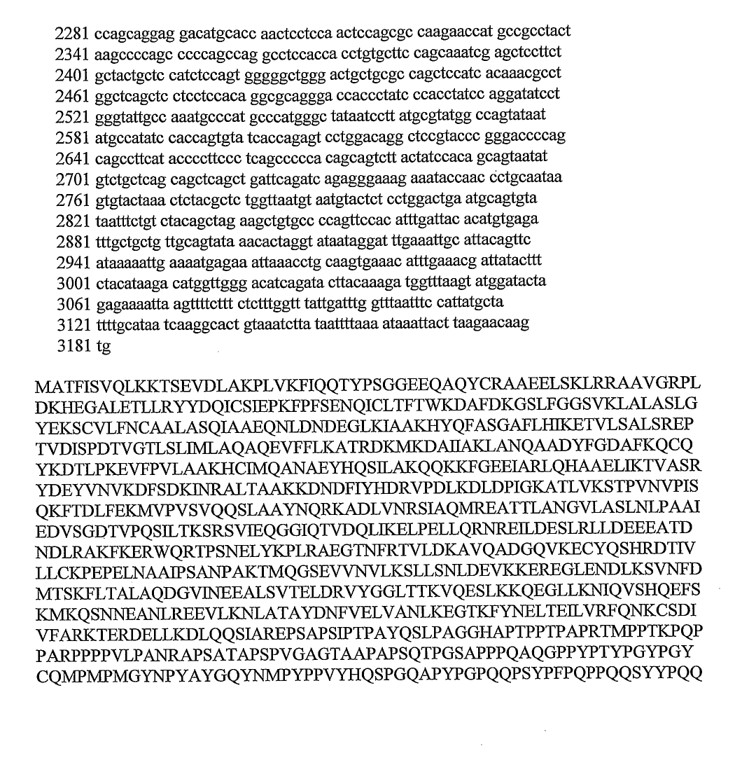

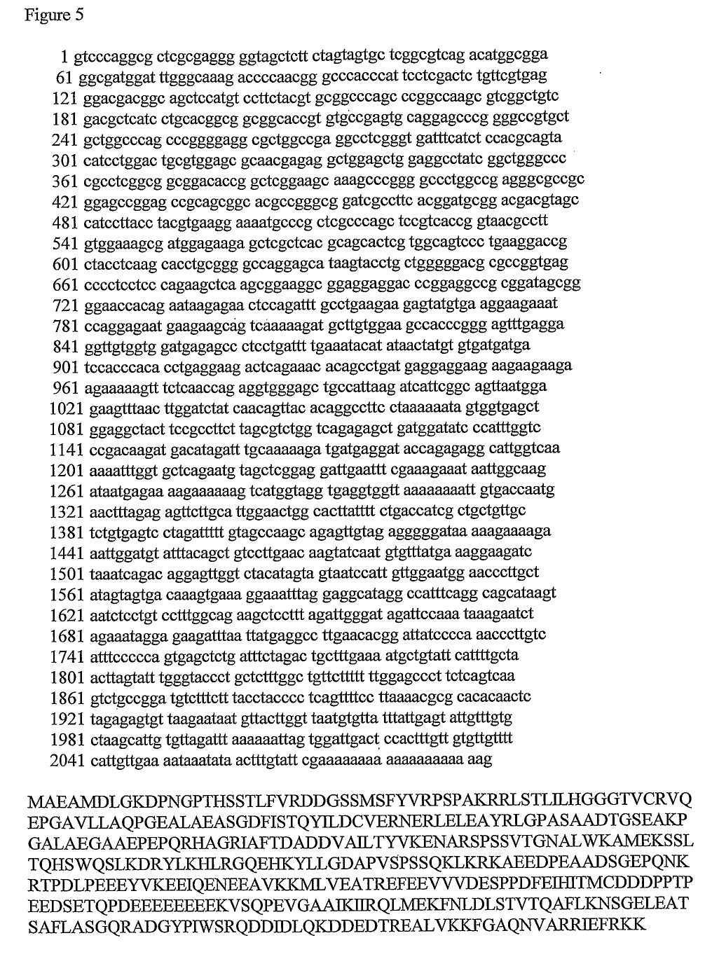

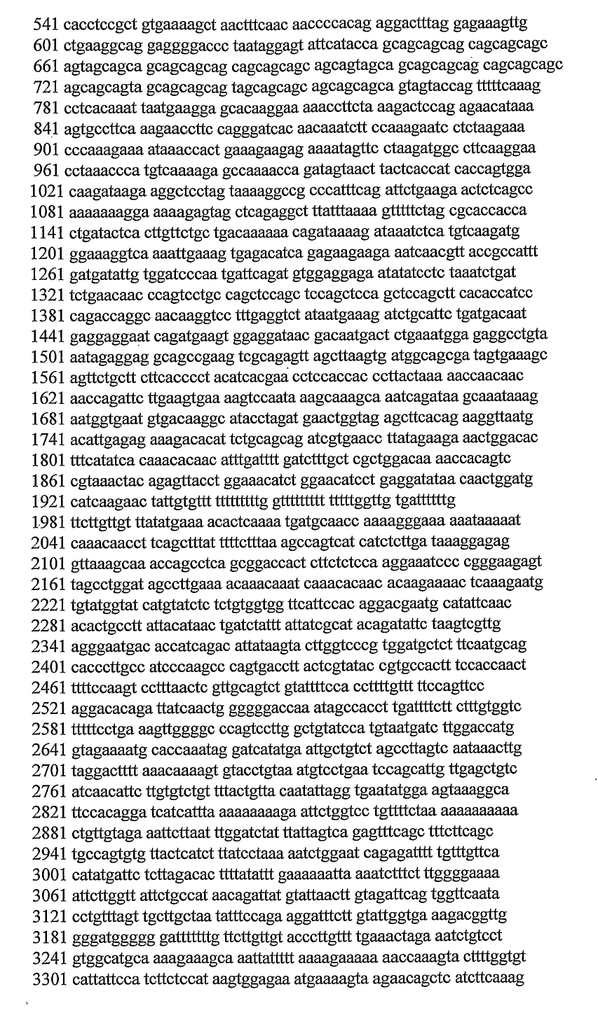

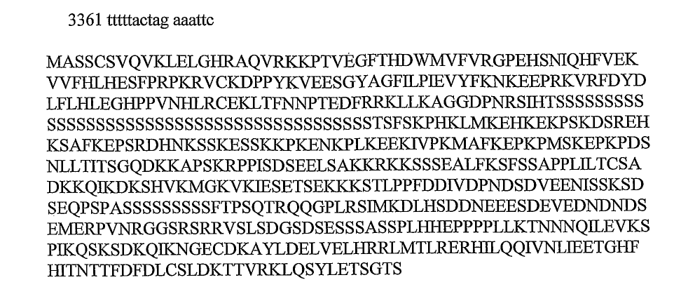

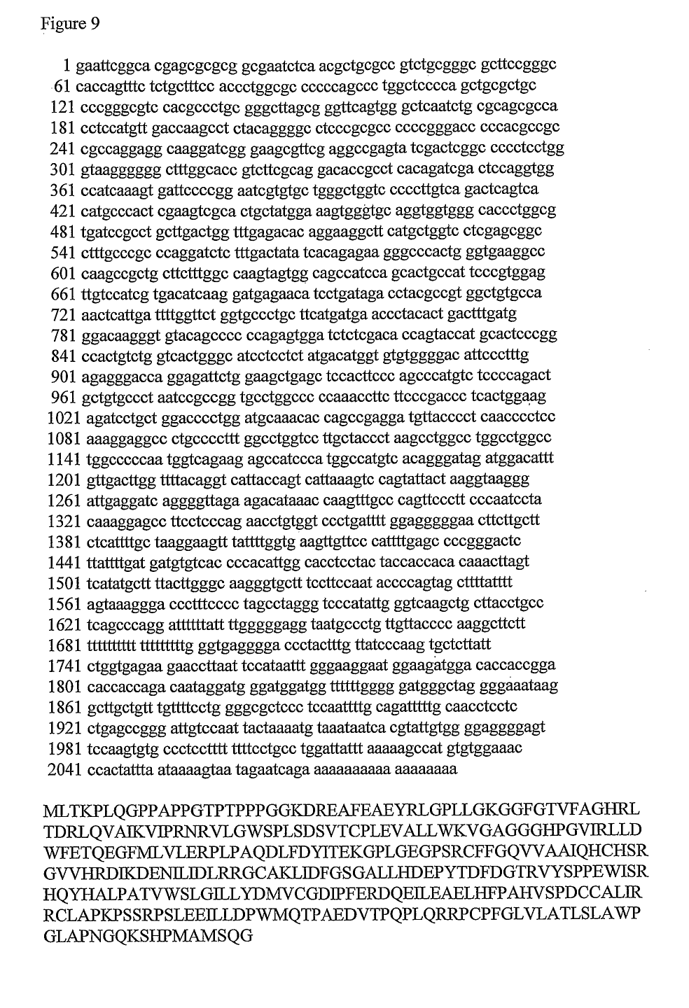

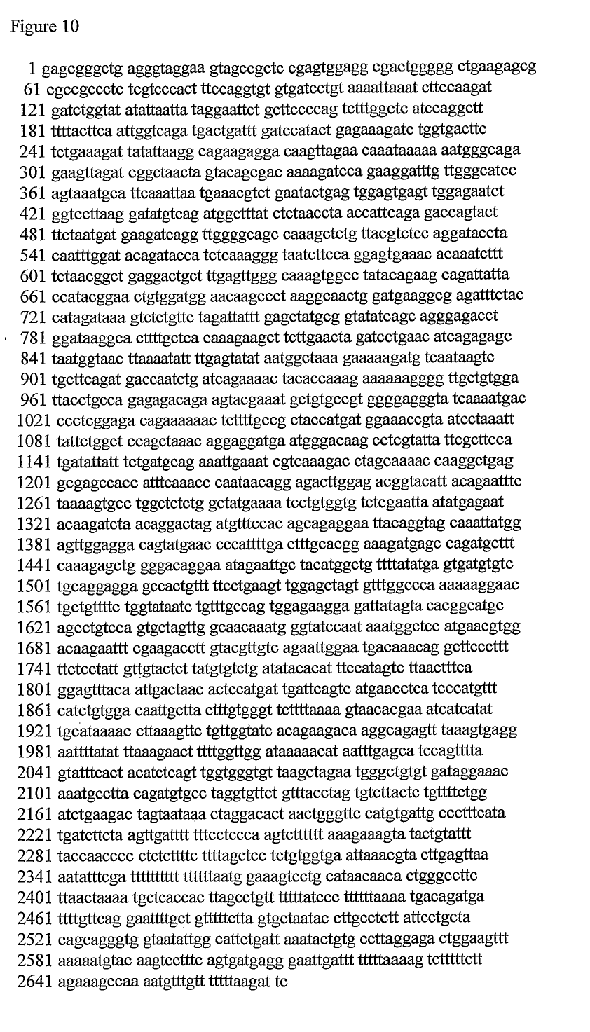

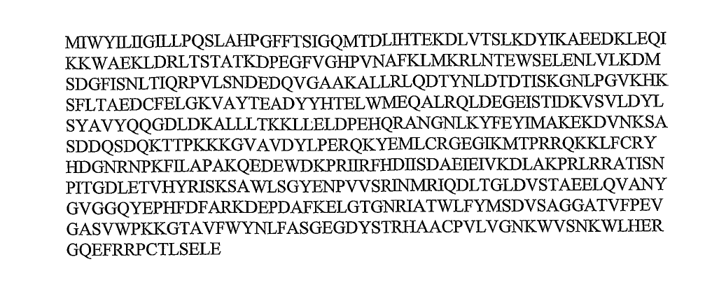

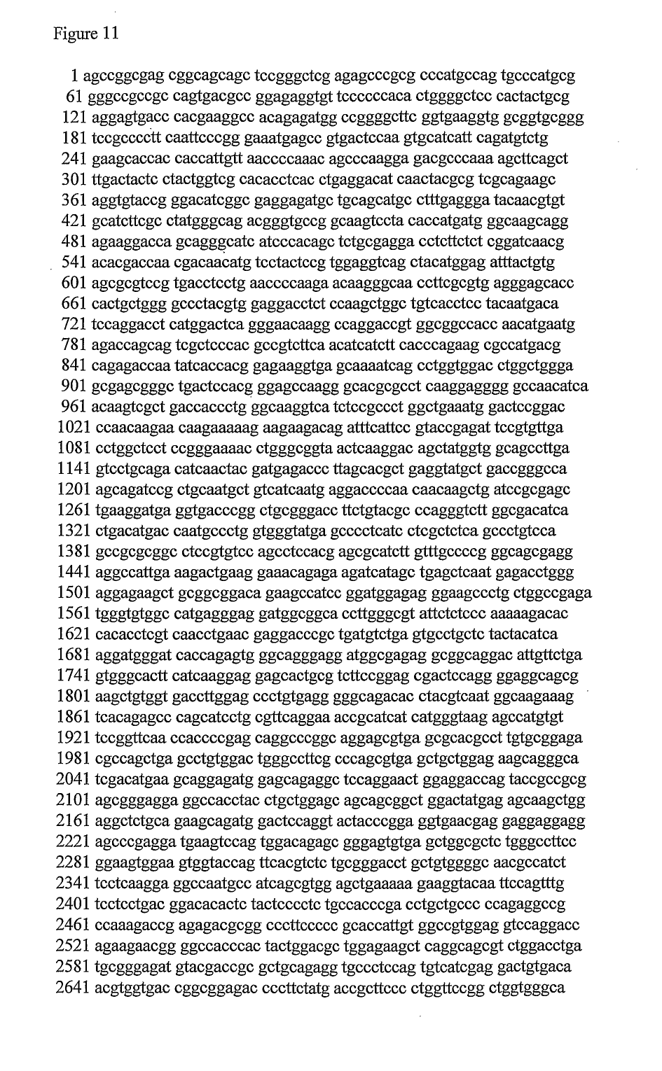

FIGS. 1 to 11 show the DNA (full-length or partial) and amino acid sequences of the dopamine receptor interacting proteins (DRIPs) 1 to 11 respectively. In FIGS. 2, 3, 4 and 8 (A) shows the partial DNA and amino acid sequences and (B) shows the full-length DNA and amino acid sequences;

FIGS. 12 to 16 show the amino acid and DNA sequences of the dopamine receptors D1 to D5 respectively;

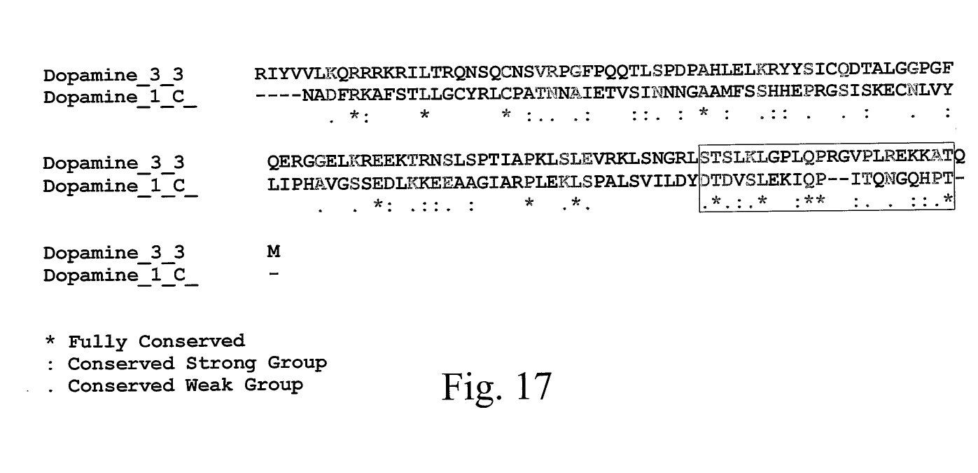

FIG. 17 shows the alignment of D1 C-terminus domain with D3 third cytoplasmic loop. A conserved domain is highlighted.

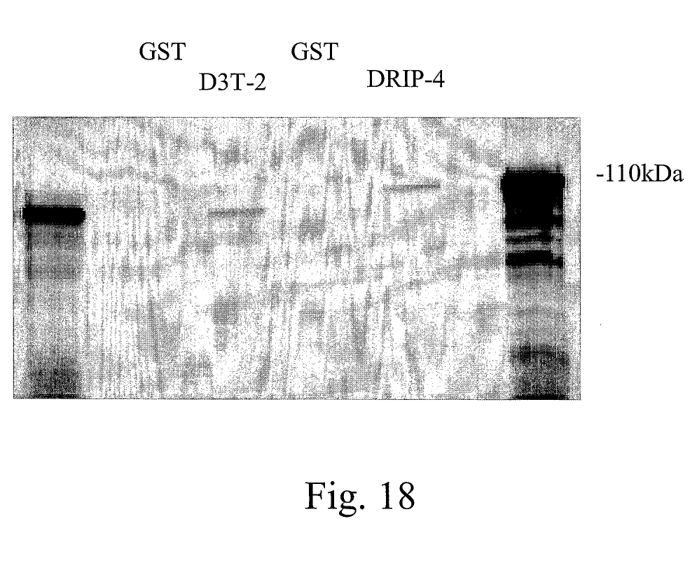

FIG. 18 shows the GST-Pulldown assay. The interaction between both full-length DRIP-4 and its N-terminal truncated version (first 100 amino acids of DRIP4) (D3T-2), with D1 C-terminal GST fusion protein (GST-D1C) is detected by autoradiography. Full-length DRIP-4 and its N-terminal truncated version were transcribed and translated and then incubated with either immobilized GST alone or GST-D1C. Bound proteins were washed and analyzed by SDS-PAGE, followed by autoradiography.

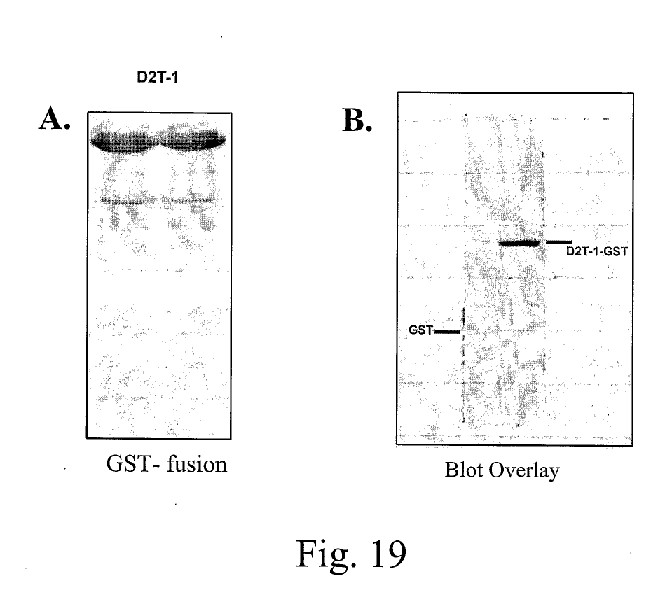

FIG. 19 shows the production and interaction of DRIP-5. a. Comassie-stained acrylamide gel with GST fusion DRIP-5 (D2T-1-GST) protein. B. Blot overlay confirmation of DRIP-5 (D2T-1-GST) versus D2T interaction.

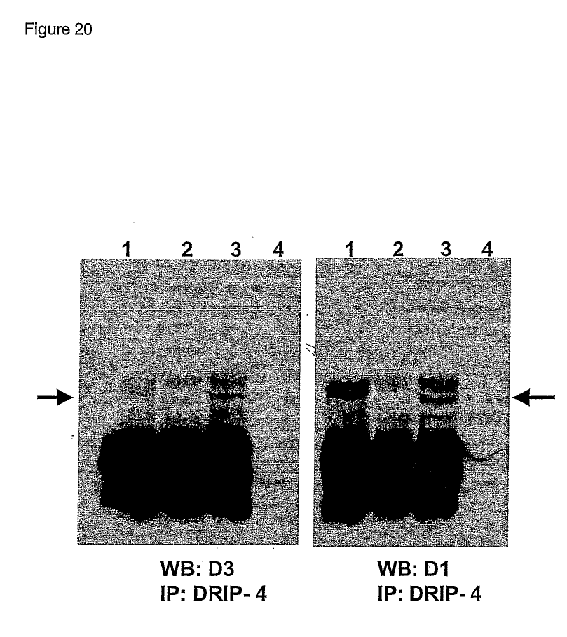

FIG. 20 shows the interaction of DRIP-4 with D1 and D3 in vivo. Lanes 1 and 2 represent immunoprecipitation with irrelevant mouse antibodies, GST and IgG respectively (negative controls). Lane 3=immunoprecipitation with DRIP-4 antibody. Lane 4=mouse brain lysate (positive control). D1 and D3 proteins are indicated by arrows.

### EXAMPLES

### Materials and Methods

### Yeast Two-hybrid Screening

The yeast two-hybrid system was used to assay for in vivo protein-protein interactions (Fields and Song, 1989), using the yeast mating procedure (Bendixen et al., 1994; Fromont-Racine et al., 1997). To identify dopamine receptor interacting proteins (DRIPs), we performed a two-hybrid screening with the dopamine receptor cDNA fused to the GAL4 DNA binding domain (GAL4BD). A plasmid library of fusion between the GAL4 transcription activation domain (GAL4AD) and cDNAs from human brain was screened for interaction with GAL4BD-DR fusion proteins in the yeast reporter strain pJ694a.

The C-terminal end of D1DR was used as a bait in the two-hybrid screening. The coding sequence for the last 114 amino acids of D1DR (Asn234-TGA447) was amplified using PCR employing the following oligonucleotides as primers 5′-CGCGAATTCAATGCTGATTTTCGGAAGG-3′ and 5-CGCCTGCAGTCAGGTTGGGTGCTG-3′. The PCR product was digested with EcoRI and PstI (underlined sequences) and the resulting fragments were subcloned into the yeast two-hybrid expression vector pGBT9 (Bartel et al., 1993) encoding the Gal4 domain to generate D1DR-pGBT9 for yeast two-hybrid screening.

Strain PJ694a, bearing a plasmid expressing a dopamine receptor fusion (DR-pGBT9), was mated to strain pJ694a transformed with a library of fusions between the Gal4 activation domain and diploids with putatively interacting fusion proteins were identified on selective plates lacking histidine, tryptophan and leucine and containing 2 mM 3-aminotriazole. The diploids selected in each set of plates were tested for expression of the other reporter genes. The different pACT2 fusion proteins from the diploids positive for the reporter gene were rescued by complementation of a leuB6 E. coli strain. The fusion genes were sequenced using the oligonucleotide 5′-GGCTTACCCATACGATGTTC-3′ as primer.

We have identified eleven novel Dopamine Receptor Interacting Proteins (DRIP-1 to DRIP-11), from six different baits (D1T, D1C, D2T, D3T, D4T, D5T) in separate screens (Table I). At least one interacting protein was isolated, per ‘bait’. None of the interacting proteins have previously been implicated in dopamine signalling or schizophrenia, but almost all of them map to genomic regions linked to the disease (Table I). For example, DRIP-2 and DRIP-11 map to chromosome 2q, DRIP-7 maps to 5q and DRIP-9 maps to Xp11. Despite the sequence homology between individual receptors, overall each receptor identified a unique set of interacting protein (s). However, D1C and D3T identified the same interacting protein, DRIP-4, indicating that at least D1 and D3 share a common signalling pathway.

TABLE I
Dopamine Receptor Interacting Proteins (DRIPs).

| Interacting | Original | Chromosomal | | |
|---|---|---|---|---|
| Protein | designation | location | Function | Comments |
| DRIP-1 | D1T-4 | 14q21 | unknown | — |
| DRIP-2 | D1T-14 | 2q36 | unknown | homologues in C. elegans. |
| DRIP-3 | D1T-33 | 1p34 | unknown | hypothetical domains |
| | | | | match bacterial sequences. |
| DRIP-4 | D1C-4/D3T-2* | 3p23 | anti-apoptotic | — |
| DRIP-5 | D2T-1 | 16q23 | unknown | homologue in yeast involved |
| | | | | in chromatin assembly and |
| | | | | DNA repair. |
| DRIP-6 | D1T-19 | 11p13 | 2 x LIM domains | — |
| DRIP-7 | D1C-6 | 5q35/21 | G-protein | WD-repeat domain. |
| | | | (βsubunit) | |
| DRIP-8 | D2T-2 | 9p21 | developmental | homeotic gene? |
| DRIP-9 | D5T-3 | Xp11 | kinase domain | active site for serine- |
| | | | | threonine protein kinases. |
| DRIP-10 | D4T-5 | 10q22 | enzyme | TPR motif; WD-40 repeat |
| | | | | Domain. |
| DRIP-11 | D4T-12 | 2q37 | unknown | Expressed in foetal brain, |
| | | | | 6-week embryos, hippocampus + |
| | | | | cerebellum. |

The original designation refers to the original ‘bait’ used in the yeast two-hybrid screen and the clone number. Thus, D3T-2, refers to the third loop (T) of dopamine receptor 3 (D3). D3T-2 was the second clone recovered from the screen.
*D1C-4 and D3T-2 were identified in separate screens with D1C and D3T.

TABLE II
Dopamine Receptor Interacting Proteins (DRIPs).

| ‘BAIT’ | ‘PREY’ | GENE | FUNCTION |
|---|---|---|---|
| D1T-4 | DRIP-1 | c14orf28 | unknown |
| D1T-14 | DRIP-2 | DOCK-10 | signalling (activates GTPases) |
| D1T-33 | DRIP-3 | YRDC | translation |
| D1C-4/D3T-2 | DRIP-4 | AIP-1 | inhibits programmed cell death |
| D2T-1 | DRIP-5 | TERF2IP | telomeric repeat maintenance |
| D1T-19 | DRIP-6 | LMO2 | neuronal development (LIM domains) |
| D1C-6 | DRIP-7 | GNB2L1 | G-protein coupled signalling |
| D2T-2 | DRIP-8 | MLLT3 | development (homeotic gene; trithorax |
| | | | homologue) |
| D5T-3 | DRIP-9 | pim2 | kinase (reverses BAD induced apoptosis; |
| | | | upregulated in brain after trauma) |
| D4T-5 | DRIP-10 | P4HA1 | enzyme (collagen synthesis) |
| D4T-12 | DRIP-11 | KIF1A | molecular motor (involved in |
| | | | neurotransmitter transport) |

AIP-1 was identified in two separate screens using D1C and D3T as a ‘bait’ (Miki et al., (2003) Genome Res 13, 1455-1465).

The Table below shows which DRIPs correspond to which Dopamine Receptor:

| | |
|---|---|
| Bait | DRIP |

| | | | | |
|---|---|---|---|---|
| D1C | | *DRIP-4, | | DRIP-7 |
| D1T | DRIP-1, | DRIP-2, | DRIP-3, | DRIP-6 |
| D2T | | DRIP-5, | | DRIP-8 |
| D3T | | | | *DRIP-4 |
| D4T | | DRIP-10, | | DRIP-11 |
| D5T | | | | DRIP-9 |

*DRIP-4 was independently identified in separate library screens using D1C and D3T as baits.

### Dopamine Receptor Interacting Proteins (DRIP-1 to DRIP-11).

DRIP-4 interacts with the C-terminal of D1 receptor (D1C) and the third cytoplasmic loop of D3 receptor (D3T), raising the possibility of a common signalling pathway involving D1 and D3. We have evidence confirming the interaction of DRIP-4 with D1 and D3, by coimmunoprecipitation from mouse brain lysates (FIG. 20) and fron HEK293 cells (data not shown). We performed an alignment of D1C with D3T and identified significant homology between these sequences (FIG. 17).

We have obtained experimental evidence confirming the role of DRIP-4 in inhibiting apoptosis and paraptosis, by over-expressing this protein in mammalian cells.

We have shown, using semi-quantitative RT-PCR, that the onset of DRIP-4 expression coincides with the development of the nervous system in mice (data not shown). Therefore, we believe that DRIP-4 could be involved in degeneration of neuronal circuits through failure to suppress neuronal cell death during development.

### Cloning

The protein-protein interactions are being confirmed/tested in mammalian cells, fixed tissue slices and fresh tissue using in vitro and in vivo methods, as described below.

Full-length cDNA constructs have been generated for dopamine receptors and interacting proteins (DRIPs) in a range of expression vectors including, pGEX (GST), pCMVTag (FLAG), pcDNA3.1-Myc-His, pET32 (His) and pEGFP (GFP/YFP/BFP/CFP) series vectors.

### Expression

We have expressed dopamine receptors and interacting proteins as GST fusion proteins in E. coli. Following IPTG induction, we were able to achieve high expression of all GST fusion proteins and we have tested a wide range of conditions to optimise their solubility (data not shown).

### GST Pull-down Assays

This is an in vitro assay for protein-protein interaction, in which one protein is translated with ³⁵S labelled methionine and incubated with its interacting protein that is expressed as a GST fusion protein attached to glutathione sepharose beads. After binding, the labelled protein is eluted from the beads, run on an acrylamide gel, and detected by autoradiography. This has already been done for interactions involving DRIP-4 (FIG. 18) (and for DRIP-5 and DRIP-6 [data not shown]). In addition, the interactions between DRIP-5 and D2 (FIG. 19), and between DRIP-6 and D1T (data not shown) were confirmed by blot overlay experiments.

### Communoprecipitation

Interacting proteins and corresponding dopamine receptors with FLAG or Myc tags, are being co-expressed in IHEK293 cells. Immunoprecipitation with Myc antibody, followed by a Western blot with FLAG antibody and repeating the experiment using FLAG antibody to immunoprecipitate and Myc antibody for Western blotting, has allowed us to characterise some of the interactions. Antibodies against Myc, FLAG and all five dopamine receptors are commercially available. For approximately half the interacting proteins, including DRIP-4, antibodies are also commercially available. We are testing their interactions in vivo using rat brain lysates. DRIP-4 expressed in BEK-293 cells can be detected using antibodies against Xpress and FLAG tags (data not shown).

### Sub-cellular Localisation and Co-localisation by Immunofluorescence

Co-localisation of interacting proteins within the same sub-cellular compartment provides important evidence for their interaction. Each interacting protein and its corresponding dopamine receptor is being co-expressed in HEK293 cells and cells of neuronal origin such as PC12. The individual proteins can be detected using fluorescent secondary antibodies (coupled to Texas Red or FITC dyes) directed against Myc, FLAG or other tags. The proteins can be co-localised by merging the images from FITC and Texas Red signals under fluorescence or confocal microscopy. This has already been done for D1/D3 versus DRIP-4 interaction in HEK293 and rat primary neuronal cultures (data not shown). In parallel, where suitable antibodies are available (see above), similar experiments are being performed on frozen and paraffin embedded rat brain sections by immunohistochemistry. Furthermore, immunohistochemical analysis on human brain autopsy material and tissue are being conducted.

### Expression Analysis

The expression patterns of the dopamine receptor interacting proteins are being analysed by Northern blotting; quantitative RT-PCR (with mRNA derived from different mouse tissues and different stages of murine embryogenesis); western blotting and immunohistochemistry.

Investigating the role of DRIPs in Dopamine Signalling

G-protein coupled receptors transduce extracellular signals through activation of second messengers. We have measured intracellular cAMP levels in BEK2932 cell lines expressing D1 and in cell lines expressing D3 (data not shown). We will study ligand binding in the presence or absence of specific dopamine receptor agonists and antagonists, using HEK-293 stable cell lines that we have developed that over-express DRIPs or dopamine receptors. We are also using these cell lines and cultured neurons to record whole-cell currents. We have generated stable cell lines over-expressing D1 by 2-3 fold and DRIP-4 by approximately 100 fold (data not shown).

### Investigating the Role of DRIPs in Schizophrenia

We are over expressing DRIP-6, DRIP-8 and DRIP-9 in neuronal cell lines. These cell culture models will be used to study neurite growth and extension. We are planning further experiments to be performed in stem cells to see if over-expressing DRIPs can alter the programme of neural development, because this could have an important impact on brain development and schizophrenia. We are also aiming to assess the potential of specific proteins such as DRIP-4 to support, promote, activate or trigger neuronal growth and differentiation or cell death in stem cells.

Genetic Study to Test for Association of Polymorphisms in DRIPs with Schizophrenia

An association study with dopamine receptor interacting proteins is being carried out, using a well characterised collection of DNA samples of Scottish origin, consisting of 500 schizophrenic cases and 500 controls plus 80 parent proband trios that will assist in determining haplotypes. Positive associations will be tested in forty 2-3 generation families to look for segregation of illness with specific haplotypes.

### Animal Models for Use in Investigating the Role of DRIPs in Dopamine Signalling

An animal model can provide valuable insights into the pathophysiology of a disease. In particular, mouse models are generally sufficiently genetically and physiologically similar to humans to enable the manipulation of genes in vivo. To ascertain gene function, both over-expression or complete inactivation of a DRIP (knock-out) as described herein can be suitable strategies. Over-expression is achieved by microinjecting a gene construct (consisting of the gene of interest and a suitable promoter) into the male pronucleus of a single-cell stage embryo. Gene knock-outs involve a two-stage process. In the first stage, a genomic fragment containing the gene (typically disrupted with a drug resistane marker such as Neo) is constructed. This is transfected into pluripotent embryonic stem (ES) cells. Cells which have incorporated the construct can be selected by adding the drug G418 to select for the Neo marker which offers resistance to the drug allowing the cells to survive in the presence of toxic doses of the drug. The natural process of homologous recombination enables the Neo cassette to replace the endogenous gene in some cells. These cells are selected using a PCR strategy. In the second stage, ‘positive’ cells, i.e. those cells in which the endogenous wild-type gene has been replaced by the Neo cassette containing the disrupted (mutant) copy of the gene, are injected into blastocyst stage donor embryos which are subsequently introduced into pseudopregnant females. The resulting animals are chimeras consisting of wild-type and mutant cells. By a process of breeding, pure mutant animals that are viable can be generated.

### REFERENCES

Abi-Dargham, A., Rodenhiser, J., Printz, D., Zea-Ponce, Y., Gil, R., Kegeles, L. S., Weiss, R. et al (2000). Proc. Natl. Acad. Sci. USA. 97, 8104-8109.
Baik, J-H, Picetti, R., Saiardi, A., Thiriet, G., Dierich, A., Depaulis, A., Le Meur, M. and Borrelli, E. (1995) Nature 377, 424-428.
Bartel P. L., Chien C. t., Stemglanz R. and Fields S. (1993). In Cellular interaction in development: A practical approach., D. A. Hartley, ed. (Oxford Unversity Press, Oxford), pp 153-179
Bendixen C., Gangloff S, and Rothstein R. (1994). Nucleic Acids Res 22:1778-1779
Carlsson, A. (2001 Science 294, 1021-1024.
Coon, H., Byerley, W., Holik, J., Hoff, M. et al. (1993) Am. J. Hum. Genet. 52, 327-334.
Fields, S. and Song, O. (1989) Nature 340, 245-247.
Fromont-Racine M., Rain J. C. and Legrain P. Nat genet (1997) 16:277-282
Giros, B., Jaber, M., Jones, S. R., Wightman, R. M. and Caron, M. G. (1996) Nature 379, 606-612.
Greengard, P. (2001) The neurobiology of slow synaptic transmission. Science 294, 1024-1030.
Kabbani, N., Jeromin, A. and Levenson, R. (2004) Cellular signalling. 16, 497-503.
Lamey, M., Thompson, M., Varghese, G., Chi, H., Sawzdargo, M., George, S. R. and O'Dowd, B. F. (2002) J. Biol. Chem. 277, 9415-9421.
Missale, C., Nash, S. R., Robinson, S. W., Jaber, M. and Caron, M. G. (1998) Physiological Reviews 78, 189-225.
Neves, S. R., Ram, P. T. and Iyengar, R. (2002) Science 296, 1636-1639.
Okubo, Y., Suhara, T., Suzuki, K. et al. (1997) Decreased prefrontal D1 receptors in schizophrenia revealed by PET. Nature 385, 634-636.
Roberts, G. W., Leigh, P. N., and Weinbereger, D. R. (1993) Neuropsychiatric disorders. Wolfe Publishing.
Seeman, P. and Kapur, S. (2000) Proc. Natl. Acad. Sci. USA. 97, 7673-7675.
Seeman, P., Guan, H-C. and Van Tol, H. M. (1993) Nature 365, 441-445.
Sibley, D. R. and Monsana, Jr., F. J. (1992) Trends Pharmacol Sci, 13, 61-69.
Silbersweig, D. A., Stem, E., Frith, C. et al (1995) Nature 378, 176-179.
Wu, G., Benovic, J. L., Hildebrandt, J. D. and Lanier, S. M. (1998) J. Biol. Chem. 273, 7197-7200.

1. A screening method for the identification of agents that modulate the interaction of a polypeptide with one or more dopamine receptors wherein said polypeptide is selected from the group consisting of: i) a polypeptide, or fragment or variant thereof, encoded by a nucleic acid molecule consisting of a nucleic acid sequence as represented by FIG. 1, 2, 3, 4, 5, 6, 7, 8, 9 10 or 11, or a sequence complementary thereto, or a fragment thereof; ii) a polypeptide encoded by a nucleic acid molecule which hybridises to a nucleic acid molecule as defined in (i) above and which has dopamine receptor binding activity; and iii) a polypeptide comprising a nucleic acid which is degenerate as a result of the genetic code to the nucleic acid sequence defined in (i) and (ii); comprising the steps of i) forming a preparation comprising said polypeptide and a dopamine receptor; and ii) adding at least one candidate agent to be tested; iii) determining the effect, or not, or said agent on the interaction of said polypeptide with said dopamine receptor.

2. A method as claimed in claim 1 wherein the polypeptide is represented by the amino acid sequence as shown in FIG. 1, 2, 3, 4, 5, 6, 7, 8, 9 10 or 11, or a variant thereof.

3. A method as claimed in claim 1 wherein the nucleic acid molecule anneals under stringent hybridisation conditions to the nucleic acid sequence shown in FIG. 1, 2, 3, 4, 5, 6, 7, 8, 9, 10 or 11 or to its complementary strand.

4. A method as claimed in claim 1 wherein the nucleic acid sequence comprises nucleotides 644 to 1191 of the sequence shown in FIG. 1.

5. A method as claimed in claim 1 wherein the nucleic acid sequence comprises nucleotides 1 to 601 of the sequence shown in FIG. 2.

6. A method as claimed in claim 1 wherein the nucleic acid sequence comprises nucleotides 1 to 521 of the sequence shown in FIG. 3.

7. A method as claimed in claim 1 wherein the nucleic acid sequence comprises nucleotides 415 to 910 of the sequence shown in FIG. 4.

8. A method as claimed in claim 1 wherein the nucleic acid sequence comprises nucleotides 1058 to 1661 of the sequence shown in FIG. 6.

9. A method as claimed in claim 1 wherein the nucleic acid sequence comprises nucleotides 621 to 1069 of the sequence shown in FIG. 7.

10. A method as claimed in claim 1 wherein the dopamine receptor is represented by a polypeptide having the amino acid sequence shown in FIG. 12, 13, 14, 15 or 16.

11. An agent wherein said agent modulates the interaction of a polypeptide with said dopamine receptor wherein said polypeptide is selected from the group consisting of: i) a polypeptide, or fragment or variant thereof, encoded by a nucleic acid molecule consisting of a nucleic acid sequence as represented by FIG. 1, 2, 3, 4, 5, 6, 7, 8, 9 10 or 11, or a sequence complementary thereto, or a fragment thereof; ii) a polypeptide encoded by a nucleic acid molecule which hybridises to a nucleic acid molecule as defined in (i) above and which has dopamine receptor binding activity; and iii) a polypeptide comprising a nucleic acid which is degenerate as a result of the genetic code to the nucleic acid sequence defined in (i) and (ii).

12. An agent as claimed in claim 11 wherein the agent binds a dopamine receptor and wherein the agent is not the natural ligand of a dopamine receptor.

13. An agent as claimed in claim 11 wherein said agent is an antibody or an active binding fragment of a monoclonal antibody.

14. An agent as claimed in claim 13 wherein the antibody fragment is a single chain antibody variable region fragment.

15. An agent as claimed in claim 13 wherein the antibody or fragment binds a polypeptide comprising an amino acid sequence as represented by FIG. 1, 2, 3, 4, 5, 6, 7, 8, 9 10 or 11, or a variant thereof.

16. An agent as claimed in claim 13 wherein the antibody fragment binds a polypeptide encoded by nucleotides 644 to 1191 of the sequence shown in FIG. 1.

17. An agent as claimed in claim 13 wherein the antibody fragment binds a polypeptide encoded by nucleotides 1 to 601 of the sequence shown in FIG. 2.

18. An agent as claimed in claim 13 wherein the antibody fragment binds a polypeptide encoded by nucleotides 1 to 521 of the sequence shown in FIG. 3.

19. An agent as claimed in claim 13 wherein the antibody fragment binds a polypeptide encoded by nucleotides 415 to 910 of the sequence shown in FIG. 4.

20. An agent as claimed in claim 13 wherein the antibody fragment binds a polypeptide encoded by nucleotides 1058 to 1661 of the sequence shown in FIG. 6.

21. An agent as claimed in claim 13 wherein the antibody fragment binds a polypeptide encoded by nucleotides 621 to 1069 of the sequence shown in FIG. 7.

22. An agent as claimed in claim 13 wherein the antibody fragment binds a polypeptide encoded by nucleotides 1710 to 2051 of the sequence shown in FIG. 12.

23. An agent as claimed in claim 13 wherein the antibody fragment binds a polypeptide encoded by nucleotides 1356 to 1523 of the sequence shown in FIG. 12.

24. An agent as claimed in claim 13 wherein the antibody fragment binds a polypeptide encoded by nucleotides 796 to 1281 of the sequence shown in FIG. 13.

25. An agent as claimed in claim 13 wherein the antibody fragment binds a polypeptide encoded by nucleotides 1005 to 1364 of the sequence shown in FIG. 14.

26. An agent as claimed in claim 13 wherein the antibody fragment binds a polypeptide encoded by nucleotides 646 to 1023 of the sequence shown in FIG. 15.

27. An agent as claimed in claim 13 wherein the antibody fragment binds a polypeptide encoded by nucleotides 772 to 948 of the sequence shown in FIG. 16.

28. An agent as claimed in claim 13 wherein the antibody fragment is a chimeric antibody.

29. An agent as claimed in claim 13 wherein the antibody fragment is a humanised antibody.

30. An agent as claimed in claim 11 wherein the agent is a peptide such as a modified peptide.

31. An agent as claimed in claim 30 wherein the peptide has an amino acid sequence encoded by nucleotides 1710 to 2051 of the sequence shown in FIG. 12.

32. An agent as claimed in claim 30 wherein the peptide has an amino acid sequence encoded by nucleotides 1356 to 1523 of the sequence shown in FIG.

33. An agent as claimed in claim 30 wherein the peptide has an amino acid sequence encoded by nucleotides 796 to 1281 of the sequence shown in FIG.

34. An agent as claimed in claim 30 wherein the peptide has an amino acid sequence encoded by nucleotides 1005 to 1364 of the sequence shown in FIG.

35. An agent as claimed in claim 30 wherein the peptide has an amino acid sequence encoded by nucleotides 646 to 1023 of the sequence shown in FIG.

36. An agent as claimed in claim 30 wherein the peptide has an amino acid sequence encoded by nucleotides 772 to 948 of the sequence shown in FIG. 16.

37. An agent as claimed in claim 11 wherein the agent is an aptamer.

38. An agent as claimed in claim 11 wherein the agent is an interfering RNA (RNAi) molecule.

39. A cell transfected with at least one nucleic acid molecule wherein the genome of said cell is modified to include at least one copy of a nucleic acid molecule encoding a polypeptide selected from the group consisting of i) a polypeptide, or fragment or variant thereof, encoded by a nucleic acid molecule consisting of a nucleic acid sequence as represented by FIG. 1, 2, 3, 4, 5, 6, 7, 8, 9 10 or 11 or a sequence complementary thereto, or a fragment thereof; ii) a polypeptide encoded by a nucleic acid molecule which hybridises to a nucleic acid molecule as defined in (i) above and which has dopamine receptor binding activity; iii) a polypeptide comprising a nucleic acid which is degenerate as a result of the genetic code to the nucleic acid sequence defined in (i) and (ii) wherein said cell is adapted for the regulated expression of said nucleic acid molecule.

40. A cell as claimed in claim 39 wherein said cell is further transfected with at least one nucleic acid molecule wherein the genome of said cell is modified to include at least one copy of a nucleic acid molecule encoding one or more dopamine receptors.

41. A cell as claimed in claim 39 wherein said cell is a brain cell.

42. A cell as claimed in claim 39 wherein said cell is a neuronal cell.

43. A transgenic non-human animal comprising at least one cell as claimed in claim 39.

44. (canceled)

45. (canceled)

46. (canceled)

47. (canceled)

48. (canceled)

49. (canceled)

50. (canceled)

51. (canceled)

52. (canceled)

53. (canceled)
